(19) United States
(12) Patent Application Publication
(10) Pub. No.: US 2026/0215420 A1
(43) Pub. Date: Jul. 30, 2026

(54) INSECT NEUROPEPTIDE ANALOGUES

(71) Applicant: Solasta Bio Limited, Glasgow Central Scotland (GB)

(72) Inventors: Julian A.T. Dow, East Dunbartonshire Central Scotland (GB); Shireen A. Davies, East Dunbartonshire Central Scotland (GB); Yousef Abul-Haija, East Dunbartonshire Central Scotland (GB); Lewis Archibald, Glasgow Central Scotland (GB)

(73) Assignee: Solasta Bio Limited, Glasgow Central Scotland (GB)

(21) Appl. No.: 19/121,653

(22) PCT Filed: Oct. 23, 2023
(86) PCT No.: PCT/GB2023/052767

§ 371 Date: Apr. 16, 2025

(30) Foreign Application Priority Data
Oct. 25, 2022 (GB) 2215797.8

(51) Int. Cl. A01N 37/46; A01N 25/02; A01P 7/04; C07K 7/06; C07K 7/08

(52) U.S. Cl. CPC A01N 37/46; A01N 25/02; A01P 7/04; C07K 7/06; C07K 7/08

(57) ABSTRACT

The present invention relates to analogues of insect neuropeptides having activity against insects, such as insects of the order diptera, hemiptera, blattodea and/or lepidoptera, and their use as insect control agents (e.g. insecticides) and plant protection agents.

### CROSS-REFERENCE TO RELATED APPLICATIONS

This application is a National Phase of International Application No. PCT/GB2023/052767, filed Oct. 23, 2023, which designated the U.S. and that International Application was published under PCT Article 21(2) in English. This application also includes a claim of priority under 35 U.S.C. § 119(a) and § 365(b) to British patent application No. 2215797.8 filed Oct. 25, 2022, the entirety of which is hereby incorporated by reference.

### FIELD OF THE INVENTION

The present invention relates to analogues of insect neuropeptides having activity against insects, such as diptera, hemiptera, blattodea and/or lepidoptera insects, and their use as insect control agents (e.g. insecticides) and plant protection agents.

### BACKGROUND

With a global dependence on broad-spectrum insecticides, the damaging effects of which are well documented, there is increasing need for the development of greener, more selective insecticides. The development and employment of neuropeptide synthetic analogues offers a promising avenue in the drive for greener and more selective insecticidal agents.

Within the insects, neuropeptides are regulatory peptides with functional roles in growth and development, behaviour and reproduction, metabolism and homeostasis, and muscle movement.

Due to their high specificity, neuropeptides and their cognate receptors (typically G-protein coupled receptors, GPCRs) may be developed towards insecticidal agents to selectively reduce the fitness of target pest insects, whilst minimising detrimental environmental impacts, and minimising harm to other non-target species such as pollinators, which have seen dangerously declining population numbers due to pesticide/insecticide use.

Insect neuropeptide families include the insect kinins and cardio acceleratory peptides (CAPA, CAP2b) neuropeptides.

The present invention provides kinin peptides and analogues.

### SUMMARY OF THE INVENTION

The inventors have discovered new analogues of kinin peptides having insecticidal activity against insects that encode kinin peptides, such as insects of the order diptera, hemiptera, blattodea and/or lepidoptera, and so potentially finding use as pest control agents or insecticides, while having little or no effect against important pollinator species such as bees. The inventors have also discovered new kinin peptides.

In a first aspect, the invention provides an insecticidal compound having the formula (XX) below:

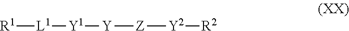

wherein
R¹ is hydrogen (which may be designated “H-” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}, —C(═N⁺(R^{1b})(R^{1c}))NR^{1d}R^{1e} acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀ aryl, biotin, a sugar moiety, (poly)alkyleneglycol, heterocyclyl, phosphate or sulphate;
wherein each of R^{1a}, R^{1b}, R^{1c}, R^{1d} and Rie is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl, a sugar moiety, phosphate or sulphate;
L¹ is absent or is:
   *—(C═O)C₁₋₁₆-alkylene-NH— where * denotes the point of attachment to Y¹, Y or Z;
   (C₁₋₂₀)alkylene,
   (C₂₋₂₀)alkenylene,
   (poly)alkyleneglycol;
wherein L¹ is optionally substituted with one or more groups selected from oxo (═O), halogen, ═N and ═S;
Y¹ is absent or is a peptide comprising from 1 to 2 amino acids;
Y is absent or is a peptide comprising from 1 to 16 amino acids; wherein Y optionally comprises a (poly)alkyleneglycol linker in the peptide sequence;
Y² is absent or is a peptide comprising from 1 to 2 amino acids;
Z is a peptide of the formula:

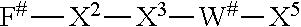

wherein:
   X² is selected from S^{#}, Y^{#}, K^{#}, N^{#} or H^{#};
   X³ is selected from S^{#}, A^{#}, P^{#}, V^{#} or T^{#}
   X⁵ is selected from G^{#}, A^{#};
   wherein “^{#}” indicates that the listed residue is independently the unmodified amino acid, a modified amino acid or a non-natural amino acid analogue;
R² is NH₂, NR^{2a}H, NR^{2a}R^{2b}, OH or OR^{2a}; wherein each of R^{2a} and R^{2b} if present are independently C₁₋₆-alkyl (e.g. methyl, ethyl, propyl, butyl, pentyl or hexyl), C₃₋₆-alkenyl, C₆₋₁₆_aryl, C₆₋₁₆_aryl-C₁₋₆-alkyl, C₁₋₆-alkylene-C₆₋₁₆-aryl, or C₁₋₆_haloalkyl, each of which may optionally be substituted with one or more groups selected from halogen, C₁₋₆-alkyl, or C₁₋₆-haloalkyl.

In certain embodiments, Y¹ and Y² are both absent.

In another aspect, the invention provides an insecticidal compound having the formula (I) below:

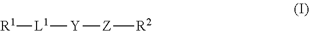

wherein:
R¹ is hydrogen (which may be designated “H-” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}—C(═N⁺(R^{1b})(R^{1c}))NR^{1d}R^{1e} acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀ aryl, biotin, a sugar moiety or (poly)alkyleneglycol, phosphate or sulphate;
wherein each of R^{1a}, R^{1b}, R^{1c}, R^{1d} and Rie is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl, a sugar moiety, phosphate or sulphate;
L¹ is absent or is:
   *—(C═O)C₁₋₁₆-alkylene-NH— where * denotes the point of attachment to Y or Z;
   (C₁₋₂₀)alkylene,
   (C₂₋₂₀)alkenylene,
   (poly)alkyleneglycol;
   wherein L¹ is optionally substituted with one or more groups selected from oxo (═O), halogen, ═N and ═S;
Y is absent or is a peptide comprising from 1 to 16 amino acids; wherein Y optionally comprises a (poly)alkyleneglycol linker in the peptide sequence;
Z is a peptide of the formula:

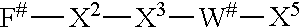

wherein:
   X² is selected from S^{#}, Y^{#}, K^{#}, N^{#} or H^{#};
   X³ is selected from S^{#}, A^{#}, P^{#}, V^{#} or T^{#}
   X⁵ is selected from G^{#}, A^{#};
   wherein “^{#}” indicates that the listed residue is independently the unmodified amino acid, a modified amino acid or a non-natural amino acid analogue;
R² is NH₂, NR^{2a}H, NR^{2a}R^{2b}, OH or OR^{2a}; wherein each of R^{2a} and R^{2b} if present are independently C₁₋₆-alkyl (e.g. methyl, ethyl, propyl, butyl, pentyl or hexyl), C₃₋₆-alkenyl, C₆₋₁₆_aryl, C₆₋₁₆_aryl-C₁₋₆-alkyl, C₁₋₆-alkylene-C₆₋₁₆-aryl, or C₁₋₆haloalkyl, each of which may optionally be substituted with one or more groups selected from halogen, C₁₋₆-alkyl, or C₁₋₆-haloalkyl.

Preferably:
R¹ is hydrogen (which may be designated “H-” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}, —C(═N⁺(R^{1b})(R^{1c}))NR^{1d}R^{1e} acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl, biotin, a sugar moiety or (poly)alkyleneglycol;
wherein each of R^{1a}, R^{1b}, R^{1c}, R^{1d} and R^{1e} is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moiety.

Preferably, X² is selected from S^{#}, Y^{#}, N^{#} or H^{#}.

Preferably, R² is NH₂, NR^{2a}H, NR^{2a}R^{2b} or OR^{2a}; wherein each of R^{2a} and R^{2b} if present are independently C₁₋₆-alkyl (e.g. methyl, ethyl, propyl, butyl, pentyl or hexyl), C₃₋₆-alkenyl, C₆₋₁₆_aryl, C₆₋₁₆_aryl-C₁₋₆-alkyl, C₁₋₆-alkylene-C₆₋₁₆-aryl, or C₁₋₆_haloalkyl, each of which may optionally be substituted with one or more groups selected from halogen, C₁₋₆-alkyl, or C₁₋₆-haloalkyl.

In another aspect, the invention provides an insecticidal compound having the formula (I) below:

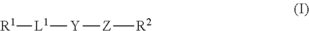

wherein:
R¹ is hydrogen (which may be designated “H-” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}—C(═N⁺(R^{1b})(R^{1c}))NR^{1d}R^{1e} acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀ aryl, biotin, a sugar moiety or (poly)alkyleneglycol;
wherein each of R^{1a}, R^{1b}, R^{1c}, R^{1d} and Rie is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moeity;
L¹ is absent or is:
   *—(C═O)C₁₋₆-alkylene-NH— where * denotes the point of attachment to Y or Z;
   (C₁₋₂₀)alkylene,
   (C₂₋₂₀)alkenylene,
   (poly)alkyleneglycol;
   wherein L¹ is optionally substituted with one or more groups selected from oxo (═O), halogen, ═N and ═S;
Y is absent or is a peptide comprising from 1 to 16 amino acids; wherein Y optionally comprises a (poly)alkyleneglycol linker in the peptide sequence;
Z is a peptide of the formula:

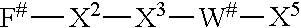

wherein:
   X² is selected from S^{#}, Y^{#}, N^{#} or H^{#};
   X³ is selected from S^{#}, A^{#}, P^{#}, V^{#} or T^{#},
   X⁵ is selected from G^{#}, A^{#};
   wherein “^{#}” indicates that the listed residue is independently the unmodified amino acid, a modified amino acid or a non-natural amino acid analogue;
R² is NH₂, NR^{2a}H, NR^{2a}R^{2b}, or OR^{2a}; wherein each of R^{2a} and R^{2b} if present are independently C₁₋₆-alkyl (e.g. methyl, ethyl, propyl, butyl, pentyl or hexyl), C₃₋₆-alkenyl, C₆₋₁₆_aryl, C₆₋₁₆_aryl-C₁₋₆-alkyl, C₁₋₆-alkylene-C₆₋₁₆-aryl, or C₁₋₆-haloalkyl, each of which may optionally be substituted with one or more groups selected from halogen, C₁₋₆-alkyl, or C₁₋₆-haloalkyl.

Suitably, Z is a peptide according to formula Z^{a}, wherein Z^{a} is a peptide of the formula:

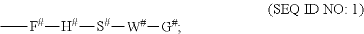

and wherein “^{#}” indicates that the listed residue is independently the unmodified amino acid, a modified amino acid or a non-natural amino acid analogue.

Suitably, Z is a peptide according to formula Z^{b}, wherein Z^{b} is a peptide of the formula:

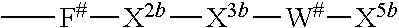

wherein:
X^{2b} is selected from amino acid residues S^{#}, Y^{#}, K^{#}, N^{#} or H^{#};
X^{3b} is selected from amino acid residues S^{#} A^{#}, P^{#}, V^{#} or T^{#};
X^{5b} is selected from amino acid residues A^{#} or G^{#}.
   and wherein “^{#}” indicates that the listed residue is independently the unmodified amino acid, a modified amino acid or a non-natural amino acid analogue.

Suitably, when Z is a peptide according to formula Z^{b}, X^{3b} is selected from amino acid residues S^{#} or A^{#}, wherein “^{#}” indicates that the listed residue is independently the unmodified amino acid, a modified amino acid or a non-natural amino acid analogue.

Suitably, Z is a peptide according to formula Z^{c}, wherein Z^{c} is a peptide of the formula:

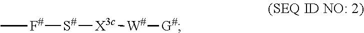

wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue.

Suitably, Y is absent or is a peptide comprising from 1 to 14 amino acids. Suitably, Y is absent or is a peptide comprising from 1 to 12 amino acids.

Suitably, compounds of Formula (I) (e.g. formulae (Ia), (Ib) and (Ic)) find particular use as insecticides against insects of the orders diptera, hemiptera, blattodea and/or lepidoptera.

Suitably, compounds of Formula (I) (e.g. formulae (Ia), (Ib) and (Ic)) find particular use as insecticides against insects of the orders diptera, hemiptera or lepidoptera.

Suitably, compounds of Formula (I) (e.g. formulae (Ia), (Ib) and (Ic)) find particular use as insecticides against insects of the order diptera (e.g. Drosophila species).

Suitably, compounds of Formula (I) (e.g. formulae (Ia), (Ib) and (Ic)) find particular use as insecticides against Drosophila species.

Suitably, compounds of Formula (I) (e.g. formulae (Ia), (Ib) and (Ic)) find particular use as insecticides against insects of the order hemiptera (e.g. Halyomorpha halys species or Myzus persicae species).

Suitably, compounds of Formula (I) (e.g. formulae (Ia), (Ib) and (Ic)) find particular use as insecticides against Halyomorpha halys species.

Suitably, compounds of Formula (I) (e.g. formulae (Ia), (Ib) and (Ic)) find particular use as insecticides against Myzus persicae species.

Suitably, compounds of Formula (I) (e.g. formulae (Ia), (Ib) and (Ic)) find particular use as insecticides against insects of the order lepidoptera (e.g. Plutella xylostella species or Spodeptera frugiperda species).

Suitably, compounds of Formula (I) (e.g. formulae (Ia), (Ib) and (Ic)) find particular use as insecticides against Plutella xylostella species.

Suitably, compounds of Formula (I) (e.g. formulae (Ia), (Ib) and (Ic)) find particular use as insecticides against Spodeptera frugiperda species.

Suitably, compounds of Formula (I) (e.g. formulae (Ia), (Ib) and (Ic)) find particular use as insecticides against insects of the order blattodea (e.g. Blattella germanica species).

Suitably, compounds of Formula (I) (e.g. formulae (Ia), (Ib) and (Ic)) find particular use as insecticides against Blattella germanica species.

Suitably, compounds of Formula (XX) find particular use against any of the species discussed above.

The compounds of Formula (I) (e.g. formulae (Ia), (Ib) and (Ic)) may therefore be applied to any of the methods or uses described herein.

The compounds of Formula (XX) and (I) (e.g. formulae (Ia), (Ib) and (Ic)) may therefore be applied to any of the methods or uses described herein.

Suitably, the compounds of Formula (XX) and (I) (e.g. formulae (Ia), (Ib) and (Ic)) comprise at least one modified amino acid or non-natural amino acid analogue, e.g. in the Y¹, Y, Y² and/or Z group. The at least one modified amino acid or non-natural amino acid analogue may be as defined anywhere herein.

The label “^{#}” indicates that the residue in question may be a modified version of the amino acid, or that the reside may be replaced by a non-natural amino acid analogue. For example, S^{#}, indicates that the reside may be serine, a modified serine (e.g. D-serine, beta-serine) or a non-natural amino acid (e.g. [Aib])

In another aspect, there is provided a composition comprising a compound as defined herein, or a salt thereof, in admixture with one or more solvents, carriers, diluents, adjuvants, preservatives, dispersants, emulsifying agents, or synergists. Suitably, the composition is an agricultural composition, the composition is an insect control composition or plant protection composition.

In another aspect, there is provided the use of a compound as defined herein, or a salt or solvate thereof, or a composition as defined herein, as an insect control agent. The use may be as an insecticide against insects that encode the kinin peptide. The use may be as an insecticide against hemipteran, dipteran, blattodea and/or lepidopteran insects, preferably hemipteran, dipteran and/or lepidopteran insects.

In another aspect, there is provided a method of insect mortality, the method comprising contacting an insect population with a compound as defined herein, or a salt thereof, or a composition as defined herein. Suitably, the insect is an insect that encodes the kinin peptide, such as hemipteran, dipteran, blattodea and/or lepidopteran insects, more suitably hemipteran, dipteran and/or lepidopteran insects.

In another aspect, there is provided a method of increasing hemipteran, dipteran and/or lepidopteran insect mortality, the method comprising contacting a hemipteran, dipteran and/or lepidopteran insect or hemipteran, dipteran and/or lepidopteran insect population with a compound as defined herein, or a salt thereof, or a composition as defined herein.

In another aspect, there is provided a method of increasing blattodea insect mortality, the method comprising contacting a blattodea insect or blattodea insect population with a compound as defined herein, or a salt thereof, or a composition as defined herein.

In another aspect, there is provided the use of a compound as defined herein, or a salt thereof, or a composition as defined herein, as a plant protection agent, for protecting a plant against insects that encode the kinin peptide.

In another aspect, there is provided the use of a compound as defined herein, or a salt thereof, or a composition as defined herein, as a plant protection agent, for protecting a plant against hemipteran, dipteran and/or lepidopteran insects.

In another aspect, there is provided the use of a compound as defined herein, or a salt thereof, or a composition as defined herein, as a plant protection agent, for protecting a plant against blattodea insects.

In another aspect, there is provided a method of inhibiting infestation of a plant by insects that encode the kinin peptide, the method comprising contacting the plant with a compound as defined herein, or a salt thereof, or a composition as defined herein. Suitably, the compound or composition is applied to the plant while the plant is free or substantially free of insects that encode the kinin peptide.

In another aspect, there is provided a method of inhibiting infestation of a plant by hemipteran, dipteran and/or lepidopteran insects comprising contacting the plant with a compound as defined herein, or a salt thereof, or a composition as defined herein. Suitably, the compound or composition is applied to the plant while the plant is free or substantially free of hemipteran, dipteran and/or lepidopteran insects.

In another aspect, there is provided a method of inhibiting infestation of a plant by blattodea insects comprising contacting the plant with a compound as defined herein, or a salt thereof, or a composition as defined herein. Suitably, the compound or composition is applied to the plant while the plant is free or substantially free of blattodea insects.

In another aspect, there is provided a method of reducing infestation of a plant by insects that encode the kinin peptide, or of reducing load of insects that encode the kinin peptide on a plant, the method comprising contacting the plant with a compound as defined herein, or a salt thereof, or a composition as defined herein.

In another aspect, there is provided a method of reducing hemipteran, dipteran and/or lepidopteran insect infestation of a plant, or of reducing hemipteran, dipteran and/or lepidopteran insect load on a plant, the method comprising contacting the plant with a compound as defined herein, or a salt thereof, or a composition as defined herein.

In another aspect, there is provided a method of reducing blattodea insect infestation of a plant, or of reducing blattodea insect load on a plant, the method comprising contacting the plant with a compound as defined herein, or a salt thereof, or a composition as defined herein.

Suitable insects which encode a kinin peptide include hemipteran, dipteran and/or lepidopteran insects. Other insects which may encode a kinin peptide are of the order blattodea.

In another aspect there is provided the use of a compound or a composition as defined herein as an insect control agent, or as a plant protection agent for protecting a plant or part thereof against insects.

In another aspect there is provided the use of a compound or a composition as defined herein against:
a) dipteran insects, preferably wherein the dipteran insects are selected from: D. melanogaster or D. suzukii;
b) hemipteran insects, such as aphids, preferably wherein the hemipteran insects are selected from: Aphis fabae, Acyrthosiphon pisum, Amrasca biguttula, Myzus persicae or Rhopalosiphum padi; and/or
c) lepidopteran insects, preferably wherein the lepidopteran insects are selected from: Plutella xylostella or Spodeptera frugiperda; and/or
d) blattodea insects (e.g. Blattella germanica).

In another aspect there is provided a method of increasing insect mortality, the method comprising contacting an insect or insect population with a compound or a composition as defined herein.

In another aspect there is provided a method of inhibiting or preventing infestation of a plant by insects, the method comprising contacting the plant or a part thereof, or a site on which the plant is growing or is intended to be grown, with a compound or a composition as defined herein. Suitably, the compound or composition is contacted with the plant or part thereof, or a site on which the plant is growing or is intended to be grown, while the plant or part is free or substantially free of insects.

In another aspect there is provided a method of reducing or treating an insect infestation of a plant, or of reducing insect load on a plant, the method comprising contacting the plant or a part thereof, or a site on which the plant is growing, with a compound or a composition as defined herein.

Suitably, the site on which the plant is growing or intended to be grown may comprise an agricultural site suitable for growing plants, preferably the site comprises a field.

Suitably, the insects are:
a) dipteran insects, preferably wherein the dipteran insects are selected from: D. melanogaster or D. suzukii;
b) hemipteran insects, such as aphids, preferably wherein the hemipteran insects are selected from Aphis fabae, Acyrthsiphon pisum, Myzus persicae or Rhopalosiphum padi;
c) lepidopteran insects, preferably wherein the lepidopteran insects are selected from: Plutella xylostella or Spodeptera frugiperda; and/or
d) blattodea insects, preferably wherein the blattodea insects are Blattella germanica.

Suitably, contacting the insect or insect population, plant or part thereof, or site, comprises watering feeding, spraying, atomizing, foaming, fogging, culturing in hydroculture, culturing in hydroponics, coating, submerging, injecting and/or encrusting the insect or insect population, plant or part thereof, or site with the composition as defined herein. Contacting may comprise injecting a plant (e.g. a tree) or part thereof with the composition as defined herein, using systems and methods such as those described in WO2020/021041, WO2023/161802, WO2022/264053, WO2022/189386, WO2022/165248, WO2021/152093, WO2020/212612 and WO2020/021041, the entire contents of which being incorporated by reference.

Suitably, the insect or insect population, plant or part thereof, or site, is contacted with an effective concentration of the compound as defined herein, preferably a concentration of between 10⁻³ to 10⁻⁹ M.

In another aspect, there is provided a method of producing an insecticidal compound according to the first aspect, the method comprising:
(a) chemically synthesising the insecticidal compound of formula (I) or (XX), or a precursor thereof.

In one embodiment, the chemical synthesis comprises the method described in the examples.

Further features of the aspects and embodiments of the invention will now be defined under the following headed sections. Any feature in any section may be combine with any aspect of embodiment in any workable combination.

### DETAILED DESCRIPTION OF THE INVENTION

### Definitions

Throughout the present description and claims the conventional three-letter and one-letter codes for naturally occurring amino acids are used, i.e. A (Ala), G (Gly), L (Leu), I (Ile), V (Val), F (Phe), W (Trp), S (Ser), T (Thr), Y (Tyr), N (Asn), Q (Gln), D (Asp), E (Glu), K (Lys), R (Arg), H (His), M (Met), C (Cys) and P (Pro).

Generally, herein if the one letter code is accompanied by a “#” symbol, it indicates that it may be either in its naturally occurring form, a modified form, or may be replaced with a non-proteinogenic amino acid. For Example, “A^{#}” encompasses unmodified alanine, a modified alanine (e.g. N-methyl alanine) or indicates that an alanine residue could be replaced by another non-natural amino acid analogue (e.g. α-aminoisobutyric acid (Aib)).

Suitably, in the F^{#}—H^{#}—S^{#}—W^{#}-G^{#} (SEQ ID NO: 1) motif, the residues may be in a naturally occurring form or a modified form.

Suitably, in the F^{#}—H^{#}—S^{#}—W^{#}-G^{#} (SEQ ID NO: 1) motif, a single residue may be replaced with a non-proteinogenic amino acid, e.g. FH[Aib]WG (SEQ ID NO: 3), where serine is replaced by α-aminoisobutyric acid (Aib).

Suitably, in the F^{#}—H^{#}—S^{#}—W^{#}-G^{#} (SEQ ID NO: 1) motif for example, a single residue may be replaced with a D-amino acid or an N-methylated amino acid e.g. FHsWG (SEQ ID NO: 4), where serine is replaced by D-serine, or FH[n-me-S]WG (SEQ ID NO: 5), wherein serine is replaced with N-methyl serine.

‘Amino acid’ as referred to herein may refer to a naturally occurring amino acid or any other amino acid including synthetic amino acids, and non-proteinogenic amino acids. By “naturally occurring” in this context is meant the 20 amino acids encoded by the standard genetic code, sometimes referred to as proteinogenic amino acids.

The term amino acid is short for α-amino [alpha-amino]carboxylic acid. Each molecule contains a central carbon (C) atom, called the α-carbon, to which both an amino and a carboxyl group are attached. The remaining two bonds of the α-carbon atom are generally satisfied by a hydrogen (H) atom and the side chain, shown as R below:

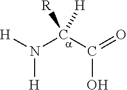

The modified amino acid in the present invention may be an α-alkylated amino acid, in which the α hydrogen atom has been replaced with a C₁₋₆alkyl group, e.g. a methyl or an ethyl group. Preferably, the α alkylated amino acid is an α-methylated amino acid, e.g. α-methyl-L-serine (given the notation [α-Me)S]):

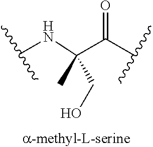 [SMILES: *N[C@@](C)(CO)C(*)=O]

Generally accepted three-letter codes and other abbreviations for other amino acids may also be employed, such as hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib), thiazolidine-4-carboxylic acid (Thz) etc.

The term “Thz” indicates that an amino acid residue has been replaced with a thiazolidine-4-carboxylic acid residue, for example (R)-thiazolidine-4-carboxylic acid:

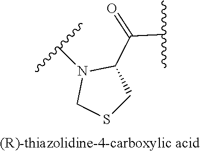 [SMILES: *C(=O)[C@@H]1CSCN1*]

The notation “Ahx” indicates 6-aminohexanoic acid (also known as 6-aminocaproic acid or ε-aminocaproic acid). “Ado” indicates 12-aminododecanoic acid.

The notation “Ava” indicates a 5-aminovaleric acid moiety, i.e.:

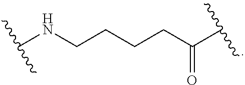 [SMILES: *NCCCCC(*)=O]

The notation “n-me” before an amino acid code is used to indicate an N-methylated amino acid residue. Thus, for example, “[n-me-V]” indicates N-methyl valine, “[n-me-A]” indicates N-methyl alanine and “[n-me-L]” indicates N-methyl leucine.

Such other amino acids may be shown in square brackets “[ ]” (e.g. “[Aib]”) when used in a general formula or sequence in the present specification, especially when the rest of the formula or sequence is shown using the single letter code.

Unless otherwise specified, amino acid residues in peptides of the invention are of the L-configuration. However, D-configuration amino acids may be incorporated as modifications. In the present context, an amino acid code written with a small letter may be used to represent the D-configuration of said amino acid, e.g. [a] represents the D-configuration of alanine.

Residues of beta amino acids may also be employed in compounds the invention. Such residues may be designated by a “β” symbol followed by the conventional code for the corresponding alpha amino acid. Thus, [βhL](or [B3L]) represents a residue of beta-homoleucine (3-amino-5-methylcaproic acid), [βA] represents a residue of beta-alanine (3-aminopropanoic acid), [βhA](or [B3A]) represents a residue of beta-homoalanine, [βhV] represents a residue of beta-homovaline, sometimes referred to as beta-leucine (3-amino-4-methylpentanoic acid), [βhF](or [B3F]) represents a residue of beta-homo-phenylalanine, [βhP](or [B3P]) represents a residue of beta-homoproline.

A “peptoid analogue” of an amino acid is an analogue in which the side chain is connected to the nitrogen of the peptide backbone, instead of the α-carbon as in proteinogenic peptides. Such residues may be designated by a “Peptoid” notation followed by the conventional code for the corresponding alpha amino acid. An example of a peptoid includes a peptoid of phenylalanine:

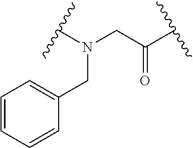 [SMILES: *C(=O)CN(*)Cc1ccccc1]

### A Peptoid of Phenylalanine

The above residue may also have the notation [Peptoid-F], indicating a peptoid of phenylalanine.

A “biotin modified” amino acid analogue is an amino acid residue which incorporates a biotin moiety as part of the side chain. For example, the notation “[Biotin-K]” indicates a lysine residue modified at the side chain to include a biotin moiety:

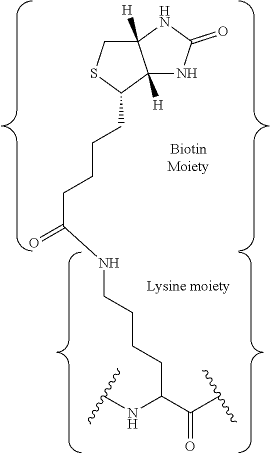

Alternatively, or in addition, a biotin moiety may be at a terminal position. Thus, R₁ may be biotin. The notation [Biotin]- represents a terminal biotin moiety, for example a partial sequence [Biotin]-S has the formula:

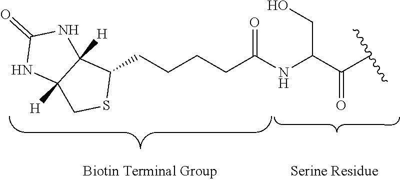 [SMILES: *C(=O)C(CO)NC(=O)CCCC[C@@H]1SC[C@@H]2NC(=O)N[C@@H]21]

The modified amino acid precursors used to fabricate the peptides disclosed herein may be commercially available or be synthesised by methods known in the art.

The notation Cₓ₋ₓₓ refers to the number of carbon atoms in a functional group. The number in the ‘x’ positions is the lowest number of carbon atoms and the number in the ‘xx’ position denotes the highest number of carbon atoms. For example, C₁₋₆-alkyl refers to alkyl groups as defined herein having from 1 to 6 carbon atoms.

The notation i, n or t are used herein in relation to various alkyl groups in the normal way. Specifically, the suffixes refer to the arrangement of atoms and denotes straight chain (‘n’) or branched (‘i’ or ‘t’) alkyl groups.

The term “alkyl” as used herein refers to a saturated linear or branched-chain monovalent hydrocarbon radical, wherein the alkyl radical may be optionally substituted.

The number of carbon atoms in the alkyl group may be specified using the above notation, for example, when there are from 1 to 8 carbon atoms the term “C₁₋₈-alkyl” may be used.

Examples of alkyl groups include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), and 1-butyl (n-Bu, n-butyl, —CH₂CH₂CH₂CH₃).

An “alkylene,” “alkenylene,” or “alkynylene” group is an alkyl, alkenyl, or alkynyl group that is positioned between and serves to connect two other chemical groups. Thus, “C₁₋₆alkylene” means a linear saturated divalent hydrocarbon radical of one to six carbon atoms or a branched saturated divalent hydrocarbon radical of three to six carbon atoms. Example alkylene groups include methylene (—CH₂—), 1,1-ethylene (—CH(CH₃)—), 1,2-ethylene (—CH₂CH₂—), 1,1-propylene (—CH(CH₂CH₃)—), and 2,2-propylene (—C(CH₃)₂—).

The term “alkenyl” as used herein refers to a linear or branched-chain monovalent hydrocarbon radical with at least one site of unsaturation, i.e., a carbon-carbon double bond.

The alkenyl radical may be optionally substituted, and includes radicals having “cis” and “trans” orientations, or alternatively, “E” and “Z” orientations. The number of carbon atoms in the alkenyl group may be specified using the above notation, for example, when there are from 2 to 8 carbon atoms the term “C₂₋₈-alkenyl” may be used. Example alkenyl groups include, but are not limited to, ethenyl (—CH═CH₂), and prop-1-enyl (—CH═CHCH₃).

In the chemical structures drawn herein, the presence of “ [symbol] ” or “ [symbol] ” denotes a point of attachment or a radical for example, a radical as discussed in relation to various functional groups.

The term “aryl” means a cyclic or polycyclic aromatic ring having from 5 to 12 carbon atoms. The term aryl includes both monovalent species and divalent species. Aryl includes groups having a single ring and groups having more than one ring such a fused rings or spirocycles. In the case of groups having more than one ring, at least one of the rings is aromatic. The number of carbon atoms in the aryl group may be specified using the above notation, for example, when there are from 6 to 16 carbon atoms the term “C₆₋₁₆-aryl” may be used. Aryl groups may be optionally substituted. Examples of aryl groups include phenyl, naphthyl, biphenyl, phenanthrenyl, naphthacenyl, 1,2,3,4-tetrahydronaphthalenyl, 1H-indenyl, 2,3-dihydro-1H-indenyl, and fluorenyl. A preferred aryl group is fluorenyl.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3-b]furanyl, 2H-furo[3,2-b]-pyranyl, 5H-pyrido[2,3-d]-o-oxazinyl, 1H-pyrazolo[4,3-d]-oxazolyl, 4H-imidazo[4,5-d]thiazolyl, pyrazino[2,3-d]pyridazinyl, imidazo[2,1-b]thiazolyl, imidazo[1,2-b][1,2,4]triazinyl. “Heteroaryl” also covers partially aromatic bi- or polycyclic ring systems wherein at least one ring is an aromatic ring and one or more of the other ring(s) is a non-aromatic, saturated or partially saturated ring, provided at least one ring contains one or more heteroatoms selected from nitrogen, oxygen or sulfur. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl and 3,4-dihydro-2H-pyrido[3,2-b][1,4]oxazinyl. A particularly preferred heteroaryl is indolyl, which may be designated “[Ind]”.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups. A preferred bicyclic heteroaryl group is indolyl.

Particular examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

The term halogen as used herein refers the one or more of fluorine (F), chlorine (Cl), bromine (Br) or iodine (I). The term “halo” or “halogeno” refers to fluoro, chloro, bromo and iodo.

The term “haloalkyl” refers to an alkyl group having on or more halogen substituent. The number of carbon atoms in the haloalkyl group may be specified using the above notation, for example, when there are from 1 to 8 carbon atoms the term “C₁₋₈-haloalkyl” may be used. Examples of haloalkyl groups include trifluoromethyl (—CF₃).

The term “comprising” is used in the present description and claims, it does not exclude other elements or steps. Where an indefinite or definite article is used when referring to a singular noun e.g. “a” or “an”, “the”, this includes a plural of that noun unless something else is specifically stated.

The term “about” as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

“Plant” as used herein, means an entire plant or a part thereof, including fresh fruit, vegetables and seeds. The plant or plant part may be a live plant or part thereof. Also, the term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

“Crop” as used herein means a plant species or variety that is grown to be harvested as food, livestock fodder, fuel raw material, or for any other economic purpose. As a non-limiting example, said crops can be maize, cereals, such as wheat, rye, barley and oats, sorghum, rice, sugar beet and fodder beet, fruit, such as pome fruit (e.g. apples and pears), citrus fruit (e.g. oranges, lemons, limes, grapefruit, or mandarins), stone fruit (e. g. peaches, nectarines or plums), nuts (e.g. almonds or walnuts), soft fruit (e.g. cherries, strawberries, blackberries or raspberries), the plantain family or grapevines, leguminous crops, such as beans, lentils, peas and soya, oil crops, such as sunflower, safflower, rapeseed, canola, castor or olives, cucurbits, such as cucumbers, melons or pumpkins, fibre plants, such as cotton, flax or hemp, fuel crops, such as sugarcane, miscanthus or switchgrass, vegetables, such as potatoes, tomatoes, peppers, lettuce, spinach, onions, carrots, egg-plants, asparagus or cabbage, ornamentals, such as flowers (e.g. petunias, pelargoniums, roses, tulips, lilies, or chrysanthemums), shrubs, broadleaved trees (e.g. poplars or willows) and evergreens (e.g. conifers), grasses, such as lawn, turf or forage grass or other useful plants, such as coffee, tea, tobacco, hops, pepper, rubber or latex plants.

A “pest”, as used here, is an organism that is harmful to plants, animals, humans or human concerns, and includes, but is not limited to crop pests such as insects (as later defined), household pests or insects, such as cockroaches, ants, etc., and disease vectors, such as malaria mosquitoes.

“Pest infection” or “pest disease” as used herein refers to any inflammatory condition, disease or disorder in a living organism, such as a plant, animal or human, which is caused by a pest.

“Active substance”, “active ingredient” or “active principle”, as used interchangeably herein, means any biological, biochemical or chemical element and its derivatives, fragments or compounds based thereon, including micro-organisms, having general or specific action against harmful organisms on a subject, and in particular on insect pests of plants, parts of plants or on plant products, as they occur naturally or by manufacture, including any impurity inevitably resulting from the manufacturing process.

The terms “effective amount” and “effective dose”, as used herein, mean the amount needed to achieve the desired result or results.

An “insect”, as used here, is used in the broad popular sense and includes all species of the superphylum Panarthropoda (classification Systema Naturae, Brands, S. J. (comp.) 1989-2005. Systema Naturae 2000. Amsterdam, The Netherlands, [http://sn2000.taxonomy.nl/]), including the phyla Arthropoda, Tardigrada and Onychophora; it includes all the different phases of the life cycle, such as, but not limited to eggs, larvae, nymphs, pupae and adults. Suitable insect pests are defined elsewhere herein.

An “insecticidal compound” as used herein refers to compounds having biological activity on insects (as defined above), including but not limited to compounds capable of killing the insect, larvaecides, insect growth regulators, behaviour modifying compounds, attractants, repellents, pheromones, kairomones, allomones and entomopathogenic fungi, viruses and proteins. The insecticide exerts its biological activity preferably by the contact of the compound with the insect, without the need of being ingested by the insect. It includes not only compounds or compound formulations that are ready to use, but also precursors in an inactive form, which may be activated by outside factors. Possibly, the insecticide may be combined with materials used in conjunction, such as synergists or safeners, flavour or odour compositions. Preferably, said compound is comprised in a carrier as defined above. “Comprised in a carrier” as used herein means bound on or contained in by means such as but not limited to embedding, encapsulation and adsorption.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### Further Definitions of the Compounds of the Invention

Particular compounds of the invention include, for example, compounds of the formula (I) or (XX), wherein, unless otherwise stated, each of R¹, L¹, Y, Y¹, Y², Z, R² and any associated substituent group has any of the meanings defined hereinbefore or in any of paragraphs (1) to (28) hereinafter:—
(1) R¹ is selected from:
   hydrogen
   —C(═N⁺Me₂)NMe₂,
   acyl,
   fatty acyl,
   benzyl,
   benzoyl,
   heteroaryl
   trifluoroacetyl,
   —NHC₁₋₁₈alkyl,
   —NHC₆₋₁₆Aryl,
   —NH—C₁₋₆alkyl-C₆₋₁₀aryl;
   a sugar moiety;
   biotin;
   (poly)alkyleneglycol (e.g. polyethylene glycol such as PEG-1 to PEG-16);
   wherein each of R^{1a}, R^{1b}, R^{1c}, R^{1d} and R^{1e} is independently selected from hydrogen or C₁₋₄ alkyl (e.g. methyl, ethyl, propyl, butyl);
   and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moiety;
(2) R¹ is selected from:
   hydrogen;
   acyl optionally substituted with a sugar moiety;
   fatty acyl;
   heteroaryl;
   —NHC₆₋₁₆Aryl;
   a sugar moiety;
   biotin; or
   (poly)ethyleneglycol of the formula *—(OCH₂CH₂)ₙ—Rₚ, where * denotes the point of attachment to L¹, Y¹, Y or Z, n is an integer from 1 to 16, and Rₚ is selected from —NH₂, —OH or —OMe.
(3) R¹ is selected from:
   hydrogen;
   acyl optionally substituted with a sugar moiety, wherein the acyl group is selected from formyl, acetyl (Ac), propanoyl, butanoyl;
   fatty acyl selected from palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl;
   heteroaryl selected from indolyl ([Ind]);
   —NHC₆₋₁₆Aryl;
   a monosaccharide or disaccharide moiety;
   biotin;
   (poly)ethyleneglycol of the formula *—(OCH₂CH₂)ₙ—Rₚ, where * denotes the point of attachment to L¹, Y¹, Y or Z, n is an integer from 4 to 12, and Rₚ is selected from —NH₂, —OH or —OMe.
(4) R¹ is selected from:
   hydrogen;
   acetyl optionally substituted with a monosaccharide or disaccharide moiety;
   palmitoyl;
   indolyl,
   (poly)ethyleneglycol of the formula *—(OCH₂CH₂)ₙ—Rₚ, where * denotes the point of attachment to L¹, Y¹, Y or Z, n is an integer from 6 to 10, and Rₚ is selected from —NH₂, -or OH.
(5) R¹ is selected from:
   hydrogen;
   acetyl;
   (poly)ethyleneglycol of the formula *—(OCH₂CH₂)₈—NH₂ (PEG8), where * denotes the point of attachment to L¹, Y¹, Y or Z.
(6) R¹ is hydrogen or acetyl.
(7) L¹ is absent or is:
   *—(C═O)C₁₋₁₀-alkylene-NH—, where * denotes the point of attachment to Z;
   C₁₋₁₀alkylene
   wherein L¹ is optionally substituted with one or more oxo (═O) groups.
(8) L¹ is absent or is:
   —(C═O)C₄₋₁₂-alkylene-NH—, where * denotes the point of attachment to L¹, Y¹, Y or Z;
   —(C═O)—C₁₋₄alkylene-(C═O)—.
(9) L¹ is absent or is:

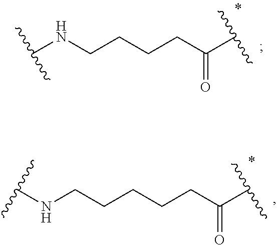 [SMILES: *NCCCCC(*)=O ; *NCCCCCC(*)=O]

where * denotes the point of attachment to L¹, Y¹, Y or Z; or

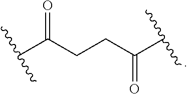 [SMILES: *C(=O)CCC(*)=O]

(10) L¹ is absent or is:

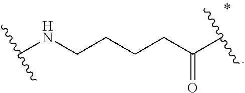 [SMILES: *NCCCCC(*)=O]

(11) In the moiety Y—Z, and sub formulae thereof, the one or more modified or non-natural amino acids, if present, are independently selected from:
   i. an N-methylated amino acid;
   ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
   iii. a D-amino acid;
   iv. an α-alkylated (e.g. α-methylated) amino acid;
   v. a beta-amino acid;
   vi. a peptoid amino acid analogue;
   vii. a sugar modified amino acid; or
   viii. a biotin modified amino acid.
(12) In the moiety Y—Z, and sub formulae thereof, the one or more modified or non-natural amino acids, if present, are independently selected from:
   i. an N-methylated amino acid;
   ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
   iii. an α-methylated amino acid;
   iv. a D-amino acid; or
   v. a beta amino acid.
(13) In the moiety Y—Z, and sub formulae thereof, the one or more modified or non-natural amino acids, if present, are independently selected from:
   i. an N-methylated amino acid;
   ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
   iii. an α-methylated amino acid;
   iv. a D-amino acid.
(14) In the moiety Y—Z, and sub formulae thereof, the one or more modified or non-natural amino acids, if present, are independently selected from:
   i. an N-methylated amino acid;
   ii. α-aminoisobutyric acid (Aib);
   iii. thiazolidine-4-carboxylic acid (Thz);
   iv. an α-methylated amino acid;
   v. a D-amino acid.
(15) In the moiety Y—Z, and sub formulae thereof, the one or more modified or non-natural amino acids, if present, are independently selected from:
   i. an N-methylated amino acid;
   ii. a D-amino acid.
(16) Y is absent or is a peptide comprising from 1 to 16 amino acids.
(17) Y is absent or is a peptide comprising from 1 to 12 (e.g. from 3 to 10) amino acids.
(18) Y is absent or is a peptide comprising from 1 to 8 amino acids.
(19) Z is a group Z^{a}, Z^{b} or Z^{c} as defined anywhere herein.
(20) Z is a group Z^{a} as defined anywhere herein.
(21) Z is a group Z^{b} as defined anywhere herein.
(22) Z is a group Z^{c} as defined anywhere herein.
(23) R² is NH₂, NR^{2a}H or NR^{2a}R^{2b}, wherein each of R^{2a} and R^{2b} if present is independently C₁₋₆-alkyl (e.g. methyl, ethyl, propyl, butyl, pentyl or hexyl).
(24) R₂ is NH₂;
(25) Y¹ is absent or a peptide comprising 1 or 2 amino acid residues.
(26) Y¹ is absent.
(27) Y² is absent or a peptide comprising 1 or 2 amino acid residues.
(28) Y² is absent.

Suitably, R¹ is as defined in any one of paragraphs (1) to (6) above. Most suitably, R¹ is as defined in any one of paragraphs (4) to (6) above.

Suitably, L¹ is as defined in any one of paragraphs (7) to (10) above. Most suitably, L¹ is as defined in paragraph (10) above.

Suitably, Y is as defined in any one of paragraphs (16) to (18), or is a group Y^{a}, Y^{b} or Y^{c} as defined herein.

Suitably, Z is as defined in any one of paragraphs (19) to (22).

Suitably, the at least one modified amino acid or non-natural amino acid analogue in peptide Y and/or Z is as defined in any one of paragraphs (11) to (15) above.

More suitably, the at least one modified amino acid or non-natural amino acid analogue in peptide Y and/or Z is as defined in any one of paragraphs (13) to (15) above. Most suitably, the at least one modified amino acid or non-natural amino acid analogue in peptide Y and/or Z is as defined in paragraph (14) or (15).

Suitably, R₂ is as defined in paragraph (23) or (24) above. Most suitably, R₂ is as defined in paragraph (24) above.

Suitably, Y¹ is as defined in any one of paragraphs (25) or (26) above. Most suitably, Y¹ is as defined in paragraphs (26) above.

Suitably, Y² is as defined in any one of paragraphs (27) or (28) above. Most suitably, Y² is as defined in paragraphs (28) above.

### Particular Peptides of the Invention

### Compounds of Formula (Ia)

The invention also relates to compounds of Formula (Ia) (a sub formula of Formula (I)) which find particular use against insects of the order diptera.

In a particular embodiment of the invention, the invention provides an insecticidal compound having the formula (Ia) below:

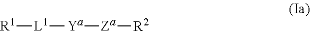

wherein:
   R¹, L¹, and R² are as defined herein;
   Y^{a} is a group Y as defined herein, optionally wherein Y^{a} is absent or is a peptide comprising from 1 to 12 amino acids;
   Z^{a} is a peptide of the formula:

(SEQ ID NO: 1)
-F^{#}-H^{#}-S^{#}-W^{#}-G^{#}

wherein “^{#}” indicates that the residues “F”, “H”, “S”, “W” and “G” are independently the unmodified amino acid, a modified amino acid or a non-natural amino acid analogue.

Suitably, at least one of the residues in the motif Y^{a}—Z^{a} is a modified amino acid or non-natural amino acid analogue.

Suitably, at least one of the residues in the motif Y^{a}—Z^{a} is a modified amino acid or non-natural amino acid analogue; with the proviso that if the S^{#} residue is an α-aminoisobutyric acid group ([Aib]), then the motif Y^{a}—Z^{a} further comprises at least one D-amino acid.

Suitably, the one or more modified amino acids or a non-natural amino acid analogues in the motif Y^{a}—Z^{a} are independently as defined anywhere herein. The one or more modified amino acids or a non-natural amino acid analogues are independently as defined in any one of paragraphs (11) to (15) herein. More suitably, the one or more modified amino acids or a non-natural amino acid analogues are independently as defined in any one of paragraphs (13), (14) or (15).

In an embodiment of the compounds of formula (Ia), the one or more modified amino acids or a non-natural amino acid analogues is independently selected from an N-methylated amino acid, aminoisobutyric acid (Aib) or a D-amino acid.

Suitably, the Z^{a} moiety is a peptide of the formula ZaI:

(SEQ ID NO: 6)
F-H^{#}-S^{#}-W-G (Zal)

wherein at least one of the “H” and “S” residues are a modified amino acid or non-natural amino acid analogue as defined herein.

Suitably, the Z^{a} moiety is a peptide of the formula ZaI-I:

(SEQ ID NO: 7)
F-H-S^{#}-W-G (Zal-I)

wherein the “S” residue is a modified amino acid or non-natural amino acid analogue as defined herein.

Suitably, the Z^{a} moiety is a peptide of the formula ZaI-I:

(SEQ ID NO: 8)
F-H-S^{#}-W-G

wherein the “S^{#}” residue is selected from α-aminoisobutyric acid, N-methyl serine or D-serine.

Suitably, the Z^{a} moiety is a peptide of the formula ZaI-I:

(SEQ ID NO: 9)
F-H-S^{#}-W-G

wherein the “S^{#}” residue is selected from N-methyl serine or D-serine.

Suitably, the Z^{a} moiety is a peptide of the formula:

(SEQ ID NO: 10)
FHSWG
(SEQ ID NO: 3)
FH[Aib]WG
(SEQ ID NO: 4)
FHsWG;
or
(SEQ ID NO: 5)
FH[n-me-S]WG.

Suitably, the Z^{a} moiety is a peptide of the formula:

(SEQ ID NO: 4)
FHsWG;
or
(SEQ ID NO: 5)
FH[n-me-S]WG.

Suitably, Y^{a} is absent or is a peptide containing from 1 to 12 amino acids, optionally comprising a PEG linker therein. More suitably, Y^{a} is a peptide containing from 1 to 12, e.g. from 3 to 10 amino acids.

Suitably, Y^{a} is absent or is a peptide of the formula:

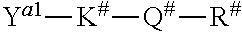

wherein Y^{a1} is absent or a peptide comprising from 1 to 9 amino acids; wherein “^{#}” indicates that the residue is either the naturally occurring amino acid, a modified amino acid or a non-natural amino acid analogue as defined herein.

Suitably, Y^{a} is absent or is a peptide of the formula:

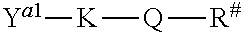

wherein Y^{a1} is absent or a peptide comprising from 1 to 9 amino acids; wherein the “R^{#}” residue is a modified amino acid or a non-natural amino acid analogue as defined herein.

Suitably, Y^{a} is absent or is a peptide of the formula:

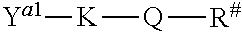

wherein Y^{a1} is absent or a peptide comprising from 1 to 9 amino acids; wherein the “R^{#}” residue is selected from N-methyl arginine or d-arginine.

Suitably, the Y^{a} moiety is absent or has the formula:
KQr;

(SEQ ID NO: 11)
NSVVLGKKQr;
or
(SEQ ID NO: 12)
NSVVLGKKQ[n-me-R].

Suitably, the Y^{a}—Z^{a} moiety is a peptide of the formula:

(SEQ ID NO: 13)
Y^{a1}-K^{#}-Q^{#}-R^{#}-F-H^{#}-S^{#}-W-G;
or
(SEQ ID NO: 138)
F-H#-S^{#}-W-G

wherein Y^{a1} is absent or a peptide comprising from 1 to 9 amino acids;
“^{#}” indicates that the residues “K”, “Q”, “R”, “H” and “S” are independently an unmodified amino acid, a modified amino acid or non-natural amino acid analogue;
wherein at least one of the residues “K”, “Q”, “R”, “H” and “S” is a modified amino acid or non-natural amino acid analogue as defined herein.

Suitably, the Y^{a}—Z^{a} moiety is a peptide of the formula:

(SEQ ID NO: 14)
Y^{a1}-K-Q-R^{#}-F-H^{#}-S^{#}-W-G;
or
(SEQ ID NO: 6)
F-H#-S^{#}-W-G

wherein Y^{a1} is absent or a peptide comprising from 1 to 9 amino acids;
wherein “^{#}” indicates that the residues “R”, “H” and “S” are independently an unmodified amino acid, a modified amino acid or non-natural amino acid analogue as defined herein;
with the proviso that at least one of the residues “R”, “H” and “S” is a modified amino acid or non-natural amino acid analogue as defined herein.

Suitably, the Y^{a}—Z^{a} moiety is a peptide of the formula:

(SEQ ID NO: 15)
Y^{a1}-K-Q-R^{#}-F-H-S^{#}-W-G;
or
(SEQ ID NO: 7)
F-H-S^{#}-W-G.

wherein Y^{a1} is absent or a peptide comprising from 1 to 9 amino acids;
wherein “^{#}” indicates that the residues “R”, and “S” are independently an unmodified amino acid, a modified amino acid or non-natural amino acid analogue;
with the proviso that at least one of the residues “R”, if present, and “S” is a modified amino acid or non-natural amino acid analogue as defined herein.

Suitably, the Y^{a}—Z^{a} moiety is a peptide of the formula:

(SEQ ID NO: 16)
Y^{a1}-K-Q-R^{#}-F-H-S^{#}-W-G;
or
(SEQ ID NO: 7)
F-H-S^{#}-W-G.

wherein Y^{a1} is absent or a peptide comprising from 1 to 9 amino acids;
wherein “^{#}” indicates that the residues “R”, if present, and “S” are independently selected from a modified amino acid or non-natural amino acid analogue as defined herein.

Suitably, the Y^{a}—Z^{a} moiety is a peptide of the formula:

(SEQ ID NO: 17)
Y^{a1}-K-Q-R^{#}-F-H-S^{#}-W-G;
or
(SEQ ID NO: 9)
F-H-S^{#}-W-G.

wherein Y^{a1} is absent or a peptide comprising from 1 to 9 amino acids;
wherein “^{#}” indicates that the residues “R”, and “S” are independently selected from the d-amino acid or an N-methylated amino acid,
   i.e. the R^{#} residue is selected from N-methyl arginine or d-arginine; and
      the S^{#} residue is selected from N-methyl serine or D-serine.

Particular compounds of Formula (Ia) include the compounds in the table below, or salts thereof:

| Reference | | |
|---|---|---|
| Number | Sequence | SEQ ID NO |
| SB-P-032 | Ac-KQrFH[Aib]WG-[NH₂] | SEQ ID NO: 18 |
| SB-P-033 | Ac-NSVVLGKKQrFH[Aib]WG- | SEQ ID NO: 19 |
| | [NH₂] | |
| SB-P-034 | Ac-FH[Aib]WG-[NH₂] | SEQ ID NO: 20 |
| SB-P-035 | Ac-KQrFHsWG-[NH₂] | SEQ ID NO: 21 |
| SB-P-036 | Ac-FHsWG-[NH₂] | SEQ ID NO: 22 |
| SB-P-052 | NSVVLGKKQ[n-me-R]FH[n- | SEQ ID NO: 23 |
| | me-S]WG-[NH₂] | |

### Particular Applications of Compounds of Formula (Ia)

Suitably, compounds of Formula (Ia) find particular use as insecticides against insects of the order diptera (e.g. Drosophila insects). Such compounds may therefore be applied in the appropriate methods defined herein.

There is also provided the use as an insect control agent, of a compound according to Formula (Ia) as defined herein. Suitably, said use is as an insecticide against insects of the order diptera (e.g. Drosophila insects).

There is also provided a method of increasing dipteran insect mortality, the method comprising contacting a dipteran insect or dipteran insect population with a compound according Formula (Ia) as defined herein, or a composition comprising a compound according to Formula (Ia) as defined herein.

There is also provided a compound according to Formula (Ia) as defined herein, or a composition comprising a compound according to Formula (Ia) as defined herein, as a plant protection agent, for protecting a plant against dipteran insects.

There is also provided a method of inhibiting infestation of a plant by dipteran insects comprising contacting the plant with a compound according to Formula (Ia) as defined herein, or a composition comprising a compound according to Formula (Ia) as defined herein. Suitably, the compound is applied to the plant while the plant is free or substantially free of dipteran insects.

In a particular embodiment, compounds of Formula (Ia) find particular use as insecticides against dipteran insects (e.g. D. suzukii), when the Z^{a} moiety is a peptide of the formula ZaI:

(SEQ ID NO: 6)
F-H^{#}-S^{#}-W-G (Zal)

wherein at least one of the “H” and “S” residues are a modified amino acid or non-natural amino acid analogue as defined herein.

In a particular embodiment, compounds of Formula (Ia) find particular use as insecticides against dipteran insects (e.g. D. suzukii), when the Z^{a} moiety is a peptide of the formula ZaI-I:

(SEQ ID NO: 7)
F-H-S^{#}-W-G (Zal-I)

wherein the “S” residue is a modified amino acid or non-natural amino acid analogue as defined herein. Suitably, the “S^{#}” residue is selected from α-aminoisobutyric acid, N-methyl serine or D-serine.

Although the compounds of Formula (Ia), and sub formulae thereof, particular use against insects of the type diptera, they may also be applied against any of the insect orders or species described in the methods and uses described herein, e.g. lepidoptera or hemiptera.

Suitably, the compounds of Formula (Ia) may be used in combination with one or more additional insecticides described herein.

In an embodiment, the compounds of Formula (Ia) may be used as an insect control agent against insects of the type dipteran in combination with one or more additional insecticides described herein.

### Compounds of Formula (Ib)

The invention also relates to compounds of Formula (Ib) (a sub formula of Formula (I)), which find particular use against insects of the order hemiptera (for example Halyomorpha halys and Myzus persicae species).

In an embodiment, the invention provides an insecticidal compound having the formula (Ib) below:

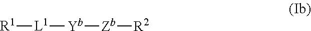

wherein:
R¹, L¹, and R² are as defined herein
Y^{b} is a group Y as defined herein, optionally wherein Y^{b} is absent or is a peptide comprising from 1 to 14 amino acids;
Z^{b} is a peptide of the formula:

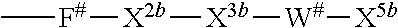

wherein:
X^{2b} is selected from amino acid residues S^{#}, Y^{#}, K^{#}, N^{#} or H #
X^{3b} is selected from amino acid residues S^{#}, A^{#}, P^{#}, V^{#} or T^{#};
X^{5b} is selected from amino acid residues A^{#} or G^{#},
wherein “^{#}” indicates that the listed residue is independently the unmodified amino acid, a modified amino acid or a non-natural amino acid analogue.

Suitably, Z^{b} is a peptide of the formula:

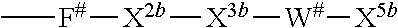

wherein:
X^{2b} is selected from amino acid residues S^{#}, Y^{#}, N^{#} or H #
X^{3b} is selected from amino acid residues S^{#}, A^{#}, P^{#}, V^{#} or T^{#};
X^{5b} is selected from amino acid residues A^{#} or G^{#},
wherein “^{#}” indicates that the listed residue is independently the unmodified amino acid, a modified amino acid or a non-natural amino acid analogue.

Suitably, Z^{b} is a peptide of the formula:

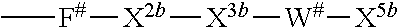

wherein:
X^{2b} is selected from amino acid residues S^{#}, Y^{#} N^{#} or H #
X^{3b} is selected from amino acid residues S^{#} or A^{#}, P^{#}, V^{#} or T^{#};
X^{5b} is selected from amino acid residues A^{#} or G^{#},
wherein “^{#}” indicates that the listed residue is independently the unmodified amino acid, a modified amino acid or a non-natural amino acid analogue.

More suitably, Z^{b} is a peptide of the formula:

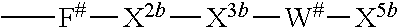

wherein:
   X^{2b} is selected from amino acid residues S^{#}, Y^{#} or N #
   X^{3b} is selected from amino acid residues S^{#} or A^{#},
   X^{5b} is selected from amino acid residues A^{#} or G^{#},
   wherein “^{#}” indicates that the listed residue is independently the unmodified amino acid, a modified amino acid or a non-natural amino acid analogue.

Suitably, Z^{b} is a peptide of the formula:

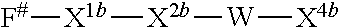

wherein:
X^{2b} is selected from amino acid residues S, Y or N;
X^{3b} is selected from amino acid residues S^{#} or A^{#}; and
X^{5b} is selected from amino acid residues A or G.

Suitably, Z^{b} is a peptide of the formula:

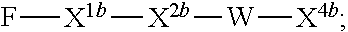

wherein:
   X^{2b} is selected from amino acid residues S, Y or N;
   X^{3b} is selected from amino acid residues S^{#} or A^{#}; and
   X^{5b} is selected from amino acid residues A or G.

Suitably, Z^{b} is a peptide with a formula selected from any one of (ZbI) to (ZbVIII) (sub formulae of Z^{b}):

(ZbI)
(SEQ ID NO: 24)
F^{#}-S^{#}-S^{#}-W^{#}-G^{#};
(ZbII)
(SEQ ID NO: 25)
F^{#}-S^{#}-S^{#}-W^{#}-A^{#};
(ZbIII)
(SEQ ID NO: 26)
F^{#}-N^{#}-S^{#}-W^{#}-A^{#};
(ZbIV)
(SEQ ID NO: 27)
F^{#}-Y^{#}-S^{#}-W^{#}-G^{#};
(ZbV)
(SEQ ID NO: 28)
F^{#}-N^{#}-A^{#}-W^{#}-A^{#};
(ZbVI)
(SEQ ID NO: 29)
F^{#}-S^{#}-A^{#}-W^{#}-G^{#};
(ZbVII)
(SEQ ID NO: 30)
F^{#}-N^{#}-S^{#}-W^{#}-G^{#};
or
(ZbVIII)
(SEQ ID NO: 31)
F^{#}-K^{#}-S^{#}-W^{#}-G^{#};

wherein any residue marked with a “^{#}” is independently either the listed unmodified amino acid, a modified amino acid or a non-natural amino acid analogue.

Suitably, Z^{b} is a peptide with a formula selected from any one of (ZbI-I) to (ZbVI-III-I) (sub formulae of Z^{b}):

(ZbI-I)
(SEQ ID NO: 32)
F^{#}-S-S^{#}-W-G;
(ZbII-I)
(SEQ ID NO: 33)
F^{#}-S-S^{#}-W-A;
(ZbIII-I)
(SEQ ID NO: 34)
F^{#}-N-S^{#}-W-A;
(ZbIV-I)
(SEQ ID NO: 35)
F^{#}-Y-S^{#}-W-G;
(ZbV-I)
(SEQ ID NO: 36)
F^{#}-N-A^{#}-W-A;
(ZbVI-I)
(SEQ ID NO: 37)
F^{#}-S-A^{#}-W-G;
(ZbVII-I)
(SEQ ID NO: 38)
F^{#}-N-S^{#}-W-G;
or
(ZbVIII-I)
(SEQ ID NO: 39)
F^{#}-K-S^{#}-W-G;

wherein any residue marked with a “^{#}” is independently either the listed unmodified amino acid, a modified amino acid or a non-natural amino acid analogue.

Suitably, in Formulae (ZbI-I) to (ZbVIII-I), the modified or non-natural amino acid analogues may be as defined anywhere herein.

In a particular embodiment, in Formulae (ZbI-I) to (ZbVIII-I), the modified or non-natural amino acid analogues are selected from an N-methylated amino acid, an α-methylated amino acid, aminoisobutyric acid (Aib), a D-amino acid or a beta-amino acid.

In a particular embodiment, in the compounds Formula (Ib):
Z^{b} is a peptide of the formula (ZbI) to (ZbVIII) or (ZbI-I) to (ZbVIII-I); and the residue marked F^{#} is selected from the unmodified amino acid F (phenylalanine) or is N-methylated phenylalanine.

In a particular embodiment, in the compounds Formula (Ib):
Z^{b} is a peptide of the formula (ZbI) to (ZbIV) or (ZbI-I) to (ZbIV-I); and the residue marked S^{#} is selected from the unmodified amino acid S (serine), n-methylated serine, beta-serine, alpha-methyl serine, or D-serine.

Suitably, Z^{b} is a peptide with a formula selected from any one of (ZbI) to (ZbVI) (sub formulae of Z^{b}):

(ZbI)
(SEQ ID NO: 24)
F^{#}-S^{#}-S^{#}-W^{#}-G^{#};
(ZbII)
(SEQ ID NO: 25)
F^{#}-S^{#}-S^{#}-W^{#}-A^{#};
(ZbIII)
(SEQ ID NO: 26)
F^{#}-N^{#}-S^{#}-W^{#}-A^{#};
(ZbIV)
(SEQ ID NO: 27)
F^{#}-Y^{#}-S^{#}-W^{#}-G^{#};
(ZbV)
(SEQ ID NO: 28)
F^{#}-N^{#}-A^{#}-W^{#}-A^{#};
or
(ZbVI)
(SEQ ID NO: 29)
F^{#}-S^{#}-A^{#}-W^{#}-G^{#};

wherein any residue marked with a “^{#}” is independently either the listed unmodified amino acid, a modified amino acid or a non-natural amino acid analogue.

Suitably, Z^{b} is a peptide with a formula selected from any one of (ZbI-I) to (ZbVI-I) (sub formulae of Z^{b}):

(ZbI-I)
(SEQ ID NO: 32)
F^{#}-S-S^{#}-W-G;
(ZbII-I)
(SEQ ID NO: 33)
F^{#}-S-S^{#}-W-A;
(ZbII-I)
(SEQ ID NO: 34)
F^{#}-N-S^{#}-W-A;
(ZbIV-I)
(SEQ ID NO: 35)
F^{#}-Y-S^{#}-W-G;
(ZbV-I)
(SEQ ID NO: 36)
F^{#}-N-A^{#}-W-A;
or
(ZbVI-I)
(SEQ ID NO: 37)
F^{#}-S-A^{#}-W-G;

wherein any residue marked with a “^{#}” is independently either the listed unmodified amino acid, a modified amino acid or a non-natural amino acid analogue.

Suitably, in Formulae (ZbI-I) to (ZbVI-I), the modified or non-natural amino acid analogues may be as defined anywhere herein.

In a particular embodiment, in Formulae (ZbI-I) to (ZbVI-I), the modified or non-natural amino acid analogues are selected from an N-methylated amino acid, an α-methylated amino acid, aminoisobutyric acid (Aib), a D-amino acid or a beta-amino acid.

In a particular embodiment, in the compounds Formula (Ib):
Z^{b} is a peptide of the formula (ZbI) to (ZbVI) or (ZbI-I) to (ZbVI-I); and the residue marked F^{#} is selected from the unmodified amino acid F (phenylalanine) or is N-methylated phenylalanine.

In a particular embodiment, in the compounds Formula (Ib):
Z^{b} is a peptide of the formula (ZbI) to (ZbIV) or (ZbI-I) to (ZbIV-I); and the residue marked S^{#} is selected from the unmodified amino acid S (serine), n-methylated serine, beta-serine, alpha-methyl serine, or D-serine

In another particular embodiment, in the compounds Formula (Ib):
Z^{b} is a peptide of the formula (ZbI) or (ZbI-I);
the residue marked F^{#} is the unmodified amino acid F (phenylalanine) or is N-methylated phenylalanine; and
S^{#} is the unmodified amino acid S (serine), n-methylated serine, beta-serine, alpha-methyl serine, or D-serine.

Suitably, Z^{b} is selected from:

(SEQ ID NO: 40)
FSSWG
(SEQ ID NO: 41)
FSSWA
(SEQ ID NO: 42)
FNSWA
(SEQ ID NO: 43)
FYAWG
(SEQ ID NO: 44)
FN[Aib]WA
(SEQ ID NO: 45)
FN-βA-WA
(SEQ ID NO: 46)
(n-me-F)-SsWG
(SEQ ID NO: 47)
(n-me-F)-S-βA-WG
(SEQ ID NO: 48)
(n-me-F)-S-(α-Me)S-WG;
(SEQ ID NO: 49)
FNSWG;
or
(SEQ ID NO: 50)
FKSWG.

Suitably, Z^{b} is selecte from:

(SEQ ID NO: 40)
FSSWG
(SEQ ID NO: 41)
FSSWA
(SEQ ID NO: 42)
FNSWA
(SEQ ID NO: 43)
FYAWG
(SEQ ID NO: 44)
FN[Aib]WA
(SEQ ID NO: 45)
FN-βA-WA
(SEQ ID NO: 46)
(n-me-F)-SsWG
(SEQ ID NO: 47)
(n-me-F)-S-βA-WG
(SEQ ID NO: 48)
(n-me-F)-S-(α-Me)S-WG.

In an embodiment, at least one of the residues in the motif Y^{b}—Z^{b} is a modified amino acid or non-natural amino acid analogue.

Suitably, the one or more modified amino acids or a non-natural amino acid analogues in the motif Y^{b}—Z^{b} are independently as defined anywhere herein. The one or more modified amino acids or a non-natural amino acid analogues are independently as defined in any one of paragraphs (11) to (15) herein. More suitably, the one or more modified amino acids or a non-natural amino acid analogues are independently as defined in any one of paragraphs (13), (14) or (15).

In an embodiment of the compounds of formula (Ib), the one or more modified amino acids or a non-natural amino acid analogues, if present, are independently selected from an N-methylated amino acid, aminoisobutyric acid (Aib), a D-amino acid or a beta-amino acid.

Suitably, Y^{b} is absent or is a peptide comprising from 1 to 14 amino acids. More suitably, Y^{b} is a peptide comprising from 1 to 12 amino acids.

Suitably, Y^{b} optionally comprises one or more modified amino acid or a non-natural amino acid analogue. Suitably, the one or more modified amino acid or a non-natural amino acid analogue present in Y^{b} is a d-amino acid.

Suitably, Y^{b} is selected from:

(SEQ ID NO: 51)
DPKYK;

AK;
AR;
SK;
VP;
PI;
GPD;
Sk;

(SEQ ID NO: 52)
RQKTV;

PA;

(SEQ ID NO: 53)
ASDKHGRPKQT;

Y;
GPA;
AAA;
NAA;
PA;
VPA;

(SEQ ID NO: 54)
TNTA;

PR;
GAD;
DPA;
SSA;

(SEQ ID NO: 55)
RAFTSTGSSRSPA;

PAS;
DAG;
SSG;
DPV;
DAA;
GSG;

(SEQ ID NO: 56)
DDDSDVSESGSK;

S;
G;

(SEQ ID NO: 57)
LDRSEGRVSKAL;
(SEQ ID NO: 58)
WGQSSVGAV;

GAE.

Suitably, Y^{b} is selected from:

(SEQ ID NO: 51)
DPKYK;

AK;
AR;
SK;
VP;
PI;
GPD;
Sk;

(SEQ ID NO: 52)
RQKTV;

PA; or

(SEQ ID NO: 53)
ASDKHGRPKQT.

Particular compounds of Formula (Ib) include the compounds in the table below, or salts or solvates thereof:

| Reference | | |
|---|---|---|
| number | Sequence | SEQ ID NO |
| SB-P-003 | Hy-DPKYKFSSWG-[NH₂] | SEQ ID NO: 59 |
| SB-P-004 | Hy-AKFSSWG-[NH₂] | SEQ ID NO: 60 |
| SB-P-018 | Hy-ARFSSWA-[NH₂] | SEQ ID NO: 61 |
| SB-P-019 | Hy-SKFNSWA-[NH₂] | SEQ ID NO: 62 |
| SB-P-020 | Hy-AKFSSWA-[NH₂] | SEQ ID NO: 63 |
| SB-P-021 | Hy-ARFNSWA-[NH₂] | SEQ ID NO: 64 |
| SB-P-022 | Hy-VPFNSWA-[NH₂] | SEQ ID NO: 65 |
| SB-P-023 | Hy-PIFSSWG-[NH₂] | SEQ ID NO: 66 |
| SB-P-024 | Hy-GPDFYAWG-[NH₂] | SEQ ID NO: 67 |
| SB-P-054 | Ac-SkFN-Aib-WA-[NH₂] | SEQ ID NO: 68 |
| SB-P-056 | Ac-SK-[n-me-F]-N-(α-Me)Ser-WA-[NH₂] | SEQ ID NO: 69 |
| SB-P-060 | Ac-PA-[n-me-F]-SsWG-[NH₂] | SEQ ID NO: 70 |
| SB-P-065 | Hy-RQKTVFSSWG-[NH₂] | SEQ ID NO: 71 |
| SB-P-066 | Hy-PAFSSWG-[NH₂] | SEQ ID NO: 72 |
| SB-P-067 | Hy-ASDKHGRPKQTFSSWG-[NH₂] | SEQ ID NO: 73 |
| SB-P-100 | Ac-PA-[n-me-F]-S-[βA]-WG-[NH₂] | SEQ ID NO: 74 |
| SB-P-101 | Ac-PA-[n-me-F]-S-(α-Me)S-WG-[NH₂] | SEQ ID NO: 75 |
| SB-P-143 | Hy-DPAFNSWG-[NH₂] | SEQ ID NO: 76 |
| SB-P-144 | Hy-SSAFNSWG-[NH₂] | SEQ ID NO: 77 |
| SB-P-145 | Hy-RAFTSTGSSRSPAFSSWG-[NH₂] | SEQ ID NO: 78 |
| SB-P-146 | Hy-PASFSSWG-[NH₂] | SEQ ID NO: 79 |
| SB-P-147 | Hy-DAGFSSWG-[NH₂] | SEQ ID NO: 80 |
| SB-P-148 | Hy-SSGFSSWG-[NH₂] | SEQ ID NO: 81 |
| SB-P-149 | Hy-DPVFKSWG-[NH₂] | SEQ ID NO: 82 |
| SB-P-150 | Hy-DAAFSSWG-[NH₂] | SEQ ID NO: 83 |
| SB-P-151 | Hy-GSGFSSWG-[NH₂] | SEQ ID NO: 84 |
| SB-P-154 | Hy-DDDSDVSESGSKFSSWG-[NH₂] | SEQ ID NO: 85 |
| SB-P-155 | Hy-SFSSWG-[NH₂] | SEQ ID NO: 86 |
| SB-P-156 | Hy-GFSSWG-[NH₂] | SEQ ID NO: 87 |
| SB-P-157 | Hy-LDRSEGRVSKALFSSWG-[NH₂] | SEQ ID NO: 88 |
| SB-P-158 | Hy-WGQSSVGAVFSSWG-[NH₂] | SEQ ID NO: 89 |
| SB-P-159 | Hy-GAEFYSWG-[NH₂] | SEQ ID NO: 90 |
| SB-P-169 | Hy-FSSWG-[NH₂] | SEQ ID NO: 91 |
| N/A | Hy-GPAFSSWG-[NH₂] | SEQ ID NO: 92 |
| N/A | Hy-AAAFNSWG-[NH₂] | SEQ ID NO: 93 |
| N/A | Hy-NAAFSSWG-[NH₂] | SEQ ID NO: 94 |
| N/A | Hy-NAAFNSWG-[NH₂] | SEQ ID NO: 95 |
| N/A | Hy-VPAFNSWG-[NH₂] | SEQ ID NO: 96 |
| N/A | Hy-TNTAFSSWG-[NH₂] | SEQ ID NO: 97 |
| N/A | Hy-PRFYSWG-[NH₂] | SEQ ID NO: 98 |
| N/A | Hy-GADFYAWG-[NH₂] | SEQ ID NO: 99 |
| N/A | [Ac]-SK[n-me-F]N[βAla]WA-[NH₂] | SEQ ID NO: 100 |

Particular compounds of Formula (Ib) include the compounds in the table below, or salts thereof:

| Reference | | |
|---|---|---|
| number | Sequence | SEQ ID NO |
| SB-P-003 | Hy-DPKYKFSSWG-[NH₂] | SEQ ID NO: 59 |
| SB-P-004 | Hy-AKFSSWG-[NH₂] | SEQ ID NO: 60 |
| SB-P-018 | Hy-ARFSSWA-[NH₂] | SEQ ID NO: 61 |
| SB-P-019 | Hy-SKFNSWA-[NH₂] | SEQ ID NO: 62 |
| SB-P-020 | Hy-AKFSSWA-[NH₂] | SEQ ID NO: 63 |
| SB-P-021 | Hy-ARFNSWA-[NH₂] | SEQ ID NO: 64 |
| SB-P-022 | Hy-VPFNSWA-[NH₂] | SEQ ID NO: 65 |
| SB-P-023 | Hy-PIFSSWG-[NH₂] | SEQ ID NO: 66 |
| SB-P-024 | Hy-GPDFYAWG-[NH₂] | SEQ ID NO: 67 |
| SB-P-054 | Ac-SkFN-Aib-WA-[NH₂] | SEQ ID NO: 68 |
| SB-P-056 | Ac-SK-[n-me-F]-N-(α-Me)Ser-WA-[NH₂] | SEQ ID NO: 69 |
| SB-P-060 | Hy-Ac-PA-[n-me-F]-SsWG-[NH₂] | SEQ ID NO: 70 |
| SB-P-065 | Hy-RQKTVFSSWG-[NH₂] | SEQ ID NO: 71 |
| SB-P-066 | Hy-PAFSSWG-[NH₂] | SEQ ID NO: 72 |
| SB-P-067 | Hy-ASDKHGRPKQTFSSWG-[NH₂] | SEQ ID NO: 73 |
| SB-P-100 | Ac-PA-[n-me-F]-S-[βA]-WG-[NH₂] | SEQ ID NO: 74 |
| SB-P-101 | Ac-PA-[n-me-F]-S-(α-Me)S-WG-[NH₂] | SEQ ID NO: 75 |

### Particular Applications of Compounds of Formula (Ib)

Suitably, compounds of Formula (Ib) find particular use as insecticides against insects of the order hemiptera (for example Halyomorpha halys and Myzus persicae species). Such compounds may therefore be applied in the appropriate methods defined herein.

The compounds of Formula (Ib) may find particular use as insecticides against Halyomorpha halys species.

The compounds of Formula (Ib) may find particular use as insecticides against aphid species.

The compounds of Formula (Ib) may find particular use as insecticides against Myzus persicae species.

In a particular embodiment, compounds of Formula (Ib) find particular use as insecticides against hemiptera insects (e.g. Halyomorpha halys species) when the Z^{b} moiety is a peptide of the formula selected from any one of (ZbI) to (ZbVIII) (sub formulae of Z^{b}):

(ZbI)
(SEQ ID NO: 24)
F^{#}-S^{#}-S^{#}-W^{#}-G^{#};
(ZbII)
(SEQ ID NO: 25)
F^{#}-S^{#}-S^{#}-W^{#}-A^{#};
(ZbIII)
(SEQ ID NO: 26)
F^{#}-N^{#}-S^{#}-W^{#}-A^{#};
(ZbIV)
(SEQ ID NO: 27)
F^{#}-Y^{#}-S^{#}-W^{#}-G^{#};
(ZbV)
(SEQ ID NO: 28)
F^{#}-N^{#}-A^{#}-W^{#}-A^{#};
(ZbVI)
(SEQ ID NO: 29)
F^{#}-S^{#}-A^{#}-W^{#}-G^{#};
(ZbVII)
(SEQ ID NO: 30)
F^{#}-N^{#}-S^{#}-W^{#}-G^{#};
or
(ZbVIII)
(SEQ ID NO: 31)
F^{#}-K^{#}-S^{#}-W^{#}-G^{#};

wherein any residue marked with a “^{#}” is independently either the listed unmodified amino acid, a modified amino acid or a non-natural amino acid analogue.

In a particular embodiment, compounds of Formula (Ib) find particular use as insecticides against hemiptera insects (e.g. Halyomorpha halys species) when the Z^{b} moiety is a peptide of the formula selected from any one of (ZbI) to (ZbVI) (sub formulae of Z^{b}):

(ZbI)
(SEQ ID NO: 24)
F^{#}-S^{#}-S^{#}-W^{#}-G^{#};
(ZbII)
(SEQ ID NO: 25)
F^{#}-S^{#}-S^{#}-W^{#}-A^{#};
(ZbIII)
(SEQ ID NO: 26)
F^{#}-N^{#}-S^{#}-W^{#}-A^{#};
(ZbIV)
(SEQ ID NO: 27)
F^{#}-Y^{#}-S^{#}-W^{#}-G^{#};
(ZbV)
(SEQ ID NO: 28)
F^{#}-N^{#}-A^{#}-W^{#}-A^{#};
or
(ZbVI)
(SEQ ID NO: 29)
F^{#}-S^{#}-A^{#}-W^{#}-G^{#};

wherein any residue marked with a “^{#}” is independently either the listed unmodified amino acid, a modified amino acid or a non-natural amino acid analogue.

In a particular embodiment, compounds of Formula (Ib) find particular use as insecticides against hemiptera insects (e.g. Halyomorpha halys species) when the Z^{b} is a peptide with a formula selected from any one of (ZbI-I) to (ZbV-I) (sub formulae of Z^{b}):

(ZbI-I)
(SEQ ID NO: 32)
F^{#}-S-S^{#}-W-G;
(ZbII-I)
(SEQ ID NO: 33)
F^{#}-S-S^{#}-W-A;
(ZbIII-I)
(SEQ ID NO: 34)
F^{#}-N-S^{#}-W-A;
(ZbIV-I)
(SEQ ID NO: 35)
F^{#}-Y-S^{#}-W-G;
or
(ZbV-I)
(SEQ ID NO: 36)
F^{#}-N-A^{#}-W-A;

wherein any residue marked with a “^{#}” is independently either the listed unmodified amino acid, a modified amino acid or a non-natural amino acid analogue.

In a particular embodiment, compounds of Formula (Ib) find particular use as insecticides against hemiptera insects (e.g. Halyomorpha halys or Myzus persicae insects) when the Z^{b} is a peptide with a formula selected from (ZbI) or (ZbI-I) (sub formulae of Z^{b}):

(ZbI)
(SEQ ID NO: 24)
F^{#}-S^{#}-S^{#}-W^{#}-G^{#};
or
(ZbI-I)
(SEQ ID NO: 32)
F^{#}-S-S^{#}-W-G;

wherein any residue marked with a “^{#}” is independently either the listed unmodified amino acid, a modified amino acid or a non-natural amino acid analogue.

There is also provided the use as an insect control agent, of a compound according to Formula (Ib) as defined herein. Suitably, said use is as an insecticide against hemiptera insects (e.g. Halyomorpha halys or Myzus persicae insects).

There is also provided a method of increasing hemiptera insect mortality, the method comprising contacting a hemiptera insect (e.g. Halyomorpha halys or Myzus persicae insect) or hemiptera (e.g. Halyomorpha halys or Myzus persicae) insect population with a compound according Formula (Ib) as defined herein, or a composition comprising a compound according to Formula (Ib) as defined herein.

There is also provided a compound according to Formula (Ib) as defined herein, or a composition comprising a compound according to Formula (Ib) as defined herein, as a plant protection agent, for protecting a plant against hemiptera insects (e.g. Halyomorpha halys or Myzus persicae insects).

There is also provided a method of inhibiting infestation of a plant by hemiptera insects (e.g. Halyomorpha halys or Myzus persicae insects), the method comprising contacting the plant with a compound according Formula (Ib) as defined herein, or a composition comprising a compound according to Formula (Ib) as defined herein.

Suitably, the compound is applied to the plant while the plant is free or substantially free of hemiptera insects (e.g. Halyomorpha halys or Myzus persicae insects).

Although the compounds of Formula (Ib), and sub formulae thereof, particular use against insects of the type hemiptera (e.g. Halyomorpha halys or Myzus persicae insects), they may also be applied against any of the insect orders or species described in the methods and uses described herein, e.g. diptera and lepidoptera insects.

Suitably, the compounds of Formula (Ib) may be used in combination with one or more additional insecticides described herein.

In an embodiment, the compounds of Formula (Ib) may be used as an insect control agent against M. persicae, in combination with one or more additional insecticides described herein.

In an embodiment, the compounds of Formula (Ib) may be used as an insect control agent against insects of the type hemiptera in combination with one or more additional insecticides described herein.

The additional insecticide may be selected from a CAPA peptide, e.g. SDSKNT[n-me-A]LWFGPRL-[NH₂](SEQ ID NO: 134) (SB-P-015), ASG[b³L]VAFPRV-[NH₂](SEQ ID NO: 135) (2315), or Palm-LVAFPRV-[NH₂](SEQ ID NO: 136) (AH59).

In a particular embodiment, compounds of Formula (Ib) find particular use as insecticides against blattodea insects (e.g. Blattella Germanica species). Suitably, in such embodiments Z^{b} is a peptide with a formula selected from any one of (ZbI-I), (ZbIII-I), or (ZbIV-I) (sub formulae of Z^{b}):

(ZbI-I)
(SEQ ID NO: 32)
F^{#}-S-S^{#}-W-G;
(ZbIII-I)
(SEQ ID NO: 34)
F^{#}-N-S^{#}-W-A;
or
(ZbIV-I)
(SEQ ID NO: 35)
F^{#}-Y-S^{#}-W-G;

wherein any residue marked with a “^{#}” is independently either the listed unmodified amino acid, a modified amino acid or a non-natural amino acid analogue.

### Compounds of Formula (Ic)

The invention also relates to compounds of Formula (Ic) (a sub formula of Formula (I)) which find particular use against insects of the order lepidoptera (e.g. Plutella xylostella species or Spodeptera frugiperda species).

Therefore, in a particular embodiment of the invention, the invention provides an insecticidal compound having the formula (Ic) below:

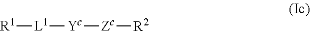

wherein:
   R¹, L¹, and R² are as defined herein;
   Y^{c} is a group Y as defined herein, optionally wherein Y^{c} is absent or is a peptide comprising from 1 to 12 amino acids; and
   Z^{c} is a peptide of the formula:

(SEQ ID NO: 2)
-F^{#}-S^{#}-X^{3c}-W^{#}-G^{#}

wherein X^{3c} is selected from amino acid residues A^{#}, P^{#}, V^{#} or T^{#};
wherein “^{#}” indicates that the listed residue is independently the unmodified amino acid, a modified amino acid or a non-natural amino acid analogue.

Suitably, Z^{c} is a peptide of the formula:

(ZcI)
(SEQ ID NO: 118)
-F-S-X^{3c}-W-G

and X^{3c} is selected from amino acid residues A^{#}, P^{#}, V^{#} or T^{#}, wherein any residue marked with a “^{#}” is independently either the listed unmodified amino acid, a modified amino acid or a non-natural amino acid analogue.

Suitably, X^{3c} is P^{#} or V^{#}.

Suitably, Z^{c} is a peptide of the formula selected from ZcI-I to ZcI-IV:

(ZcI-I)
(SEQ ID NO: 119)
-F-S-A^{#}-W-G
(ZcI-II)
(SEQ ID NO: 120)
-F-S-P^{#}-W-G
(ZcI-III)
(SEQ ID NO: 121)
-F-S-V^{#}-W-G
(ZcI-IV)
(SEQ ID NO: 122)
-F-S-T^{#}-W-G

wherein any residue marked with a “^{#}” is independently either the listed unmodified amino acid, a modified amino acid or a non-natural amino acid analogue.

Suitably, at least one of the residues in the motif Y^{c}—Z^{c} is a modified amino acid or non-natural amino acid analogue.

Suitably, the one or more modified amino acids or a non-natural amino acid analogues in the motif Y^{c}—Z^{c} are independently as defined anywhere herein. The one or more modified amino acids or a non-natural amino acid analogues are independently as defined in any one of paragraphs (11) to (15) herein. More suitably, the one or more modified amino acids or a non-natural amino acid analogues are independently as defined in any one of paragraphs (13), (14) or (15).

In an embodiment of the compounds of formula (Ic), the one or more modified amino acids or a non-natural amino acid analogues is independently selected from an N-methylated amino acid, aminoisobutyric acid (Aib), thiazolidine-4-carboxylic acid (Thz), a beta amino acid or a D-amino acid.

Suitably, Z^{c} is a peptide of the formula selected from:

(SEQ ID NO: 101)
FSPWG
(SEQ ID NO: 102)
FSAWG
(SEQ ID NO: 103)
FSVWG
(SEQ ID NO: 104)
FSTWG
(SEQ ID NO: 105)
FS-βA-WG
(SEQ ID NO: 106)
FS-Thz-WG

Suitably, Y^{c} is absent or a peptide from 1 to 12 amino acids. More suitably, Y^{c} is absent or a peptide from 1 to 8 amino acids. More suitably, Y^{b} is a peptide comprising from 1 to 4 amino acids.

Suitably, Y^{c} is absent or selected from:
VR;
N;
KVK;
SFS; or
Y.

Particular compounds of Formula (Ic) include the compounds in the table below, or salts or solvates thereof:

| Reference Number | Sequence | SEQ ID NO |
|---|---|---|
| SB-P-068 | Hy-VRFSPWG-[NH₂] | SEQ ID NO: 107 |
| SB-P-069 | Hy-NFSPWG-[NH₂] | SEQ ID NO: 108 |
| SB-P-070 | Hy-KVKFSAWG-[NH₂] | SEQ ID NO: 109 |
| SB-P-071 | Hy-SFSPWG-[NH₂] | SEQ ID NO: 110 |
| SB-P-072 | Hy-FSPWG-[NH₂] | SEQ ID NO: 111 |
| SB-P-073 | Hy-YFSPWG-[NH₂] | SEQ ID NO: 112 |
| SB-P-074 | Hy-KVKFSVWG-[NH₂] | SEQ ID NO: 113 |
| SB-P-075 | Hy-KVKFSTWG-[NH₂] | SEQ ID NO: 114 |
| SB-P-076 | Hy-VRFSPWG-[NH₂] | SEQ ID NO: 115 |
| SB-P-102 | Ava-FS-βA-WG-[NH₂] | SEQ ID NO: 116 |
| SB-P-103 | Ava-FS-Thz-WG-[NH₂] | SEQ ID NO: 117 |

### Particular Applications of Compounds of Formula (Ic)

Suitably, compounds of Formula (Ic) find particular use as insecticides against lepidoptera insects (e.g. Plutella xylostella or Spodoptera frugiperda). Such compounds may therefore be applied in the appropriate methods defined herein.

There is also provided the use as an insect control agent, of a compound according to Formula (Ic) as defined herein. Suitably, said use is as an insecticide against lepidoptera insects (e.g. Plutella xylostella or Spodoptera frugiperda).

There is also provided a method of increasing lepidoptera insect mortality, the method comprising contacting a lepidoptera insect (e.g. Plutella xylostella or Spodoptera frugiperda) or lepidoptera insect population (e.g. a Plutella xylostella population) with a compound according Formula (Ic) as defined herein, or a composition comprising a compound according to Formula (Ic) as defined herein.

There is also provided a compound according to Formula (Ic) as defined herein, or a composition comprising a compound according to Formula (Ic) as defined herein, as a plant protection agent, for protecting a plant against lepidoptera insects (e.g. Plutella xylostella or Spodoptera frugiperda).

There is also provided a method of inhibiting infestation of a plant by lepidoptera insects (e.g. Plutella xylostella or Spodoptera frugiperda), the method comprising contacting the plant with a compound according Formula (Ic) as defined herein, or a composition comprising a compound according to Formula (Ic) as defined herein. Suitably, the compound is applied to the plant while the plant is free or substantially free of lepidoptera insects (e.g. Plutella xylostella or Spodoptera frugiperda).

The compounds of Formula (Ic) may find particular use as insecticides against Plutella xylostella species.

The compounds of Formula (Ic) may find particular use as insecticides against Spodeptera frugiperda species.

In a particular embodiment, compounds of Formula (Ic) find particular use as insecticides against lepidoptera insects (e.g. Plutella xylostella or Spodoptera frugiperda), when the Z^{c} moiety is a peptide of the formula selected from any one of ZcI-I to ZcI-IV:

(ZcI-I)
(SEQ ID NO: 119)
-F-S-A^{#}-W-G
(ZcI-II)
(SEQ ID NO: 120)
-F-S-P^{#}-W-G
(ZcI-III)
(SEQ ID NO: 121)
-F-S-V^{#}-W-G
(ZcI-IV)
(SEQ ID NO: 122)
-F-S-T^{#}-W-G

wherein any residue marked with a “^{#}” is independently either the listed unmodified amino acid, a modified amino acid or a non-natural amino acid analogue.

In a particular embodiment, compounds of Formula (Ic) find particular use as insecticides against lepidoptera insects (e.g. Plutella xylostella or Spodoptera frugiperda), when the Z^{c} moiety is a peptide of the formula selected from any one of ZcI-I, ZcI-II or ZcI-IV:

(ZcI-I)
(SEQ ID NO: 119)
-F-S-A^{#}-W-G
(ZcI-II)
(SEQ ID NO: 120)
-F-S-P^{#}-W-G
(ZcI-IV)
(SEQ ID NO: 122)
-F-S-T^{#}-W-G

wherein any residue marked with a “^{#}” is independently either the listed unmodified amino acid, a modified amino acid or a non-natural amino acid analogue.

Although the compounds of Formula (Ic), and sub formulae thereof, particular use against insects of the type lepidoptera (e.g. Plutella xylostella or Spodoptera frugiperda), they may also be applied against any of the insect orders and species described in the methods and uses described herein, e.g. diptera and hemiptera insects.

Suitably, the compounds of Formula (Ic) may be used in combination with one or more additional insecticides described herein.

In an embodiment, the compounds of Formula (Ic) may be used as an insect control agent against insects of the type lepidoptera in combination with one or more additional insecticides described herein.

In one embodiment, the compound of the invention may be any of the kinin analogues listed herein. Examples of kinin analogue peptides include the compounds disclosed herein, and salts or solvates thereof, in particular those in the following table:

| Reference | | |
|---|---|---|
| Number | Sequence | SEQ ID NO |
| SB-P-003 | Hy-DPKYKFSSWG-[NH₂] | SEQ ID NO: 59 |
| SB-P-004 | Hy-AKFSSWG-[NH₂] | SEQ ID NO: 60 |
| SB-P-018 | Hy-ARFSSWA-[NH₂] | SEQ ID NO: 61 |
| SB-P-019 | Hy-SKFNSWA-[NH₂] | SEQ ID NO: 62 |
| SB-P-020 | Hy-AKFSSWA-[NH₂] | SEQ ID NO: 63 |
| SB-P-021 | Hy-ARFNSWA-[NH₂] | SEQ ID NO: 64 |
| SB-P-022 | Hy-VPFNSWA-[NH₂] | SEQ ID NO: 65 |
| SB-P-023 | Hy-PIFSSWG-[NH₂] | SEQ ID NO: 66 |
| SB-P-024 | Hy-GPDFYAWG-[NH₂] | SEQ ID NO: 67 |
| SB-P-032 | Ac-KQrFH[Aib]WG-[NH₂] | SEQ ID NO: 18 |
| SB-P-033 | Ac-NSVVLGKKQrFH[Aib]WG-[NH₂] | SEQ ID NO: 19 |
| SB-P-034 | Ac-FH[Aib]WG-[NH₂] | SEQ ID NO: 20 |
| SB-P-035 | Ac-KQrFHsWG-[NH₂] | SEQ ID NO: 21 |
| SB-P-036 | Ac-FHsWG-[NH₂] | SEQ ID NO: 22 |
| SB-P-052 | Hy-NSVVLGKKQ[n-me-R]FH[n-me-S]WG- | SEQ ID NO: 23 |
| | [NH₂] | |
| SB-P-054 | Ac-SkFN-Aib-WA-[NH₂] | SEQ ID NO: 68 |
| SB-P-056 | Ac-SK-[n-me-F]-N-(α-Me)Ser-WA-[NH₂] | SEQ ID NO: 69 |
| SB-P-060 | Ac-PA-[n-me-F]-SsWG-[NH₂] | SEQ ID NO: 70 |
| SB-P-065 | Hy-RQKTVFSSWG-[NH₂] | SEQ ID NO: 71 |
| SB-P-066 | Hy-PAFSSWG-[NH₂] | SEQ ID NO: 72 |
| SB-P-067 | Hy-ASDKHGRPKQTFSSWG-[NH₂] | SEQ ID NO: 73 |
| SB-P-068 | Hy-VRFSPWG-[NH₂] | SEQ ID NO: 107 |
| SB-P-069 | Hy-NFSPWG-[NH₂] | SEQ ID NO: 108 |
| SB-P-070 | Hy-KVKFSAWG-[NH₂] | SEQ ID NO: 109 |
| SB-P-071 | Hy-SFSPWG-[NH₂] | SEQ ID NO: 110 |
| SB-P-072 | Hy-FSPWG-[NH₂] | SEQ ID NO: 111 |
| SB-P-073 | Hy-YFSPWG-[NH₂] | SEQ ID NO: 112 |
| SB-P-074 | Hy-KVKFSVWG-[NH₂] | SEQ ID NO: 113 |
| SB-P-075 | Hy-KVKFSTWG-[NH₂] | SEQ ID NO: 114 |
| SB-P-076 | Hy-VRFSPWG-[NH₂] | SEQ ID NO: 115 |
| SB-P-100 | Ac-PA-[n-me-F]-S-βA-WG-[NH₂] | SEQ ID NO: 74 |
| SB-P-101 | Ac-PA-[n-me-F]-S-(α-Me)S-WG-[NH₂] | SEQ ID NO: 75 |
| SB-P-102 | Ava-FS-BA-WG-[NH₂] | SEQ ID NO: 116 |
| SB-P-103 | Ava-FS-Thz-WG-[NH₂] | SEQ ID NO: 117 |

Further Examples of kinin analogue peptides include the compounds disclosed herein, and salts or solvates thereof, in particular those in the following table:

| Reference | | |
|---|---|---|
| Number | Sequence | SEQ ID NO |
| SB-P-003 | Hy-DPKYKFSSWG-[NH₂] | SEQ ID NO: 59 |
| SB-P-004 | Hy-AKFSSWG-[NH₂] | SEQ ID NO: 60 |
| SB-P-018 | Hy-ARFSSWA-[NH₂] | SEQ ID NO: 61 |
| SB-P-019 | Hy-SKFNSWA-[NH₂] | SEQ ID NO: 62 |
| SB-P-020 | Hy-AKFSSWA-[NH₂] | SEQ ID NO: 63 |
| SB-P-021 | Hy-ARFNSWA-[NH₂] | SEQ ID NO: 64 |
| SB-P-022 | Hy-VPFNSWA-[NH₂] | SEQ ID NO: 65 |
| SB-P-023 | Hy-PIFSSWG-[NH₂] | SEQ ID NO: 66 |
| SB-P-024 | Hy-GPDFYAWG-[NH₂] | SEQ ID NO: 67 |
| SB-P-032 | Ac-KQrFH[Aib]WG-[NH₂] | SEQ ID NO: 18 |
| SB-P-033 | Ac-NSVVLGKKQrFH[Aib]WG-[NH₂] | SEQ ID NO: 19 |
| SB-P-034 | Ac-FH[Aib]WG-[NH₂] | SEQ ID NO: 20 |
| SB-P-035 | Ac-KQrFHsWG-[NH₂] | SEQ ID NO: 21 |
| SB-P-036 | Ac-FHSWG-[NH₂] | SEQ ID NO: 22 |
| SB-P-052 | Hy-NSVVLGKKQ[n-me-R]FH[n-me-S]WG- | SEQ ID NO: 23 |
| | [NH₂] | |
| SB-P-054 | Ac-SKFN-Aib-WA-[NH₂] | SEQ ID NO: 68 |
| SB-P-056 | Ac-SK-[n-me-F]-N-(α-Me)Ser-WA-[NH₂] | SEQ ID NO: 69 |
| SB-P-060 | Ac-PA-[n-me-F]-SsWG-[NH₂] | SEQ ID NO: 70 |
| SB-P-065 | Hy-RQKTVFSSWG-[NH₂] | SEQ ID NO: 71 |
| SB-P-066 | Hy-PAFSSWG-[NH₂] | SEQ ID NO: 72 |
| SB-P-067 | Hy-ASDKHGRPKQTFSSWG-[NH₂] | SEQ ID NO: 73 |
| SB-P-068 | Hy-VRFSPWG-[NH₂] | SEQ ID NO: 107 |
| SB-P-069 | Hy-NFSPWG-[NH₂] | SEQ ID NO: 108 |
| SB-P-070 | Hy-KVKFSAWG-[NH₂] | SEQ ID NO: 109 |
| SB-P-071 | Hy-SFSPWG-[NH₂] | SEQ ID NO: 110 |
| SB-P-072 | Hy-FSPWG-[NH₂] | SEQ ID NO: 111 |
| SB-P-073 | Hy-YFSPWG-[NH₂] | SEQ ID NO: 112 |
| SB-P-074 | Hy-KVKFSVWG-[NH₂] | SEQ ID NO: 113 |
| SB-P-075 | Hy-KVKFSTWG-[NH₂] | SEQ ID NO: 114 |
| SB-P-076 | Hy-VRFSPWG-[NH₂] | SEQ ID NO: 115 |
| SB-P-100 | Ac-PA-[n-me-F]-S-βA-WG-[NH₂] | SEQ ID NO: 74 |
| SB-P-101 | Ac-PA-[n-me-F]-S-(α-Me)S-WG-[NH₂] | SEQ ID NO: 75 |
| SB-P-102 | Ava-FS-BA-WG-[NH₂] | SEQ ID NO: 116 |
| SB-P-103 | Ava-FS-Thz-WG-[NH₂] | SEQ ID NO: 117 |
| SB-P-143 | Hy-DPAFNSWG-[NH₂] | SEQ ID NO: 76 |
| SB-P-144 | Hy-SSAFNSWG-[NH₂] | SEQ ID NO: 77 |
| SB-P-145 | Hy-RAFTSTGSSRSPAFSSWG-[NH₂] | SEQ ID NO: 78 |
| SB-P-146 | Hy-PASFSSWG-[NH₂] | SEQ ID NO: 79 |
| SB-P-147 | Hy-DAGFSSWG-[NH₂] | SEQ ID NO: 80 |
| SB-P-148 | Hy-SSGFSSWG-[NH₂] | SEQ ID NO: 81 |
| SB-P-149 | Hy-DPVFKSWG-[NH₂] | SEQ ID NO: 82 |
| SB-P-150 | Hy-DAAFSSWG-[NH₂] | SEQ ID NO: 83 |
| SB-P-151 | Hy-GSGFSSWG-[NH₂] | SEQ ID NO: 84 |
| SB-P-154 | Hy-DDDSDVSESGSKFSSWG-[NH₂] | SEQ ID NO: 85 |
| SB-P-155 | Hy-SFSSWG-[NH₂] | SEQ ID NO: 86 |
| SB-P-156 | Hy-GFSSWG-[NH₂] | SEQ ID NO: 87 |
| SB-P-157 | Hy-LDRSEGRVSKALFSSWG-[NH₂] | SEQ ID NO: 88 |
| SB-P-158 | Hy-WGQSSVGAVFSSWG-[NH₂] | SEQ ID NO: 89 |
| SB-P-159 | Hy-GAEFYSWG-[NH₂] | SEQ ID NO: 90 |
| SB-P-169 | Hy-FSSWG-[NH₂] | SEQ ID NO: 91 |
| N/A | Hy-GPAFSSWG-[NH₂] | SEQ ID NO: 92 |
| N/A | Hy-AAAFNSWG-[NH₂] | SEQ ID NO: 93 |
| N/A | Hy-NAAFSSWG-[nh2] | SEQ ID NO: 94 |
| N/A | Hy-NAAFNSWG-[NH₂] | SEQ ID NO: 95 |
| N/A | Hy-VPAFNSWG-[NH₂] | SEQ ID NO: 96 |
| N/A | Hy-TNTAFSSWG-[NH₂] | SEQ ID NO: 97 |
| N/A | Hy-PRFYSWG-[NH₂] | SEQ ID NO: 98 |
| N/A | Hy-GADFYAWG-[NH₂] | SEQ ID NO: 99 |
| N/A | [Ac]-SK[n-me-F]N[βAla]WA-[NH₂] | SEQ ID NO: 100 |

### Further Explanation of Compounds of the Invention

In some embodiments, the compounds of the invention may be in the form of a salt or solvate.

The compounds of the invention may be provided in combination with one or more additional active insecticides, such as those described herein.

R¹, L¹ and R²

The terminal groups present at the N- and C-termini of the peptide backbone are designated R¹ and R² respectively. Thus R¹ is bonded (optionally via L¹ or Y¹) to the nitrogen atom of the N-terminal amino group of Y and R² is bonded to the C-terminal carbonyl carbon atom of Z or Y².

In addition to comprising at least one modified or unnatural amino acid residue, the compounds of the invention may include further functionalisation, suitably at the N or C terminus. Suitably, the compounds may be functionalised to increase cuticle permeability or to increase stability. Suitably, the compound may be functionalised with an aromatic, aliphatic or lipophilic group. Suitably therefore, R¹ may be an aromatic, heteroaromatic, aliphatic or lipophilic group.

In certain embodiments, the compound may be functionalised with a lipophilic group such as a fatty acyl group. Fatty acyl groups include but are not limited to palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl. Suitably therefore the R¹ group may be palmitoyl ([Palm]).

In one embodiment, the compound may be functionalised with an aromatic group such as a benzyl or benzoyl group which may be a benzoic acid derivative or benzophenone derivative. Suitably therefore the R¹ group may be an aromatic group such as 4-benzoyl benzoic acid, or a derivative such as 4-benzoyl benzoyl.

In one embodiment, the compound may be functionalised with an acyl group.

An “acyl” group is a group of the formula R^{3a}—C(O)— group wherein R^{3a} is a C₁₋₆alkyl, for example formyl, acetyl (Ac), propanoyl, butanoyl, or wherein R^{3a} is benzoyl. Suitably, the R^{3a} group may be R^{1b}—C(O)—, such as acetyl (Ac).

In some embodiments, the R¹ group may be a biotin moiety. Thus, a biotin moiety may be incorporated into an amino acid residue (e.g. in a modified lysine side chain) or at the terminus.

In certain embodiments, the R¹ group may be substituted with a sugar moiety. An amino acid residue may also be modified with a sugar moiety, i.e. the amino acid residue may be a “sugar modified analogue”. The sugar moiety may be a monosaccharide or disaccharide. Examples of monosaccharides include glucose, 6-deoxyglucose, mannose, galactose, glucosamine, galactosamine, N-acetylglucosamine, N-acetylgalactosamine, glucuronic acid, allose, altrose, gulose, idose, fucose, talose, ribose, deoxyribose, arabinose, xylose, lyxose, ribulose, xylulose, fructose, psicose, sorbose or tagatose. Examples of disaccharides include sucrose, lactose, lactulose, allolactose, maltose, isomaltose, isomaltulose, trehalose, cellobiose, kojibiose, nigerose, sophorose, laminaribiose, gentiobiose, thiomaltose, mannobiose or their N-, C- or S-interglycosidic derivative. Most suitably, the sugar moiety is selected from glucosamine or galactosamine.

The sugar can be N-terminus modification or as part of Ser sidechain modification. As part of N-terminus, a spacer is usually added (e.g. succinic acid, Suc). An example of a R¹-L¹-Z sequence with such a modification is: sugar-Suc-SDSKNTAL. The notation “sugar” can be any of the sugars described herein, for example glucosamine or galactosamine.

Suitably the compound may also be modified with a (poly)alkyleneglycol polymer. This modification may be present in the R¹, L¹ or Z moiety. Suitably, the R¹ group may be a (poly)alkyleneglycol. The presence of the polymer may increase the ease of formulation of the compound. Preferred (poly)alkyleneglycols include polyethylene glycol (PEG). The compound may therefore be PEGylated, suitably by covalent attachment of polyethylene glycol to an amino acid residue in Z, at the R¹ position, or via a linker L¹. The PEG group may have any suitable terminal group, such as NH₂, OH or Ome.

PEG groups present as a linker portion or as part of a modified peptide chain have the formula [—(OCH₂CH₂)ₙ—], where n may preferably be an integer from 1 to 16.

R¹ is selected from hydrogen (which may be designated “H-” or “Hy-”, or may not be noted in the specific peptide sequence), C₁₋₄ alkyl (e.g. methyl, ethyl, propyl, butyl), —N(R^{1a})—C(═N⁺(R^{1b})(R^{1c}))NR^{1d}R^{1e}, or —C(═N⁺(R^{1b})(R^{1c}))NR^{1d}R^{1e}; wherein each of R^{1a}, R^{1b}, R^{1c}, R^{1d} and R^{1e} is independently selected from hydrogen or C₁₋₄ alkyl (e.g. methyl, ethyl, propyl, butyl), preferably hydrogen or methyl.

In some embodiments, if R¹ is —C(═N⁺(R^{1b})(R^{1c}))NR^{1d}R^{1e}; each of R^{1a}, R^{1b}, R^{1c}, R^{1d} and R^{1e} are methyl, i.e. R¹ is —C(═N⁺Me₂)Nme₂.

When R¹═“H” (or “Hy”;), it typically indicates a free primary amino group at the N-terminus. The other hydrogen atom of the N-terminal amino group is typically invariant, regardless of the nature of R¹. Exceptionally, when the residue at the N-terminus is N-methylated, R¹ may still be indicated as H even though the N-terminal residue has a secondary amine group. Thus, an N-methylated leucine residue at the N-terminus may be indicated as R¹-[n-me-L]- where R¹ is H. However, it could also be shown as simply R¹-L- where R¹ is methyl and the other hydrogen atom is not shown.

In certain embodiments, L¹ may comprise a carbonyl group C═O adjacent to the N-terminus of the Y¹, Y or Z group. For example, L¹ may be *—(C═O)C₁₋₁₂-alkylene-NH—, —(C═O)—C₁₋₁₀alkylene-(C═O)—, *—(C═O)—C₁₋₁₀alkylene, (e.g. —(C═O)—, i.e. C₁alkylene optionally substituent by oxo); wherein any L¹ group is optionally substituted with one or more oxo (═O) groups; where * denotes the point of attachment to Y¹, Y or Z

In some embodiments when R¹ is hydrogen, L¹ is absent or *—(C═O)C₁₋₁₆-alkylene-NH— where * denotes the point of attachment to Y¹, Y or Z, e.g. R¹ is hydrogen and L¹ is *—(C═O)C₁₋₆-alkylene-NH— such as:

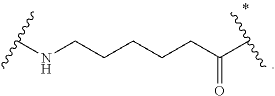 [SMILES: *NCCCCCC(*)=O]

In certain embodiments, L¹, if present, is *—(C═O)C₁₋₁₀-alkylene-NH— where * denotes the point of attachment to Y¹, Y or Z. For example, L¹ may be *—(C═O)C₁₋₆-alkylene-NH—, such as:

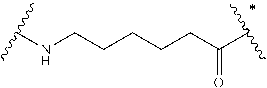 [SMILES: *NCCCCCC(*)=O]

which may be regarded as a residue of 6-aminohexanoic acid (Ahx).

In some embodiments when R¹ is hydrogen, L¹ is absent or *—(C═O)C₁₋₁₆-alkylene-NH— where * denotes the point of attachment to Y¹, Y or Z, e.g. R¹ is hydrogen and L¹ is *—(C═O)C₁₁alkylene-NH₂, where * denotes the point of attachment to Y¹, Y or Z, i.e.

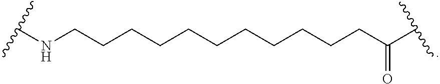 [SMILES: *NCCCCCCCCCCCC(*)=O]

In certain embodiments, L¹, if present, is *—(C═O)C₁₋₁₂-alkylene-NH— where * denotes the point of attachment to Y¹, Y or Z. For example, L¹ may be *—(C═O)C₆₋₁₂-alkylene-NH—, such as *—(C═O)C₁₁alkylene-NH₂, where * denotes the point of attachment to Y¹, Y or Z, i.e.

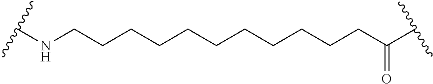 [SMILES: *NCCCCCCCCCCCC(*)=O]

which may be regarded as a residue of 12-aminododecanoic acid (Ado).

When L¹ is present, R¹ is typically hydrogen (H). For example, R¹ is hydrogen and L¹ is *—(C═O)C₁₋₆-alkylene-NH— such as:

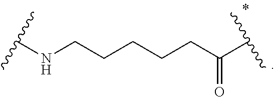 [SMILES: *NCCCCCC(*)=O]

In some embodiments, when R¹ is —C(═N⁺(R^{1b})(R^{1c}))NR^{1d}R^{1e}, L¹ is absent e.g. R¹ is —C(═N⁺Me₂)NMe₂ and L¹ is absent. In such embodiments the R¹ group forms the following guanidine based structure along with the N-terminal nitrogen (denoted “N—R” in the structure below):

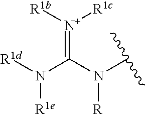

Typically, when L¹ is —(C═O)C₁₋₁₀-alkylene-NH—, R¹ is H.

Typically when R¹ is —C(═N⁺(R^{1b})(R^{1c}))NR^{1d}R^{1e}, L¹ is absent. When R¹ is —C(═N⁺(R^{1b})(R^{1c}))NR^{1d}R^{1e} and L¹ is absent, the R¹ group together with the N-terminal nitrogen of the peptide sequence Z form a guanidine based group (discussed above). Preferably “—C(═N⁺(R^{1b})(R^{1c}))NR^{1d}R^{1e}” is-C(═N⁺Me₂)NMe₂.

R² is NH₂, NR^{2a}H, NR^{2a}R^{2b}, or OR^{2a}; wherein each of R^{2a} and R^{2b} are as defined herein. Preferably R² is NH₂ (denoted in sequences as [NH₂])

“Guanidyl” refers to the case where the R¹ is —C(═N⁺Me₂)NMe₂ and the terminal structure formed by “Guanidyl-L-” is as follows:

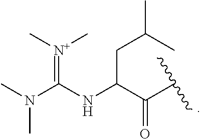 [SMILES: *C(=O)C(CC(C)C)NC(N(C)C)=[N+](C)C]

Y¹ and Y²

The compound as defined herein may comprise one or two further peptide groups located between the terminal R¹ and R² groups at the N and C terminus respectively.

Suitably these groups are the Y¹ and Y² group. If present, these groups are located on either side of the Z peptide group.

In one embodiment, the Y¹ and Y² groups are absent.

In one embodiment, the compound comprises a Y¹ group only. In one embodiment, the compound comprises a Y² group only.

In one embodiment, the compound comprises a Y¹ and Y² group.

Suitably the Y¹ and Y² groups when present independently comprise one or two amino acids. Suitably the Y¹ and Y² groups may comprise any amino acid, suitably independently selected from: M, R, G, and K.

In one embodiment, the Y¹ and Y² groups are independently selected from: GR, RG, KR, RK, K, R, M, and G. In one embodiment, the Y¹ and Y² groups are independently selected from: RG, KR, and K.

In one embodiment, the compound comprises a Y² group only, wherein the Y² group is selected from RG, KR, and K.

### Modified Amino Acid or Non-Natural Amino Acid Analogue

In certain embodiments, at least one of the residues in peptides Y and Z (including sub-formulae thereof) is a modified or non-natural amino acid analogue. The modified or non-natural amino acid analogue may be any analogue known to a skilled person. Examples of modified or non-natural amino acid analogues include:
i. N-methylated amino acid;
ii. a peptoid analogue;
iii. a sugar modified analogue;
iv. a biotin modified analogue
v. beta amino acids,
vi. D-configuration amino acid,
vii. non-proteinogenic amino acids, such as hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib), thienyl alanine (Thi), thiazolidine-4-carboxylic acid (Thz), phenylglycine (Phg) or gamma aminobutyric acid (gaba), etc.
viii α-alkylated amino acids;
   or the side chains of two amino acid residues may be joined together to form a cyclic structure (e.g. intramolecular amide formation via condensation reaction between the side chains of arginine or lysine, with the side chain of aspartic acid or glutamic acid.

The compounds of the invention may comprise one or more modified amino acid or non-natural amino acid analogues in the Y peptide, the Z peptide or both the Y and Z peptides.

In certain embodiments, at least one of the residues in the Y moiety is a modified or non-natural amino acid analogue.

The compounds of the invention may comprise multiple modified amino acid or non-natural amino acid analogues in the peptide sequence Y—Z.

In an embodiment, the peptide moiety Y—Z (including sub-formulae thereof) comprises five unmodified amino acid residues.

In an embodiment, the peptide moiety Y—Z (including sub-formulae thereof) comprises four unmodified amino acid residues, and one modified amino acid or non-natural amino acid analogues.

In an embodiment the peptide moiety Y—Z (including sub-formulae thereof) comprises three unmodified amino acid residues, and two modified amino acid or non-natural amino acid analogues.

In an embodiment, the peptide moiety Y—Z (including sub-formulae thereof) comprises two unmodified amino acid residues, and four modified amino acid or non-natural amino acid analogues.

Suitably, at least two of the residues in the peptide moiety Y—Z (including sub-formulae thereof) are the unmodified amino acid. More suitably, at least three of the residues in the peptide moiety Y—Z are the unmodified amino acid.

In certain embodiments, the peptide Y—Z (including sub-formulae thereof) comprises 1, 2, 3 or 4 modified amino acids or non-natural amino acid analogues. In certain embodiments, the peptide Y—Z comprises 1, 2 or 3 modified amino acids or non-natural amino acid analogues. In certain embodiments, the peptide Y—Z comprises 1 or 2 modified amino acids or non-natural amino acid analogues. In certain embodiments, the peptide Y—Z comprises a single modified amino acid or non-natural amino acid analogue.

In certain embodiments, from 1 to 5 of the residues in peptide moiety Y—Z (including sub-formulae thereof) are a modified amino acid or non-natural amino acid analogue. In certain embodiments, from 1 to 4 of the residues in peptide moiety Y—Z are a modified amino acid or non-natural amino acid analogue. In certain embodiments, from 1 to 3 of the residues in peptide moiety Y—Z are a modified amino acid or non-natural amino acid analogue.

In certain embodiments, the peptide Y—Z (including sub-formulae thereof) comprises no more than 5 modified amino acids or non-natural amino acid analogues. In certain embodiments, the peptide Y—Z comprises no more than 4 modified amino acids or non-natural amino acid analogues. In certain embodiments, the peptide Y—Z comprises no more than 3 modified amino acids or non-natural amino acid analogues.

In other embodiments, the peptide Y—Z (including sub-formulae thereof) does not comprise any a modified amino acid or non-natural amino acid analogues. Thus, the peptide Y—Z (including sub-formulae thereof) may comprise only unmodified amino acids, i.e. each residue is the listed proteinogenic amino acid residue.

### Further Discussion of the Invention

### Activity

Suitably, the compounds have activity against hemipteran insects and/or dipteran insects. Preferably, the compounds have activity against hemipteran insects. The compounds of the invention may therefore find particular use against hemipteran insects.

Suitably, the compound have activity against blattodea and/or lepidoptera insects.

The compounds typically increase insect mortality, for example when contacted topically to a suitable insect, or ingested by a suitable insect. Thus, the compounds described (and compositions containing them) may be regarded as insecticides, and may be referred to as “insect control agents”.

Without wishing to be bound by theory, any or all of the effects described may be mediated by agonist activity at the kinin receptor of the target insects. The kinin receptors believed to be present in target insects include the GPCRs kinin. Suitably the compounds of the invention bind to one or more kinin receptors of target insects. Suitably the compounds of the invention have agonist activity when bound to a kinin receptor of a target insect. Suitably the compounds of the invention bind to kinin receptor. Suitably the compounds of the invention are agonists of kinin receptor.

It is believed that, inter alia, the peptide analogues described in this specification retain agonist activity while having superior stability compared to wild type kinin peptides, especially against proteases. Consequently, they are believed to have superior applicability as insecticides.

### Insect Control Agent

The term “insect control agent” refers to agents used to increase insect mortality (i.e. as insecticides). Any compound or composition thereof of the invention maybe considered as an insect control agent, and may be used as an insect control agent.

Suitably therefore the invention provides the use of a compound or composition of the invention as an insect control agent, for example against diptera insects, hemiptera insects, blattodea insects and/or lepidoptera insects. Thus, an insect control agent may be administered to accelerate mortality of a given insect or insect population.

An increase in mortality used herein is intended to refer to an increase in the percentage of dead insects, as compared to the percentage of dead insects of an otherwise identical insect population which have not been exposed to the insect control agent of the invention.

Suitably, insect mortality may be calculated as number of dead insects/total number of insects per treated area. Suitably the treated area may be a well of a plate, or may be one or more leaves, or an entire plant. Suitably insect mortality may be measured by performing a leaf dip assay as described herein in the examples.

An insect control agent may be used to reduce the size of an insect population, or inhibit growth of an insect population or inhibit feeding of an insect population (e.g. as compared to an otherwise identical insect population not exposed to the agent).

An insect control composition is a composition comprising an insect control agent as described.

### Plant Protection Agent

The term “plant protection agent” refers to agents used to protect a plant or plant part against insects, e.g. against infestation or colonisation, or being used as a food source by such insects (e.g. by the draining of sap). Any compound or composition thereof of the invention maybe considered as a plant protection agent, and may be used as a plant protection agent. Suitably therefore the invention provides the use of a compound or composition of the invention as a plant protection agent, for example to protect a plant against diptera insects, hemiptera insects, blattodea insects and/or lepidoptera insects.

Infestation or colonisation may be by larvae (or nymphs), by adult insects, or may be by being used as a host or repository for eggs. The terms “infestation” and “colonisation” should not be construed as requiring the presence of the insects to be deleterious to the plant, however.

A plant protection agent may be applied inter alia for reducing insect load on a plant or plant part, for inhibiting or reducing infestation of a plant by insects, for inhibiting (e.g. reducing the rate of) an increase in insect load on a plant or plant part, or for maintaining a plant in an insect-free state, as compared to an otherwise identical plant having an insect population not exposed to the agent. Thus, the plant protection agent may be applied to a plant or plant part which already carries hemipteran, dipteran and/or lepidopteran insects, or to a plant or plant part which is free or substantially free of hemipteran, dipteran, blattodea and/or lepidopteran insects.

A plant protection composition is a composition comprising a plant protection agent i.e. a compound of the invention as described.

### Plants

By ‘plant or plant part’, or ‘plant or part thereof’ referred to herein it is meant any part of a plant including but not limited to; the leaf, stem, root, flower, bud, bulb, and seed.

Suitable plants or parts thereof which may be protected by the compounds or compositions thereof of the invention, or by the agents of the present invention include crops and plants of agricultural, horticultural, or economic significance. Suitable plants may include any of the following or parts thereof:
Musa textilis, Medicago sativa, Prunus dulcis, Pimpinella anisum, Malus sylvestris, Prunus armeniaca, Areca catechu, Arracacia xanthorhiza, Maranta arundinacea, Cynara scolymus, Helianthus tuberosus, Asparagus officinalis, Persea americona, Pennisetum americanum, Vigna subterranean, Musa paradisiaca, Hordeum vulgare, Phaseolus vulgaris, Phaseolus vigna spp., Beta vulgaris, Citrus bergamia, Rubus spp., Piper nigrum, Acacia mearnsii, Vaccinium spp., Bertholletia excelsa, Artocarpus altilis, Vicia faba, Brassica oleracea botrytis, Sorghum bicolor, Brassica oleracea gemmifera, Fagopyrum esculentum, Brassica oleracea capitate, Brassica rapa, Brassica spp., Theobroma cacao, Cucumis melo, Carum carvi, Elettaria cardamomum, Cynara cardunculus, Ceratonia siliqua, Daucus carota, Anacardium occidentale, Manihot esculenta, Ricinus communis, Brassica oleracea botrytis, Apium graveolens, Sechium edule, Prunus spp., Castanea sativa, Cicer arietinum, Cichorium intybus, Cichorium intybus, Capsicum spp., Cinnamomum verum, Cymbopogon nardus, Citrus medica, Citrus veticulata, Trifolium spp., Syzygium aromaticum, Cocos nucifera, Colocasia spp.; Xanthosoma spp., Coffee spp., Cola spp., Brassica napus, Zea mays, Valerianella locusta, Gossypium spp., Vigna unguiculate, Vaccinium spp., Lepidium sativum, Cucumis sativus, Ribes spp., Annona reticulata, Colocasia esculenta, Phoenix dactylifera, Moringa oleifera, Phaseolus spp., Allium sativum, Allium cepa, Pisum sativum, Triticum durum, Xanthosoma spp.; Colocasia spp., Solanum melongena, Cichorium endivia, Lygeum spartum, Foeniculum vulgare, Trigonella foenumgraecum, Ficus carica, Corylus avellane, Furcraea macrophylla, Linum usitatissimum, Phormium tenax, Pelargonium spp.; Geranium spp., Zingiber officinalis, Langenaria spp; Cucurbita spp., Cicer arietinum, Citrus paradise, Vitis vinifera, Lygeum spartum, Dactylis glomerata, Arachis hypogaea, Psidium guajava, Corylus avellane, Cannabis sativa, Crotalaria juncea, Agave fourcroydes, Lawsonia inermis, Humulus lupulus, Armoracia Rusticana, Indigofera tinctorial, Jasminum spp., Corchorus spp., Brassica oleracea acephala, Ceiba pentandra, Hibiscus cannabinus, Brassica oleracea gongylodes, Lavandula spp., Allium ampeloprasum, Citrus limon, Cymbopogon citratus, Lens culinaris, Lespendeza spp., Lactuca sativa, Glycyrrhiza glabra, Citrus aurantifolia, Citrus limetta, Linum usitatissimum, Litchi chinensis, Eriobotrya japonica, Lupinus spp., Macadamia spp., Myristica fragrans, Agave atrovirens, Citrus reticulata, Mangifera indica, Manihot esculenta, Secale cereal, Mespilus germanica, Cucumis melo, Penicum miliaceum, Eleusine coracana, Setaria italica, Echinochloa crusgalli, Eleusine coracana; Mentha spp., Morus spp., Morus alba, Agaricus spp.; Pleurotus spp. Volvariella, Brassica nigra; Sinapis alba, Prunus persica, Phormium tenax, Guizotia abyssinica, Myristica fragrans, Avena spp., Elaeis guineensis, Abelmoschus esculentus, Olea europea, Papaver somniferum, Citrus sinensis, Citrus aurantium, Dactylis glomerate, Metroxylon spp., Borassus flabellifer, Carica papaya, Pastinaca sativa, Pyrus communis, Pisum sativum, Carya illinoensis, Capsicum annuum, Diospyros kaki; Diospyros virginiana, Cajanus cajan, Ananas comosus, Pistacia spp., Prunus domestica, Punica granatum, Citrus grandis, Solamum tuberosum, Ipomoea batatas, Cucurbita spp., Chrysanthemum cineraraiefolium, Aspidosperma spp., Cydonia oblonga, Cinchona spp., Chenopodium quinoa, Raphanus sativus (including Cochlearia armoracia), Boehmeria nivea, Agrostis spp., Boehmeria nivea, Rheum spp., Oryza sativa; Oryza glaberrima, Rose spp., Hevea brasiliensis, Secale cereal, Lolium spp., Carthamus tinctorius, Metroxylon spp., Onobrychis viciifolia, Valerianella locusta, Tragopogon porrifolius, Achras sapota, Citrus reticulata, Brassica ileracea capitate, Scorzonera hispanica, Sesamum indicum, Butyrospermum paradoxum, Agave sislana, Citrus aurantifolia, Glycine max, Triticum spelta, Spinacia oleracea, Secale cereal, Cucurbita spp., Fragaria spp., Sorghum bicolor Sudanense, Saccharum officinarum, Helianthus annuus, Crotalaria juncea, Citrus limetta, lopmoea batatas, Citrus reticulata, Xanthosoma sagittifolium, Manihot esculenta, Colocasia esculenta, Camellia sinensis, Eragrostis abyssinica, Phleum pratense, Nicotiana tabacum, Lycopersicum esculentum, Lotus spp., Aleurites spp., Brassica rapa, Urena lobate, Vanilla planifolia, Vicia sativa, Juglans spp., Citrullus lanatus, Acacia mearnsii, Triticum spp., Hordeum spp., Dioscorea spp., and Ilex paraguariensis.

Suitably, the plant or part thereof which may be protected by the agents of the present invention is selected from a plant which suffers from hemipteran, dipteran and/or lepidopteran insect infestations, or which attracts hemipteran, dipteran and/or lepidopteran insects. Suitably, the plant or part thereof which suffers from hemipteran, dipteran and/or lepidopteran insect infestations, or which attracts hemipteran, dipteran and/or lepidopteran insects is any of those listed above.

Suitably, the plant or part thereof which suffers from hemipteran insect infestations, or which attracts hemipteran insects, is any of those listed above.

Suitably, the plant or part thereof which suffers from blattodea insect infestations, or which attracts blattodea insects, is any of those listed above.

Suitably, the plant or part thereof which suffers from dipteran insect infestations, or which attracts dipteran insects, is any of those listed above.

Suitably, the plant or part thereof which suffers from lepidopteran insect infestations, or which attracts lepidopteran insects, is any of those listed above.

In one embodiment, the plant or part thereof is selected from a plant which suffers from or attracts hemipteran insect infestations, for example: cereal crops such as wheat (Triticum spp.), oats (Avena spp), rye (Secale spp.), barley (Hordeum spp.), rice (Oryza spp.) and corn (Zea spp.); fruit and vegetable crops including apples (Malus spp); pears (Pyrus spp); strawberry (Fragaria spp.), blueberry (Vaccinum spp.), blackberry (Rubus spp.), raspberry (Rubus spp.), citrus (Citrus spp.), olive (Olea spp.), durian (Durio spp.), longan (Dimocarpus spp.), litchi (L. chinensis), persimmon (Diospyros spp.); beans and peas (including but not limited to Phaseolus, Vigna, Pisum, Lens, Glycine, Cicer, Cajanus, Arachis spp), sugar beet (Beta vulgaris), sugar cane (Saccharum spp.), lettuce (Lactuca spp.), brassicas (Brassica spp.) including oil seed rape, alliums (Allium spp.), tomato (Solanum spp.), pepper (Capsicum spp.), asparagus (A. officinalis), melon, squash, pumpkins (Cucumis spp.), and tubers (potato) (Solanum spp.), or a part thereof.

In one embodiment, the plant or part thereof is selected from a plant which suffers from or attracts aphid insect infestations, suitably M. persicae insect infestations, including Solanaceae, Cruciferae, and Leguminosae for example: cereal crops such as wheat (Triticum spp including winter wheat Triticum aestivum L); fruit and vegetable crops including peach (Prunus spp.), strawberry (Fragaria spp.), blueberry (Vaccinum spp.), blackberry (Rubus spp.), raspberry (Rubus spp.), brassicas (Brassica spp.) such as oil seed rape, lettuce (Lactuca spp.), tomato (Solanum spp.), pepper (Capsicum spp.), beans and peas (including but not limited to Vigna, Pisum spp), melon, squash, pumpkins (Cucumis spp.), citrus (Citrus spp.), and tubers (potato) (Solanum spp.), or a part thereof. In one embodiment, the plant is a vegetable crop, suitably a Brassica spp.

In one embodiment, the plant or part thereof is selected from a plant which suffers from or attracts dipteran insect infestations, for example: cereals (Triticum spp.); oats (Avena spp); rye (Secale spp.); barley (Hordeum spp,) rice (Oryza spp.) and corn (Zea spp.); beans and peas (including but not limited to Phaseolus, Vigna, Pisum, Lens, Glycine, Cicer, Cajanus, Arachis spp); fruit crops including apples (Malus spp), pears (Pyrus spp), strawberry (Fragaria spp.), blueberry (Vaccinum spp.), blackberry (Rubus spp.), raspberry (Rubus spp.), cherry, plum, apricot, peach, nectarine (Prunus spp.), blackcurrant, redcurrant, whitecurrant, gooseberry (Ribes spp.), kiwi fruit (Actinidia spp), papaya (Carica spp.), avocado (Persea spp.), mango (Mangifera indica L), longan (Dimocarpus spp.), litchi (L. chinensis), grapes (Vitis spp.), fig (Ficus spp.), passionfruit (Passiflora spp.), Asian pears (Pyrus spp), citrus (Citrus spp.), and olive (Olea spp.); vegetable crops including alliums (Allium spp.), aubergine, tomato (Solanum spp.) and peppers (Capsicum spp.), lettuce (Lactuca spp.), brassicas (Brassica spp.) and courgette, melon, squash, pumpkins (Cucumis spp.); Apiaceae root crops including carrot (Daucus spp.), parsnip (Pastinaca spp.), or a part thereof.

In one embodiment, the plant or part thereof is selected from a plant which suffers from or attracts lepidoptera insect infestations, for example: cereal crops such as wheat (Triticum spp.), oats (Avena spp), rye (Secale spp.), barley (Hordeum spp.), rice (Oryza spp.) and corn (Zea spp.); fruit and vegetable crops including apples (Malus spp); pears (Pyrus spp); tree nuts (including for example almonds (P. amygdalus), pistachio (Pistacia vera), walnuts (Juglandaceae), hazlenuts (Corylus)); avocado, including Persea Americana (Lauraceaea), blueberry (Vaccinum spp.), citrus (Citrus spp.), olive (Olea spp.), durian (Durio spp.), longan (Dimocarpus spp.), litchi (L. chinensis), persimmon (Diospyros spp.); beans and peas (including but not limited to Phaseolus, Vigna, Pisum, Lens, Glycine, Cicer, Cajanus, Arachis spp), sugar beet (Beta vulgaris), sugar cane (Saccharum spp.), lettuce (Lactuca spp.), brassicas (Brassica spp.) including oil seed rape, alliums (Allium spp.), tomato (Solanum spp.), pepper (Capsicum spp.), asparagus (A. officinalis), melon, squash, pumpkins (Cucumis spp.), and tubers (potato) (Solanum spp.), or a part thereof.

### Insects

The compounds, compositions, combinations and agents of the invention suitably have activity against insects as described above. Preferably, the compounds, compositions, combinations and agents of the invention suitably have activity against hemipteran, dipteran, blattodea or lepidopteran insects, more preferably hemipteran, dipteran, or lepidopteran insects. However, the compounds may also have activity against other insects described herein.

The compounds, compositions, and agents of the invention may be effective against any insects. Such as Arthropoda, in particular from the class of the arachnids, for example Acarus spp., Aceria sheldoni, Aculops spp., Aculus spp., Amblyomma spp., Amphitetranychus viennensis, Argas spp., Boophilus spp., Brevipalpus spp., Bryobia praetiosa, Centruroides spp., Chorioptes spp., Dermanyssus gallinae, Dermatophagoides pteronyssius, Dermatophagoides farinae, Dermacentor spp., Eotetranychus spp., Epitrimerus pyri, Eutetranychus spp., Eriophyes spp., Halotydeus destructor, Hemitarsonemus spp., Hyalomma spp., Ixodes spp., Latrodectus spp., Loxosceles spp., Metatetranychus spp., Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vaejovis spp., Vasates lycopersici.

Still other examples are from the order of the Anoplura (Phthiraptera), for example, Damalinia spp., Haematopinus spp., Linognathus spp., Pediculus spp., Ptirus pubis, Trichodectes spp.

Still other examples are from the order of the Chilopoda, for example, Geophilus spp., Scutigera spp.

Still other examples are from the order of the Collembola, for example, Onychiurus armatus.

Still other examples are from the order of the Diplopoda, for example, Blaniulus guttulatus.

Still other examples are from the order of the Diptera, for example, Aedes spp., Agromyza spp., Anastrepha spp., Anopheles spp., Asphondylia spp., Bactrocera spp., Bibio hortulanus, Calliphora erythrocephala, Ceratitis capitata, Chironomus spp., Chrysomyia spp., Chrysops spp., Cochliomyia spp., Contarinia spp., Cordylobia anthropophaga, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopota spp., Hydrellia spp., Hylemyia spp., Hyppobosca spp., Hypoderma spp., Liriomyza spp., Lucilia spp., Lutzomia spp., Mansonia spp., Musca spp., Nezara spp., Oestrus spp., Oscinella frit, Pegomyia spp., Phlebotomus spp., Phorbia spp., Phormia spp., Prodiplosis spp., Psila rosae, Rhagoletis spp., Sarcophaga spp., Simulium spp., Stomoxys spp., Tabanus spp., Tannia spp., Tetanops spp., Tipula spp. Still other examples are from the order of the Heteroptera, for example, Anasa tristis, Antestiopsis spp., Boisea spp., Blissus spp., Calocoris spp., Campylomma livida, Cavelerius spp., Cimex spp., Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp. Still other examples are from the order of the Homoptera, for example, Acyrthosipon spp., Acrogonia spp., Aeneolamia spp., Agonoscena spp., Aleurodes spp., Aleurolobus barodensis, Aleurothrixus spp., Amrasca spp., Anuraphis cardui, Aonidiella spp., Aphanostigma pin, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Drosicha spp., Dysaphis spp., Dysmicoccus spp., Empoasca spp., Eriosoma spp., Erythroneura spp., Euscelis bilobatus, Ferrisia spp., Geococcus coffeae, Hieroglyphus spp., Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes spp., Trioza spp., Typhlocyba spp., Unaspis spp., Viteus vitifolii, Zygina spp.

Still other examples are from the order of the Hymenoptera, for example, Acromyrmex spp., Athalia spp., Atta spp., Diprion spp., Hoplocampa spp., Lasius spp., Monomorium pharaonis, Solenopsis invicta, Tapinoma spp., Vespa spp.

Still other examples are from the order of the Isopoda, for example, Armadillidium vulgare, Oniscus asellus, Porcellio scaber.

Still other examples are from the order Blattodea, for example B. germanica, Blatta orientalis, Periplaneta America, Periplaneta spp., Supella longipalpa and Supella longipalpa; also those of the family termitidae.

Still other examples are from the order of the Isoptera, for example, Coptotermes spp., Cornitermes cumulans, Cryptotermes spp., Incisitermes spp., Microtermes obesi, Odontotermes spp., Reticulitermes spp.

Still other examples are from the order of the Lepidoptera, for example, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Chematobia brumata, Chilo spp., Choristoneura spp., Clysia ambiguella, Cnaphalocerus spp., Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharina, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamestra brassicae, Mods spp., Mythimna separata, Nymphula spp., Oiketicus spp., Oria spp., Orthaga spp., Ostrinia spp., Oulema oryzae, Panolis flammea, Parnara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudoplusia includens, Pyrausta nubilalis, Rachiplusia nu, Schoenobius spp., Scirpophaga spp., Scotia segetum, Sesamia spp., Sparganothis spp., Spodoptera frugiperda, Spodoptera spp., Stathmopoda spp., Stomopteryx subsecivella, Synanthedon spp., Tecia solanivora, Thermesia gemmatalis, Tinea pellionella, Tineola bisselliella, Tortrix spp., Trichophaga tapetzella, Trichoplusia spp., Tuta absoluta, Virachola spp.

Still other examples are from the order of the Orthoptera, for example, Acheta domesticus, Dichroplus spp., Gryllotalpa spp., Leucophaea maderae, Locusta spp., Melanoplus spp., Pulex irritans, Schistocerca gregaria.

Still other examples are from the order of the Siphonaptera, for example, Ceratophyllus spp., Ctenocephalides spp., Tunga penetrans, Xenopsylla cheopis.

Still other examples are from the order of the Symphyla, for example, Scutigerella spp.

Still other examples are from the order of the Thysanoptera, for example, Anaphothrips obscurus, Baliothrips biformis, Drepanothris reuteri, Enneothrips flavens, Frankliniella spp., Heliothrips spp., Hercinothrips femoralis, Rhipiphorothrips cruentatus, Scirtothrips spp., Taeniothrips cardamoni, Thrips spp. Still other examples are from the order of the Zygentoma (=Thysanura), for example, Lepisma saccharina, Thermobia domestica. for example Lepisma saccharina, Thermobia domestica.

### Hemipteran Insects

The compounds and compositions of the invention suitably have activity against insects of the Order Hemiptera, which comprises groups including aphids, planthoppers, leafhoppers, stink bugs, shield bugs and cicadas. Suitably, the compounds, compositions and agents of the invention suitably have activity against aphids.

Hemipterans are defined by distinctive mouthparts in the form of a “beak”, comprising modified mandibles and maxillae which form a “stylet”, sheathed within a modified labium.

Many insects within these groups have endogenous neuropeptides with sequence homology to the peptides described herein, suggesting that these analogues may have activity against those insects.

The insects may belong to the sub-order Sternorrhyncha, e.g. to the superfamily of Aphidoidea (aphid superfamily), Aleyrodoidea (whiteflies), Coccoidea (scale insects), Phylloxeroidea (including Phylloxeridae or “phylloxerans”, and Adelgidae or woolly conifer aphids) or Psylloidea (jumping plant lice etc.).

Thus, the insects may be aphids, i.e. members of the aphid superfamily (Aphidoidea). Aphids (Hemiptera: Aphididae) are one of the most significant groups of agricultural pests³⁸ and are vectors in the transmission of approximately 50% of all insect transmitted plant viruses.³⁹ Within that superfamily, the aphids may be part of the family Aphididae, which contains sub-families Aiceoninae, Anoeciinae, Aphidinae, Baltichaitophorinae, Calaphidinae, Chaitophorinae, Drepanosiphinae, Eriosomatinae, Greenideinae, Hormaphidinae, Israelaphidinae, Lachninae, Lizeriinae, Macropodaphidinae, Mindarinae, Neophyllaphidinae, Phloeomyzinae, Phyllaphidinae, Pterastheniinae, Saltusaphidinae, Spicaphidinae, Taiwanaphidinae, Tamaliinae and Thelaxinae.

The aphids may, for example, be of the genus Acyrthosiphon (e.g. Acyrthosiphon pisum), Aphis (e.g. Aphis gossypii, Aphis glycines), Diuraphis (e.g. Diuraphis noxia) Macrosiphum (e.g. Macrosiphum rosae, Macrosiphum euphorbiae), Myzus (e.g. Myzus persicae), Rhopalosiphum (e.g. Rhopalosiphum padi), or Sitobion (e.g. Sitobion avenae).

Myzus persicae (peach potato aphid) is the most economically important aphid crop pest worldwide, with a global distribution and host range encompassing more than 400 species in 40 different plant families. For example, it is a major pest of agricultural crops including fruit and potatoes, and act as a vector for viruses.

Macrosiphum rosae, (rose aphid) is an important horticultural pest, especially of cultivated species of Rosa, and is a vector in the transmission of 12 plant viruses including the strawberry mild yellow edge virus.

Aphis gossypii (cotton or melon aphid) is a pest of Curcibitae and cotton.

Other than aphids, the insects may, for example, be of the Adelgidae family, e.g. of the genus Adelges (e.g. Adelges tsugae).

The insects may be of the Aleyrodidae family, e.g. of the genus Bemisia (e.g. Bemisia tabaci) or Trialeurodes (e.g. Trialeurodes vaporariorum).

The insects may be of the Psylloidea family, e.g. of the genus Pachypsylla (e.g. Pachypsylla venusta).

As examples of hemipteran insects outside the sub-order Sternorryncha, the insects may be of the Cimicidae family, e.g. of the genus Cimex (bed bugs), e.g. Cimex lectularius.

The insects may be of the Cicadellidae family, e.g. of the genus Cuerna (e.g. Cuerna arida), Graminella (e.g. Graminella nigrifrons) or Homalodisca (e.g. Homalodisca vitripennis). Also included in the Cicadellidae family are Amrasca biguttula.

The insects may be part of the Delphacidae family, e.g. of the genus Nilaparvata (e.g. Nilaparvata lugens) or Sogatella (e.g. Sogatella furcifera). For example, Nilaparvata lugens (brown planthopper) is a pest of rice crops, especially in Asia.

The insects may be of the Liviidae family, e.g. of the genus Diaphorina (e.g. Diaphorina citri).

The insects may be part of the Miridae family, e.g. of the genus Pseudatomoscelis (e.g. Pseudatomoscelis seriatus), Lygus (e.g. Lygus hesperus) or Tupiocoris (e.g. Tupiocoris notatus). For example, Pseudatomoscelis seriatus (cotton fleahopper) is a pest of cotton.

The insects may be of the Pentatomidae family, e.g. of the genus Acrosternum (e.g. Acrosternum hilare), Banasa (e.g. Banasa dimiata), Euschistus (e.g. Euschistus servus, Euschistus heroes), Halyomorpha(e.g. Halyomorpha halys), Murgantia (e.g. Murgantia histrionica), Nezara (e.g. Nezara viridula), Plautia (e.g. Plautia stali), or Podisus (e.g. Podisus maculiventris). For example, Acrosternum hilare (green stink bug) is a significant pest of cotton. Euschistus servus (brown stink bug) is a pest of many agricultural crops including seeds, grains, nuts and fruits, especially in the southern USA. Nezara viridula is a pest of grain and soybean crops, especially in Brazil.

The insects may be of the Pyrrhocoridae family, e.g. of the genus Pyrrhocoris (e.g. Pyrrhocoris apterus).

The insects may be of the Reduviidae family, e.g. of the genus Rhodnius (e.g. Rhodnius prolixus), or Triatoma (e.g. Triatoma infestans). Rhodnius prolixus is a vector of human disease (Chagas disease).

The insects may be of the Triozidae family, e.g. of the genus Acanthocasuarina (e.g. Acanthocasuarina muellerianae).

In one embodiment, the insect may be selected from the following species: H. halys, E. heroes, A. hilare, A. gossypii, E. servus, M. persicae, N. viridula, N. lugens, P. seriatus, and R. prolixus.

In one embodiment, the insect is of the species Nilaparvata lugens. In one embodiment, the compounds, compositions and agents of the invention may have activity against Nilaparvata lugens.

In one embodiment, the insect is of the species M. persicae. In one embodiment, the compounds, compositions and agents of the invention may have activity against M. persicae.

### Dipteran Insects

The compounds and compositions of the invention may have activity against insects of the Order Diptera,

In particular, they may have activity against insects of the family Drosophilidae, such as fruit flies, including those of genus Drosophila, such as Drosophila suzukii. They may also have activity against insects of the family Tephritidae, including those of the genera Anastrepha (Anastrepha spp.); Bactrocera (Bactrocera spp.); Ceratitis (Ceratitis spp.); Dacus (Dacus spp.); Rhagoletis (Rhagoletis spp.); Tephritis (Tephritis spp.).

The families Drosophilidae and Tephritidae together are commonly referred to as fruit flies.

The compounds may also have activity against other important dipteran pests, such as flies of the family Chloropidae (chloropid flies) and those of the genera:
Phytomyza (e.g. Phytomyza angelicastri);
Melani (e.g. Melani agromyza);
Antherigona (e.g. Antherigona spp);
Delia (e.g. Delia radicum);
Contarinia (e.g. Contarinia sorghicola);

For more detail on these, and other examples, see Developing the Arsenal Against Pest and Vector Dipterans: Inputs of Transgenic and Paratransgenic Biotechnologies, Ogaugwu and Durvasula, IntechOpen, 2017: DOI:10.5772/66440

### Blattodea Insects

The compounds, compositions and agents of the invention may have activity against insects of the order Blattodea, for example B. germanica, Blatta orientalis, Periplaneta America and Supella longipalpa.

In particular, they may have activity against insects of the genera Blattella.

Suitably, the compounds, compositions and agents of the invention may have activity against the species Blattella germanica.

In one embodiment, the compounds, compositions and agents of the invention may have activity against Blattella germanica.

### Lepidoptera Insects

The compounds, compositions and agents of the invention may have activity against insects of the order Lepidoptera.

In particular, they may have activity against insects of the genera: Heliothis, Plutella, Spodoptera, and Cydia. Suitably the compounds, compositions and agents of the invention may have activity against the species: Heliothis peltigera, H. virescens, Spodoptera spp., Spodoptera frugiperda, and Cydia pomonella (Codling Moth), Larvae of Heliothis spp., including peltigera and virescens Spodoptera littoralis (which represent a large variety of Heliothinae and Spodoptera moth species and are world-wide agricultural pests), and Plutella xylostella (diamondback moth, most important world-wide pest of Brassicas).

In one embodiment, the compounds, compositions and agents of the invention may have activity against Plutella xylostella.

In one embodiment, the compounds, compositions and agents of the invention may have activity against Spodoptera frugiperda.

### Household Pests

The compounds, compositions, and agents of the invention may also have activity against domestic pests, such as cockroaches and termites (such as those of the family termitidae). Of more than 3000 species, these may include the German cockroach (Blattella germanica), the Oriental cockroach (Blatta orientalis), the American cockroach (Periplaneta americana) and the brown banded cockroach (Supella longipalpa). Cockroaches are common domestic pests worldwide, and may carry various diseases. Control of these and other domestic pests (e.g. ants) is envisaged by treating surfaces where the insects run, but particularly with food baits containing compounds, compositions and agents of the invention, and with direct spray application of compounds, compositions and agents of the invention.

Therefore further aspects of the invention may include a bait comprising a compound or composition of the invention, suitably which may be a bait for domestic pests. Further aspects of the invention may include a method of controlling domestic pests, reducing domestic pest populations, inhibiting domestic pest populations, increasing domestic pest mortality, the method comprising treating a surface (e.g. wood) which is contacted by the domestic pest with a compound or composition of the invention, or contacting the domestic pest with a compound or composition of the invention. Further details of such methods are described hereinbelow. Optionally treating or contacting may comprise a suitable means of application as described elsewhere herein, such as by spraying. Suitably therefore a sprayable formulation comprising a compound or composition of the invention is also envisaged. Suitable formulations are described elsewhere herein.

### Methods and Uses of the Invention

### Methods of Increasing Insect Mortality

The invention provides a method of increasing insect mortality, comprising contacting an insect or insect population with a compound, composition or combinations as described herein. The insect or insect population may be hemipteran, dipteran and/or lepidopteran insects. The insect or insect population may also be blattodea insects.

In one embodiment, there is provided a method of increasing dipteran insect mortality, comprising contacting a dipteran insect or dipteran insect population with a compound or composition or combinations of the invention.

In one particular embodiment, there is provided a method of increasing Drosophila suzukii mortality, comprising contacting a Drosophila suzukii insect or insect population with a compound or composition or combinations of the invention.

In one embodiment, there is provided a method of increasing hemipteran insect mortality, comprising contacting a hemipteran insect or hemipteran insect population with a compound or composition as defined herein.

In one particular embodiment, there is provided a method of increasing aphid mortality, comprising contacting an aphid insect or insect population with a compound or composition or combinations as defined herein.

In one particular embodiment, there is provided a method of increasing Aphis fabae, Acyrthosiphon pisum, Myzus persicae, Amrasca biguttula or Rhopalosiphum padi mortality, comprising contacting an Aphis fabae, Acyrthosiphon pisum, Myzus persicae, Amrasca biguttula or Rhopalosiphum padi insect or insect population with a compound or composition or combinations as defined herein.

In one particular embodiment, there is provided a method of increasing Myzus persicae mortality, comprising contacting a Myzus persicae insect or insect population with compound or composition as defined herein.

In one particular embodiment, there is provided a method of increasing Rhopalosiphum padi mortality, comprising contacting a Rhopalosiphum padi insect or insect population with a compound or composition or combinations as defined herein.

In one embodiment, there is provided a method of inhibiting infestation of a plant by Nilaparvata lugens comprising contacting the plant with a compound or composition as defined herein.

In one particular embodiment, there is provided a method of increasing Nilaparvata lugens mortality, comprising contacting a Nilaparvata lugens insect or insect population with a compound or composition or combinations as defined herein.

In one particular embodiment, there is provided a method of increasing Amrasca biguttula mortality, comprising contacting a Amrasca biguttula insect or insect population with a compound or composition or combinations as defined herein.

In one embodiment, there is provided a method of increasing lepidopteran insect mortality, comprising contacting a lepidopteran insect or lepidopteran insect population with a compound or composition of the invention.

In one particular embodiment, there is provided a method of increasing Plutella xylostella mortality, comprising contacting a Plutella xylostella insect or insect population with a compound or composition as defined herein.

In one particular embodiment, there is provided a method of increasing Spodoptera frugiperda mortality, comprising contacting a Spodoptera frugiperda insect or insect population with a compound or composition as defined herein.

In one embodiment, there is provided a method of increasing Blattodea insect mortality, comprising contacting a Blattodea insect or lepidopteran insect population with a compound or composition or combinations as defined herein.

In one particular embodiment, there is provided a method of increasing Blattella germanica mortality, comprising contacting a Blattella germanica insect or insect population with a compound or composition or combinations as defined herein.

### Methods of Inhibiting or Reducing Infestation of a Plant or Site

In one embodiment, there is provided a method of inhibiting infestation of a plant by dipteran insects comprising contacting the plant with a compound or composition of the invention.

In one embodiment, there is provided a method of inhibiting infestation of a plant by hemipteran insects comprising contacting the plant with a compound or composition as defined herein.

In one particular embodiment, there is provided a method of increasing Myzus persicae mortality, comprising contacting a Myzus persicae insect or insect population with a compound or composition as defined herein.

In one embodiment, there is provided a method of inhibiting infestation of a plant by Myzus persicae comprising contacting the plant with a compound or composition as defined herein.

In one particular embodiment, there is provided a method of inhibiting infestation of a plant by Myzus persicae comprising contacting the plant with compound or composition as defined herein.

In one particular embodiment, there is provided a method of increasing Amrasca biguttula mortality, comprising contacting a Amrasca biguttula insect or insect population with a compound or composition as defined herein.

In one embodiment, there is provided a method of inhibiting infestation of a plant by Amrasca biguttula comprising contacting the plant with a compound or composition as defined herein.

In one particular embodiment, there is provided a method of increasing Halyomorph halys mortality, comprising contacting a Halyomorpha halys insect or insect population with a compound or composition as defined herein.

In one embodiment, there is provided a method of inhibiting infestation of a plant by Halyomorpha halys comprising contacting the plant with a compound or composition as defined herein.

In one particular embodiment, there is provided a method of increasing Halyomorpha halys mortality, comprising contacting a Halyomorpha halys insect or insect population with compound or composition as defined herein.

In one particular embodiment, there is provided a method of inhibiting infestation of a plant by Halyomorpha halys comprising contacting the plant with compound or composition as defined herein.

In one embodiment, there is provided a method of inhibiting infestation of a plant by lepidopteran insects comprising contacting the plant with a compound or composition of the invention.

In one embodiment, there is provided a method of inhibiting infestation of a plant by Plutella xylostella comprising contacting the plant with a compound or composition as defined herein.

In one particular embodiment, there is provided a method of increasing Plutella xylostella mortality, comprising contacting a Plutella xylostella insect or insect population with compound or composition as defined herein.

In one particular embodiment, there is provided a method of inhibiting infestation of a plant by Plutella xylostella comprising contacting the plant with compound or composition as defined herein.

In one embodiment, there is provided a method of inhibiting infestation of a plant by Spodeptera frugiperda comprising contacting the plant with a compound or composition as defined herein.

In one particular embodiment, there is provided a method of increasing Plutella xylostella mortality, comprising contacting a Spodeptera frugiperda insect or insect population with compound or composition as defined herein.

In one particular embodiment, there is provided a method of inhibiting infestation of a plant by Spodeptera frugiperda comprising contacting the plant with compound or composition as defined herein.

Suitably, contacting may comprise feeding or spraying, for example. Suitably feeding may be encouraged via bait attractants, which may be comprised in a composition of the invention, as explained below.

The methods of the invention may further comprise contacting the insect population or plant with a further insecticide agent, such as those described herein.

Suitably, the insecticide is a further kinin analogue.

### Applications of the Compounds of the Present Invention

The invention provides a method of increasing insect mortality, comprising contacting an insect or insect population with a compound as described herein. The insect or insect population may be insects of the order diptera, hemiptera or lepidoptera. The insect or insect population may be insects of the order blattodea.

The invention further provides a method of decreasing insect feeding, comprising contacting an insect or insect population with a compound as described herein. Suitably decreasing insect feeding on a plant or plant part. The insect or insect population may be Drosophila insects.

The compound may be applied directly to an insect or insect population. For example, it may be applied topically. Alternatively, the compound may be applied indirectly. For example, it may be applied to a substrate likely to come into contact with an insect or insect population. The substrate may be a plant or plant part, especially for Hemiptera or Diptera which represent pests of plants (whether crops or horticultural plants).

However, for insects which represent pests to humans, such as the Cimicidae family (e.g. bedbugs of the genus Cimex, such as Cimex lectularius) or the Reduviidae family (e.g. of the genus Rhodnius such as Rhodnius prolixus, or Triatoma such as Triatoma infestans) which can be vectors of human disease, the substrate may be a domestic surface or article, such as bedding, a mattress, or any other suitable domestic surface (e.g. wood). The compound may be applied to the substrate in a form suitable for ingestion by an insect.

The invention further provides the use of a compound as described as a plant protection agent, and specifically for protecting a plant or plant part against hemipteran, dipteran, blattodea and/or lepidopteran insects, preferably hemipteran, dipteran and/or lepidopteran insects.

The invention further provides a method of inhibiting infestation of a plant or plant part by hemipteran, dipteran, blattodea and/or lepidopteran insects, preferably hemipteran, dipteran and/or lepidopteran insects, comprising contacting the plant or plant part with a compound as described.

The method may be prophylactic. Thus, for example, the compound may be applied to the plant or plant part while the plant or part is free or substantially free of hemipteran, dipteran, blattodea and/or lepidopteran insects, preferably hemipteran, dipteran and/or lepidopteran insects.

Alternatively, the plant or plant part may already be colonised or infested by insects, such as hemipteran, dipteran, blattodea and/or lepidopteran insects, preferably hemipteran, dipteran and/or lepidopteran insects. Thus, the invention further provides a method of reducing infestation of a plant or plant part, or of reducing insect, e.g. hemipteran, dipteran and/or lepidopteran insect load on a plant or plant part, the method comprising contacting the plant or plant part with a compound as described.

In any of these embodiments, the compound may be provided as part of a composition, such as an insect control composition (e.g. insecticide composition) or a plant protection composition. Reference to application or use of a compound should therefore be construed as encompassing application or use of a suitable composition, unless the context demands otherwise.

The composition typically comprises a compound as described in combination with one or more ancillary component such as solvents, carriers, diluents, adjuvants, preservatives, dispersants, emulsifying agents, or synergists.

The composition may further comprise one or more additional active insecticides described herein.

The invention further provides a composition, e.g. an insect control composition or plant protection composition, comprising a compound of the invention in admixture with one or more solvents, carriers, diluents, adjuvants, preservatives, dispersants, emulsifying agents, or synergists. The composition may be an aqueous composition.

The method may be prophylactic. Thus, for example, the compound may be applied to the plant or plant part, or site, while the plant or part or field is free or substantially free of insects.

Suitably the site may be any agricultural site suitable for growing plants. Suitably the site may be any area or locale which may be suitable for growing plants, or in which plants are grown. Suitable sites may include farmland, brownfield sites, fields, greenhouses, conservatories, containers, aquaponics and hydroponics systems etc. In one embodiment, the site is a field.

Alternatively, the plant or plant part or site may already be colonised or infested by insects, suitably by hemipteran, dipteran, blattodea and/or lepidopteran insects.

Thus, the invention further provides a method of reducing insect infestation of a plant or plant part, or of reducing insect load on a plant or plant part, the method comprising contacting the plant or plant part with a compound, composition, or combinations as described herein. The invention also provides a method of reducing insect infestation of a field, or of reducing an insect load in a field, the method comprising contacting the field with a compound, composition, or combinations as described herein. Suitably of hemipteran, dipteran, blattodea and/or lepidopteran insects.

### Methods of Decreasing Insect Feeding

The invention further provides a method of decreasing insect feeding, comprising contacting an insect or insect population with a compound, composition or combinations as described herein. Suitably decreasing insect feeding on a plant or plant part. The insect or insect population may be hemipteran, dipteran, blattodea and/or lepidopteran insects.

Methods of Protecting a Plant from Insects

The invention further provides a method of protecting a plant or part thereof from insects, or from insect infestation, and specifically a method of protecting a plant or plant part against hemipteran, dipteran, blattodea and/or lepidopteran insects or infestation thereof, wherein the method comprises the step of applying directly or indirectly to the plant or to a part of the plant, an insecticidal compound or composition, or combinations of the invention. Suitably applying indirectly may comprise applying the compound or composition or combinations of the invention to the field or locale in which the plant is growing, or in which it is intended to be grown.

The present invention also provides post-harvest treatment methods for protecting or treating a harvested plant or a harvested part of the plant against insects, or insect infestation, specifically against hemipteran, dipteran, blattodea and/or lepidopteran insects or infestation thereof, the method comprising the step of applying directly or indirectly to the harvested plant or to a harvested part of the plant, an insecticidal compound or composition or combinations of the invention, under conditions effective to protect or treat the harvested plant or a harvested part of the plant against the insects. Suitably applying indirectly may comprise applying the compound or composition or combinations of the invention to the location in which the plant or a harvested part of the plant is stored, or in which it is intended to be stored.

Suitably the insect may be of the order hemipteran, dipteran, blattodea and/or lepidopteran.

The present invention provides the use of a compound as described herein as an insect control agent, specifically in methods of increasing mortality in insects that encode the kinin peptide, or a method of inhibiting infestation of a plant by insects that encode the kinin peptide.

### Methods of Contacting an Insect

In one embodiment, there is provided a method of contacting an insect with a compound, preferably an insect control agent, even more preferably an insecticide, said method comprising applying to or on sites frequented by the insect a compound or composition or combinations according to the invention.

The site, frequented by insects may be a natural habitat for insects, or a place regularly visited by insects. This site can be treated then with the compound or composition as described above: as a non-limiting example mosquito nets, impregnated with encapsulated insecticide, can be used as application method. Alternatively, said site is created by application of a visual lure or of an attractant for the insects. Visual lures are known to the person skilled in the art, and include but are not limited to light sources, coloured object and shapes or silhouettes that stand out of a contrasting background. As mentioned above, insect attractants include but are not limited to pheromones, kairomones and allomones. The attractant may be present in the composition or it may be applied separately from the compound or composition or combinations, to ensure that the insects are attracted to the site where the compound or composition or combinations is applied.

Suitably the insect may be of the order hemipteran, dipteran, blattodea and/or lepidopteran.

### Particular Embodiments of the Invention

In a particular embodiment, compounds of Formula (Ia) find particular use as insecticides against dipteran insects (e.g. D. suzukii). Suitably, the compound of Formula (Ia) is selected from one or more of the compounds in the table below:

| Reference | | |
|---|---|---|
| Number | Sequence | SEQ ID NO |
| SB-P-032 | Ac-KQrFH[Aib]WG-[NH₂] | SEQ ID NO: 18 |
| SB-P-033 | Ac-NSVVLGKKQrFH[Aib]WG-[NH₂] | SEQ ID NO: 19 |
| SB-P-034 | Ac-FH[Aib]WG-[NH₂] | SEQ ID NO: 20 |
| SB-P-035 | Ac-KQrFHsWG-[NH₂] | SEQ ID NO: 21 |
| SB-P-036 | Ac-FHsWG-[NH₂] | SEQ ID NO: 22 |
| SB-P-052 | Hy-NSVVLGKKQ[n-me-R]FH[n-me-S]WG-[NH₂] | SEQ ID NO: 23 |

or salts or solvates thereof.

In a particular embodiment, compounds of Formula (Ib) find particular use as insecticides against insects of the order hemiptera (for example Halyomorpha halys and Myzus persicae species). Suitably, the compound of Formula (Ib) is selected from one or more of the compounds in the table below

| Reference | | |
|---|---|---|
| number | Sequence | SEQ ID NO |
| SB-P-003 | Hy-DPKYKFSSWG-[NH₂] | SEQ ID NO: 59 |
| SB-P-004 | Hy-AKFSSWG-[NH₂] | SEQ ID NO: 60 |
| SB-P-018 | Hy-ARFSSWA-[NH₂] | SEQ ID NO: 61 |
| SB-P-019 | Hy-SKFNSWA-[NH₂] | SEQ ID NO: 62 |
| SB-P-020 | Hy-AKFSSWA-[NH₂] | SEQ ID NO: 63 |
| SB-P-021 | Hy-ARFNSWA-[NH₂] | SEQ ID NO: 64 |
| SB-P-022 | Hy-VPFNSWA-[NH₂] | SEQ ID NO: 65 |
| SB-P-023 | Hy-PIFSSWG-[NH₂] | SEQ ID NO: 66 |
| SB-P-024 | Hy-GPDFYAWG-[NH₂] | SEQ ID NO: 67 |
| SB-P-054 | Ac-SkFN-Aib-WA-[NH₂] | SEQ ID NO: 68 |
| SB-P-056 | Ac-SK-[n-me-F]-N-(α-Me)Ser-WA-[NH₂] | SEQ ID NO: 69 |
| SB-P-060 | Ac-PA-[n-me-F]-SsWG-[NH₂] | SEQ ID NO: 70 |
| SB-P-065 | Hy-RQKTVFSSWG-[NH₂] | SEQ ID NO: 71 |
| SB-P-066 | Hy-PAFSSWG-[NH₂] | SEQ ID NO: 72 |
| SB-P-067 | ASDKHGRPKQTFSSWG-[NH₂] | SEQ ID NO: 73 |
| SB-P-100 | Ac-PA-[n-me-F]-S-[βA]-WG-[NH₂] | SEQ ID NO: 74 |
| SB-P-101 | Ac-PA-[n-me-F]-S-(α-Me)S-WG-[NH₂] | SEQ ID NO: 75 |

or salts or solvates thereof.

In a particular embodiment, compounds of Formula (Ic) find particular use as insecticides against lepidoptera insects (e.g. Plutella xylostella or Spodoptera frugiperda). Suitably, the compound of Formula (Ib) is selected from one or more of the compounds in the table below

| Reference | | |
|---|---|---|
| Number | Sequence | SEQ ID NO |
| SB-P-068 | Hy-VRFSPWG-[NH₂] | SEQ ID NO: 107 |
| SB-P-069 | Hy-NFSPWG-[NH₂] | SEQ ID NO: 108 |
| SB-P-070 | Hy-KVKFSAWG-[NH₂] | SEQ ID NO: 109 |
| SB-P-071 | Hy-SFSPWG-[NH₂] | SEQ ID NO: 110 |
| SB-P-072 | Hy-FSPWG-[NH₂] | SEQ ID NO: 111 |
| SB-P-073 | Hy-YFSPWG-[NH₂] | SEQ ID NO: 112 |
| SB-P-074 | Hy-KVKFSVWG-[NH₂] | SEQ ID NO: 113 |
| SB-P-075 | Hy-KVKFSTWG-[NH₂] | SEQ ID NO: 114 |
| SB-P-076 | Hy-VRFSPWG-[NH₂] | SEQ ID NO: 115 |
| SB-P-102 | Ava-FS-BA-WG-[NH₂] | SEQ ID NO: 116 |
| SB-P-103 | Ava-FS-Thz-WG-[NH₂] | SEQ ID NO: 117 |

or salts or solvates thereof.

In a particular embodiment, the compound for use against insects of the order Lepidoptera (e.g. Spodoptera frugiperda) is selected from one of the compounds in the table below:

| | | |
|---|---|---|
| SB-P-068 | Hy-VRFSPWG-[nh2] | SEQ ID NO: 107 |
| SB-P-070 | Hy-KVKFSAWG-[nh2] | SEQ ID NO: 109 |
| SB-P-073 | Hy-YFSPWG-[NH₂] | SEQ ID NO: 112 |

or a salt or a solvate thereof.

In a particular embodiment, the compound for use against insects of the order Hemiptera (e.g. Nilaparvata lugens) is selected from one of the compounds in the table below:

| | | |
|---|---|---|
| N/A | Hy-GPAFSSWG-[NH₂] | SEQ ID NO: 92 |
| N/A | Hy-AAAFNSWG-[NH₂] | SEQ ID NO: 93 |
| N/A | Hy-NAAFSSWG-[nh2] | SEQ ID NO: 94 |
| SB-P-066 | Hy-PAFSSWG-[NH₂] | SEQ ID NO: 72 |
| N/A | Hy-NAAFNSWG-[NH₂] | SEQ ID NO: 95 |
| N/A | Hy-VPAFNSWG-[NH₂] | SEQ ID NO: 96 |
| N/A | Hy-TNTAFSSWG-[NH₂] | SEQ ID NO: 97 |
| N/A | Hy-PRFYSWG-[NH₂] | SEQ ID NO: 98 |
| N/A | Hy-GADFYAWG-[NH₂] | SEQ ID NO: 99 |

or a salt or a solvate thereof.

In a particular embodiment, the compound for use against insects of the order Blattodea (e.g. Blattella germanica) is selected from one of the compounds in the table below:

| | | |
|---|---|---|
| SB-P-143 | Hy-DPAFNSWG-[NH₂] | SEQ ID NO: 76 |
| SB-P-144 | Hy-SSAFNSWG-[NH₂] | SEQ ID NO: 77 |
| SB-P-145 | Hy-RAFTSTGSSRSPAFSSWG-[NH₂] | SEQ ID NO: 78 |
| SB-P-146 | Hy-PASFSSWG-[NH₂] | SEQ ID NO: 79 |
| SB-P-147 | Hy-DAGFSSWG-[NH₂] | SEQ ID NO: 80 |
| SB-P-148 | Hy-SSGFSSWG-[NH₂] | SEQ ID NO: 81 |
| SB-P-149 | Hy-DPVFKSWG-[NH₂] | SEQ ID NO: 82 |
| SB-P-150 | Hy-DAAFSSWG-[NH₂] | SEQ ID NO: 83 |
| SB-P-151 | Hy-GSGFSSWG-[NH₂] | SEQ ID NO: 84 |
| SB-P-154 | Hy-DDDSDVSESGSKFSSWG-[NH₂] | SEQ ID NO: 85 |
| SB-P-155 | Hy-SFSSWG-[NH₂] | SEQ ID NO: 86 |
| SB-P-156 | Hy-GFSSWG-[NH₂] | SEQ ID NO: 87 |
| SB-P-157 | Hy-LDRSEGRVSKALFSSWG-[NH₂] | SEQ ID NO: 88 |
| SB-P-158 | Hy-WGQSSVGAVFSSWG-[NH₂] | SEQ ID NO: 89 |
| SB-P-159 | Hy-GAEFYSWG-[NH₂] | SEQ ID NO: 90 |

or a salt or a solvate thereof.

### Application of the Compounds or Compositions

The compound or composition or combinations of the invention may be contacted with the insect or insect population, suitably this may comprise applying the compound or composition or combinations directly to an insect or insect population. For example, it may be applied topically. Alternatively, the compound or composition or combinations may be applied indirectly. For example, it may be applied to a substrate, site, or locale likely to come into contact with an insect or insect population. The substrate may be a plant or plant part, especially for Hemiptera or Diptera or Lepidoptera which represent pests of plants (whether crops or horticultural plants), or may be a field, locale, or area suitably in which the plants are grown. Suitably therefore the compound or composition or combinations may be applied to the plant or plant part. Suitably therefore the compound or composition or combinations may be applied to the site in which the plant is grown.

However, for insects which represent pests to humans, such as the Cimicidae family (e.g. bedbugs of the genus Cimex, such as Cimex lectularius) or the Reduviidae family (e.g. of the genus Rhodnius such as Rhodnius prolixus, or Triatoma such as Triatoma infestans) which can be vectors of human disease, the substrate may be a domestic surface or article, such as bedding, a mattress, or any other suitable domestic surface. The compound or composition may be applied to the substrate in a form suitable for ingestion by an insect.

Suitably contacting may comprise feeding or spraying, for example. Suitably feeding may be encouraged via bait attractants, which may be comprised in a composition of the invention, as explained below.

The methods may comprise contacting or applying directly or indirectly to the plant or to a part of the plant a compound or composition or combinations as disclosed herein, for example at an application rate higher than 5 g of the compound per hectare, such as but not limited to an application rate higher than 10 g of the compound per hectare, such as an application rate higher than 24 g of the compound per hectare, such as an application rate higher than 50 g of the compound per hectare, such as an application rate higher than 75 g of the compound per hectare, such as an application rate higher than 100 g of the compound per hectare, or in particular an application rate higher than 200 g of the compound per hectare. These methods may comprise applying directly or indirectly to the plant or to a part of the plant a compound, composition or combinations as disclosed herein, for example at an application rate between 5 g and 100 g of the compound composition or combinations per hectare, such as but not limited to an application rate of between 5 g and 200 g of the compound composition or combinations per hectare, in particular an application rate of between 5 g and 50 g of the compound composition or combinations per hectare, such as between 5 g and 30 g of the compound composition or combinations per hectare or between 10 g and 25 g per hectare.

The compounds or compositions or combinations as disclosed herein may be directly or indirectly contacted with/applied to the plant or to a part of the plant by spraying, atomizing, foaming, fogging, culturing in hydroculture, culturing in hydroponics, coating, submerging, and/or encrusting, optionally post-harvest. Suitably contacting may comprise feeding or spraying, for example. In some embodiments, the contacting is by feeding.

Suitably the compound may be contacted with the insect or insect population, or plant or plant part, at any suitable concentration which is effective. Suitably the compositions and combinations may comprise such an effective concentration of a compound. Suitably the concentration of the compound is between 10⁻³ to 10⁻⁹ M, suitably between 10⁻⁴ to 10⁻⁶ M, suitably between 10⁻⁴ to 10⁻⁵ M.

### Use as a Plant Protection Agent

The invention further provides the use of a compound, composition or combination as described herein as a plant protection agent, and specifically for protecting a plant or plant part against insects, suitably against hemipteran, dipteran, blattodea and/or lepidopteran insects, more suitably against hemipteran, dipteran and/or lepidopteran insects. Plant protection agents are described further hereinabove.

### Use as an Insect Control Agent

The present invention provides the use of a compound or composition or combination thereof as described herein as an insect control agent, specifically in methods of increasing mortality in insects, or a method of inhibiting infestation of a plant by insects.

The present invention also provides the use of a compound as described herein as an insect control agent, specifically in methods of increasing hemipteran, dipteran and/or lepidopteran insect mortality, or a method of inhibiting infestation of a plant by hemipteran, dipteran, blattodea and/or lepidopteran insects. Preferably, the present invention relates to the use of a compound as described herein as an insect control agent, specifically in methods of increasing hemipteran insect mortality, or a method of inhibiting infestation of a plant by hemipteran, dipteran, blattodea and/or lepidopteran insects.

The present invention also provides the use of a compound or composition or combination thereof as described herein as an insect control agent, specifically by having a biostatic effect on hemipteran, dipteran, blattodea and/or lepidopteran insects, a biocidal effect on hemipteran, dipteran, blattodea and/or lepidopteran insect, and/or a pesticidal effect hemipteran, dipteran, blattodea and/or lepidopteran insect.

“Biostatic (effect)” or “biostatic use”, as used herein, includes any effect or use of a compound or composition or combination as described herein (optionally comprised in a biostatic, biocidal, fungicidal or fungistatic composition as defined herein) for controlling, modulating or interfering with the harmful activity of a pest, such as a plant pest or a plant pathogen. Suitably the pest is of hemipteran, dipteran, blattodea and/or lepidopteran insect orders. Suitably including but not limited to inhibiting the growth or activity of the insect, altering the behaviour of the insect, and repelling or attracting the insect in plants, plant parts or in other agro-related settings, such as for example for household uses or in soil.

“Pesticidal activity” or “biocidal activity”, as used interchangeably herein, means killing the pest or severely disabling the pest. Suitably which may be the same as insecticidal activity wherein the pest is an insect. Suitably, the compound, composition or combination may be for use as an insect control agent wherein the insect encodes a kinin peptide.

Suitably, the compound may be for use as an insect control agent wherein the insect is of the order dipteran. Suitably, the compound may be for use as an insect control agent wherein the insect is of the genus Drosophila. Suitably, the compound may be for use as an insect control agent wherein the insect is Drosophila suzukii.

In some embodiments, the compound for use against insects of the order diptera is any one of the specific compounds described herein.

Suitably, the compound or composition as defined herein may be for use as an insect control agent wherein the insect is of the order hemipteran.

Suitably, the compound or composition or combination as defined herein may be for use as an insect control agent wherein the insect is an aphid.

Suitably, the compound or composition as defined herein may be for use as an insect control agent wherein the insect is of the genus Myzus. Suitably, the compound or composition as defined herein may be for use as an insect control agent wherein the insect is Myzus persicae.

Suitably, the compound or composition as defined herein may be for use as an insect control agent wherein the insect is of the family Cicadellidae. Suitably, the compound or composition as defined herein may be for use as an insect control agent wherein the insect is Amrasca biguttula.

Suitably, the compound or composition as defined herein may be for use as an insect control agent wherein the insect is of the genus Halyomorpha. Suitably, the compound or composition as defined herein may be for use as an insect control agent wherein the insect is Halyomorpha halys.

In some embodiments, the compound for use against insects of the order hemiptera is any one of the specific compounds described herein.

Suitably, the compound may be for use as an insect control agent wherein the insect is of the order lepidoptera.

Suitably, the compound or composition as defined herein may be for use as an insect control agent wherein the insect is of the genus Plutella. Suitably, the compound or composition as defined herein may be for use as an insect control agent wherein the insect is Plutella xylostella.

In some embodiments, the compound for use against insects of the order lepidoptera is any one of the specific compounds described herein.

Suitably, the compound or composition or combination as defined herein may be for use as an insect control agent wherein the insect is of the order Blattodea. Suitably, the compound or composition or combination as defined herein may be for use as an insect control agent wherein the insect is a cockroach or a termite.

### Methods of Controlling Insect Fecundity

The present invention also provides insecticidal compounds, compositions and combinations as described herein for controlling fecundity of insect species. Suitably, the insect is selected from hemipteran, dipteran or lepidopteran insects, such as those described anywhere herein. Suitably wherein in some embodiments the hemipteran insect is Myzus persicae. Suitably wherein in some embodiments the dipteran insect is D. suzukii. Suitably wherein in some embodiments the lepidopteran insect is Plutella xylostella.

There is also provided a method of controlling fecundity of an hemipteran insect, the method comprising contacting a compound of the invention, or a composition of the invention to a substrate, e.g. a plant or soil. Suitably, the hemipteran insect is any of those disclosed herein, e.g., an aphid.

There is also provided a method of controlling fecundity of a dipteran insect, the method comprising contacting a compound of the invention, or a composition of the invention to a substrate, e.g. a plant or soil. Suitably, the dipteran insect is any of those disclosed herein.

There is also provided a method of controlling fecundity of an lepidopteran insect, the method comprising contacting a compound of the invention, or a composition of the invention to a substrate, e.g. a plant or soil. Suitably, the lepidopteran insect is any of those disclosed herein.

There is also provided a method of controlling fecundity of a blattodea insect, the method comprising contacting a compound of the invention, or a composition of the invention to a substrate, e.g. a plant or soil. Suitably, the blattodea insect is any of those disclosed herein.

There is also provided a method of controlling fecundity of a hemipteran insect, the method comprising contacting a combination as described herein, to a substrate, e.g. a plant or soil. Suitably, the hemipteran insect is any of those disclosed herein.

Suitably, the combination comprises a compound of the invention, and a kinin peptide, or an analogue thereof.

There is also provided a method of controlling fecundity of a dipteran insect, the method comprising contacting a combination as described herein, to a substrate, e.g. a plant or soil. Suitably, the dipteran insect is any of those disclosed herein. Suitably, the combination comprises a compound of the invention, and a kinin peptide, or an analogue thereof.

There is also provided a method of controlling fecundity of a lepidopteran insect, the method comprising contacting a combination as described herein, or a composition of the invention to a substrate, e.g. a plant or soil. Suitably, the lepidopteran insect is any of those disclosed herein. Suitably, the combination comprises a compound of the invention, and a kinin peptide, or an analogue thereof.

There is also provided a method of controlling fecundity of a blattodea insect, the method comprising contacting a combination as described herein, or a composition of the invention to a substrate, e.g. a plant or soil. Suitably, the blattodea insect is any of those disclosed herein. Suitably, the combination comprises a compound of the invention, and a kinin peptide, or an analogue thereof.

There is also provided a method of controlling fecundity of an hemipteran insect or insect population, the method comprising contacting a compound of the invention, or a composition of the invention to an hemipteran insect or insect population. Suitably, the hemipteran insect is any of those disclosed herein.

There is also provided a method of controlling fecundity of a dipteran insect or insect population, the method comprising contacting a compound of the invention, or a composition of the invention to a dipteran insect or insect population. Suitably, the dipteran insect is any of those disclosed herein.

There is also provided a method of controlling fecundity of a lepidopteran insect or insect population, the method comprising contacting a compound of the invention, or a composition of the invention to a lepidopteran insect or insect population. Suitably, the lepidopteran insect is any of those disclosed herein.

There is also provided a method of controlling fecundity of a hemipteran insect or insect population, the method comprising contacting a combination as described herein, to a hemipteran insect or insect population. Suitably, the hemipteran insect is any of those disclosed herein. Suitably, the combination comprises a compound of the invention, and a kinin peptide, or an analogue thereof.

There is also provided a method of controlling fecundity of a dipteran insect or insect population, the method comprising contacting a combination as described herein, to a dipteran insect or insect population. Suitably, the dipteran insect is any of those disclosed herein. Suitably, the combination comprises a compound of the invention, and a kinin peptide, or an analogue thereof.

There is also provided a method of controlling fecundity of a lepidopteran insect or insect population, the method comprising contacting a combination as described herein, or a composition of the invention to a lepidopteran insect or insect population.

Suitably, the lepidopteran insect is any of those disclosed herein. Suitably, the combination comprises a compound of the invention, and a kinin peptide, or an analogue thereof.

There is also provided a method of controlling fecundity of a blattodea insect or insect population, the method comprising contacting a combination as described herein, or a composition of the invention to a blattodea insect or insect population. Suitably, the blattodea insect is any of those disclosed herein. Suitably, the combination comprises a compound of the invention, and a kinin peptide, or an analogue thereof.

In the context of the present invention, “controlling fecundity” refers to reducing, inhibiting, or eliminating the presence of an insect species during one or more of its growth stages. For example, the compositions of the present invention may be used for controlling the growth of mites at any stage such as egg, larva, nymph, and adult form.

Combinations with Further Insecticides

The compound of the invention may be used in combination with one or more further insecticides, such as those as described herein. There is also provided a combination comprising a compound of the invention, or a salt or solvate thereof, in combination with one or more additional active insecticides, or insecticidal compounds.

The composition of the invention may further comprise one or more additional active insecticides, or active insecticidal compounds.

Suitably, the insecticide may be selected from an insect neuropeptide or analogues thereof. Suitably, the insecticide is a further kinin analogue, an AKH peptide, a DH31 peptide, a DH44 peptide, a pyrokinin peptide and/or a CAPA peptide (e.g. a CAPA-1, CAPA-2 or CAPA-3 peptide or analogue).

The additional active insecticide may be selected from a CAPA peptide, an AKH peptide, a DH31 peptide and/or a pyrokinin peptide.

Suitably, the insecticide may be selected from an insect neuropeptide or analogues thereof such as a further kinin peptide, a AKH peptide, a DH31 peptide, a DH44 peptide, a pyrokinin peptide or a CAPA peptide (e.g. a CAPA-1, CAPA-2 or CAPA-3 peptide or analogue).

Additionally or alternatively, the insecticide may be selected from a chemical insecticide, such as: Pyrethroids (Permethrin, Cypermethrin, Deltamethrin); Organophosphates (Malathion, Chlorpyrifos, Diazinon); Neonicotinoids (Imidacloprid, Clothianidin, Thiamethoxam); Carbamates (Carbaryl, Methomyl, Propoxur); Botanical Insecticides (Pyrethrin (derived from chrysanthemum flowers), Rotenone (derived from certain plant roots)); Biopesticides (Bacillus thuringiensis (Bt) products, Beauveria bassiana (fungus), Metarhizium anisopliae (fungus)); Insect Growth Regulators (IGRs) (Methoprene, Pyriproxyfen); Fipronil; Spinosad; Avermectins (including abamectin and ivermectin); Chitin Synthesis Inhibitors (Diflubenzuron, Hexaflumuron); Piperonyl Butoxide; Flonicamid; Indoxacarb; Chlorantraniliprole; dichlorodiphenyltrichloroethane; and Sulfoxaflor.

In a particular embodiment, the further insecticide is a further kinin peptide.

In a particular embodiment, the further insecticide is an AKH peptide.

In a particular embodiment, the further insecticide is a DH31 peptide.

In a particular embodiment, the further insecticide is a DH44 peptide,

In a particular embodiment, the further insecticide is a pyrokinin peptide,

In a particular embodiment, the further insecticide is a CAPA peptide (e.g. a CAPA-1, CAPA-2 or CAPA-3 peptide or analogue).

Suitably said further insecticides or insecticidal compounds may be comprised in a composition of the invention.

The methods of the invention may further comprise contacting the insect population or plant or part thereof with a further insecticide agent, or insecticidal compound, such as those described herein. Suitably, the further insecticide or insecticidal compound is a kinin peptide.

Suitably any references herein to the compound of the invention or a composition thereof may equally refer to a combination of a compound of the invention with one or more insecticides or insecticidal compounds, or a composition containing such a combination.

Suitably, there is provided a combination comprising a compound of the invention, or a salt or solvate thereof, in combination with one or more of the peptides listed in the table below:

| Family | Peptide | Sequence | SEQ ID NO |
|---|---|---|---|
| Pyrokinin | SB-P-46 | [Hy]-SPPYSPPFSPRL-[NH₂] | SEQ ID NO: 123 |
| | SB-P-47 | [pyr]AIMARPQVPRL-[NH₂] | SEQ ID NO: 124 |
| | SB-P-48 | Hy-EQNVQSNGEPAYRVRTPRL-[NH₂] | SEQ ID NO: 125 |
| | SB-P-49 | [Hy]-SVPFKPRL-[NH₂] | SEQ ID NO: 126 |
| | SB-P-51 | [Hy]-LRQLQSNGEPAYRVRTPRL-[NH₂] | SEQ ID NO: 127 |
| | SB-P-80 | [Hy]-NADEDQQQSVDFTPRL-[NH₂] | SEQ ID NO: 128 |
| | SB-P-81 | [Hy]-GGSMTFSPRL-[NH₂] | SEQ ID NO: 129 |
| Kinin | SB-P-70 | [Hy]-KVKFSAWG-[NH₂] | SEQ ID NO: 109 |
| | SB-P-65 | [Hy]-RQKTVFSSWG-[NH₂] | SEQ ID NO: 71 |
| | SB-P-66 | [Hy]-PAFSSWG-[NH₂] | SEQ ID NO: 72 |
| AKH | SB-P-86 | [pyr]-LTFTSSWGG-[NH₂] | SEQ ID NO: 130 |
| | SB-P-39 | [palm]-QLTFSPDW-[NH₂] | SEQ ID NO: 131 |
| | SB-P-41 | Hy-QLTFSPDW-[NH₂] | SEQ ID NO: 132 |
| | SB-P-42 | [pyr]LTFSPDW-[NH₂] | SEQ ID NO: 133 |

In one embodiment, the compound of the invention is utilised in combination with a pyrokinin peptide, e.g.;

| Family | Peptide | Sequence | SEQ ID NO |
|---|---|---|---|
| Pyrokinin | SB-P-46 | [Hy]-SPPYSPPFSPRL-[NH₂] | SEQ ID NO: 123 |
| | SB-P-47 | [pyr]AIMARPQVPRL-[NH₂] | SEQ ID NO: 124 |
| | SB-P-48 | Hy-EQNVQSNGEPAYRVRTPRL-[NH₂] | SEQ ID NO: 125 |
| | SB-P-49 | [Hy]-SVPFKPRL-[NH₂] | SEQ ID NO: 126 |
| | SB-P-51 | [Hy]-LRQLQSNGEPAYRVRTPRL-[NH₂] | SEQ ID NO: 127 |

In one embodiment, the compound of the invention is utilised in combination with a further kinin peptide, e.g.;

| Family | Peptide | Sequence | SEQ ID NO |
|---|---|---|---|
| Kinin | SB-P-70 | [Hy]-KVKFSAWG-[NH₂] | SEQ ID NO: |
| | | | 109 |
| | SB-P-65 | [Hy]-RQKTVFSSWG-[NH₂] | SEQ ID NO: |
| | | | 71 |
| | SB-P-66 | [Hy]-PAFSSWG-[NH₂] | SEQ ID NO: |
| | | | 72 |

In one embodiment, the compound of the invention is utilised in combination with a CAPA peptide (e.g. a CAPA2 peptide).

In one embodiment, the compound of the invention is utilised in combination with an AKH peptide, e.g.;

| Family | Peptide | Sequence | SEQ ID NO |
|---|---|---|---|
| AKH | SB-P-86 | [pyr]-LTFTSSWGG- | SEQ ID NO: 130 |
| | | [NH₂] | |
| | SB-P-39 | [palm]-QLTFSPDW- | SEQ ID NO: 131 |
| | | [NH₂] | |
| | SB-P-41 | Hy-QLTFSPDW-[NH₂] | SEQ ID NO: 132 |
| | SB-P-42 | [pyr]LTFSPDW-[NH₂] | SEQ ID NO: 133 |

The choice of ancillary or additional insecticides will typically depend on the particular target species.

### Specific Combinations

The additional active insecticide may be selected from a CAPA peptide, e.g. SDSKNT[n-me-A]LWFGPRL-[NH₂](SEQ ID NO: 134) (SB-P-015), ASG[b³L]VAFPRV-[NH₂](SEQ ID NO: 135) (2315), Palm-LVAFPRV-[NH₂](SEQ ID NO: 136) (AH59) or [ahx]-LV[n-me-A]FPR[n-me-V]—[NH₂](SEQ ID NO: 137) (AH270).

Suitably, the compounds of the invention may be provided in combination with one or more additional active insecticides selected from:

(SB-P-015)
(SEQ ID NO: 134)
SDSKNT[n-me-A]LWFGPRL-[NH₂]
(2315)
(SEQ ID NO: 135)
ASG[b3L]VAFPRV-[NH₂]
(AH59)
(SEQ ID NO: 136)
Palm-LVAFPRV-[NH₂]
(AH270)
(SEQ ID NO: 137)
[ahx]-LV[n-me-A]FPR[n-me-V]-[NH₂].

In a particular embodiment, there is provided a combination comprising the peptides described in the examples and in the figures, in particular:

SB-P-3:
(SEQ ID NO: 59)
DPKYKFSSWG-[NH₂]
and
SB-P-015:
(SEQ ID NO: 134)
SDSKNT[n-me-AJLWFGPRL-[NH₂];
SB-P-3:
(SEQ ID NO: 59)
DPKYKFSSWG-[NH₂]
and
2315:
(SEQ ID NO: 135)
ASG[b3L]VAFPRV-[NH₂];
SB-P-3:
(SEQ ID NO: 59)
DPKYKFSSWG-[NH₂],
SB-P-015:
(SEQ ID NO: 134)
SDSKNT[n-me-AJLWFGPRL-[NH₂]
and
2315:
(SEQ ID NO: 135)
ASG[b3L]VAFPRV-[NH₂];
SB-P-3:
(SEQ ID NO: 59)
DPKYKFSSWG-[NH₂]
and
AH59:
(SEQ ID NO: 136)
Palm-LVAFPRV-[NH₂]
SB-P-3:
(SEQ ID NO: 59)
DPKYKFSSWG-[NH₂],
SB-P-015:
(SEQ ID NO: 134)
SDSKNT[n-me-AJLWFGPRL-[NH₂]
and
AH59:
(SEQ ID NO: 136)
Palm-LVAFPRV-[NH₂];
or
SB-P-3:
(SEQ ID NO: 59)
DPKYKFSSWG-[NH₂]
and
AH270:
(SEQ ID NO: 137)
[ahx]-LV[n-me-A]FPR[n-me-V]-[NH₂];

or salts or solvates thereof.

### Beneficial Insect Species (Such as Pollinator Species)

The compounds, compositions and combinations of the invention may be substantially non-toxic to beneficial insect species, including species which prey on pests and pollinator species. Important pollinator species, such as insects of the superfamily Apoidea, including bees, such as the Apidae, e.g. those of the genus Bombus, such as Bombus terrestris. Important predatory species include Coccinellidae (lady bugs) such as Adalia bipunctata.

By substantially non-toxic, it is meant that the compounds, compositions and combinations of the invention do not cause death of the beneficial insect species (e.g. pollinator species), suitably that they do not cause premature death of the beneficial insect species (e.g. pollinator species). It is also meant that the compounds, compositions and combinations of the invention do not cause any detrimental side effects to the beneficial insect species (e.g. pollinator species), for example they do not have a negative effect on feeding behaviour, or ability to move.

### Compositions

The present inventors have provided compositions comprising at least one insecticidal compound or combination thereof of the invention, which can specifically bind to an insect. Importantly, through this interaction with a specific molecular structure of the insect, such as a receptor, suitably a neurological receptor, the compositions disclosed herein are capable of inhibiting, preventing or reducing one or more biological activities of the insect, such that the growth or fecundity of the insect is inhibited, prevented or reduced.

In certain embodiments, the compositions as disclosed herein are capable of killing insects through the specific interaction of at least one insecticidal compound, which can specifically bind to an insect receptor, and which is comprised in the compositions.

Compositions of the invention, or for use in accordance with the invention, typically comprise a compound as described in combination with one or more ancillary component such as solvents, carriers, diluents, adjuvants, preservatives, dispersants, emulsifying agents, or synergists.

Suitably the composition may be an agricultural composition, an insect control composition (e.g. insecticide composition), or a plant protection composition.

In any of these embodiments, the compound or combination of compounds of the invention may be provided as part of a composition, such as an agricultural composition, an insect control composition (e.g. insecticide composition) or a plant protection composition. Reference to application or use of a compound or combination herein should therefore be construed as encompassing application or use of a suitable composition thereof, unless the context demands otherwise.

The composition typically comprises a compound as described herein in admixture with one or more ancillary component such as solvents, carriers, diluents, adjuvants, preservatives, dispersants, emulsifying agents, or synergists.

The composition may further comprise a combination with one or more additional active insecticides as described herein.

The invention further provides a composition, e.g. an agricultural composition, an insect control composition or plant protection composition, comprising a compound of the invention, or a combination thereof, in admixture with one or more solvents, carriers, diluents, adjuvants, preservatives, dispersants, emulsifying agents, or synergists. The composition may be an aqueous composition. Further details of which are explained hereinbelow.

“Agricultural”, as used herein, means suitable for use in the agricultural or agrochemical industry, including horticulture, floriculture and home and garden uses, but also products intended for non-crop related uses such as public health/pest control operator uses to control undesirable insects and rodents, household uses, such as household fungicides and insecticides and agents, for protecting plants or parts of plants, crops, bulbs, tubers, fruits (e.g. from harmful organisms, diseases or pests); for controlling, preferably promoting or increasing, the growth of plants; and/or for promoting the yield of plants, crops or the parts of plants that are harvested (e.g. its fruits, flowers, seeds etc.). Examples of such substances will be clear to the skilled person and preferably in the context of the present invention, include compounds that are active as insecticides (e.g. contact insecticides or systemic insecticides, including insecticides for household use). Other such agrochemicals may be pesticides, growth regulators, nutrients/fertilizers, repellants, defoliants etc.

“Agricultural use”, as used herein, not only includes the use of the insecticidal compounds of the invention, combinations thereof or compositions, and optionally the agrochemicals as defined above (for example, pesticides, growth regulators, nutrients/fertilizers, repellants, defoliants etc.), that are suitable and/or intended for use in field grown crops (e.g., agriculture), but also includes the use of compounds of the invention combinations thereof or compositions and said agrochemicals as defined above that are meant for use in greenhouse grown crops (e.g. horticulture/floriculture) or hydroponic culture systems and even the use of compounds combinations thereof or compositions of the invention and agrochemicals as defined above that are suitable and/or intended for non-crop uses such as uses in private gardens, household uses (for example, herbicides or insecticides for household use), or uses by pest control operators (for example, weed control etc.).

Suitably an insect control composition according to the invention is for controlling insect populations. Formulations of such compositions for controlling insect populations are known to the person skilled in the art and include, but are not limited to liquid emulsifiable concentrates, wettable powders, solutions, suspension concentrates, emulsions, suspoemulsions, granules and water dispersible granules (Mulqueen, 2003). Preferably, said insect control compositions according to the invention comprise an insecticidal compound of the invention, and optionally a combination of further insecticidal compounds. Suitably said insecticidal compound is comprised in a carrier as described hereinbelow.

Compositions of the invention may comprise a pesticide formulation or an agrochemical formulation. A “pesticide formulation” as used herein means any composition comprising a compound or combination of compounds intended for preventing, destroying, repelling, attracting or mitigating any pest. An “agrochemical formulation” as used herein means a composition for agricultural use, comprising a biologically active agent, optionally with one or more additives favoring optimal dispersion, atomization, distribution, retention and/or activity of agrochemicals. As a non-limiting example such additives are diluents, solvents, adjuvants, surfactants, wetting agents, spreading agents, oils, stickers, penetrants, buffering agents, acidifiers, defoaming agents or drift control agents.

A composition as used herein means a composition comprising at least one active substance, suitably an insecticidal compound of the invention, optionally with one or more additives favouring optimal dispersion, atomization, deposition, leaf wetting, distribution, retention and/or uptake of said active substance. It will become clear from the further description herein that a composition as used herein includes biological insect control agents or biological insecticides, and these terms will be interchangeably used in the present application. Accordingly, a composition as used herein includes compositions comprising at least one biological molecule as an active ingredient, substance or principle for controlling pests in plants or in other agro-related settings (such for example in soil). Suitably wherein the at least one biological molecule comprises an insecticidal compound of the invention or a combination thereof. Suitably wherein the pest is an insect. As a non-limiting example, the additives in the compositions disclosed herein may include but are not limited to diluents, solvents, adjuvants, surfactants, wetting agents, spreading agents, oils, stickers, thickeners, penetrants, buffering agents, acidifiers, antisettling agents, anti-freeze agents, photo-protectors, defoaming agents, biocides and/or drift control agents.

Suitably, the compositions of the invention are aqueous compositions.

The compound content of the composition can vary within wide limits. The compound concentration of the composition is suitably an effective amount. The compound concentration of the composition can be from 0.0000001 to 95% by weight of the compound, preferably between 0.0001 and 1% by weight. The terms “effective amount” and “effective dose”, as used herein, mean the amount needed to achieve the desired result or results.

In a specific embodiment the concentration of the compound of the invention contained in the composition may be at least 0.0001% by weight. In a specific embodiment the concentration of the compound of the invention contained in the composition may be up to 50% by weight. In a specific embodiment the concentration of the compound of the invention contained in the composition may be from 0.0001% to 50% by weight. In particular embodiments, the present invention provides compositions comprising at least one insecticidal compound of the invention, wherein the concentration of the at least one compound of the invention in the composition ranges from 0.001% to 50% by weight. In yet another specific embodiment the concentration of the at least one compound of the invention contained in the composition may be from 0.001% to 50% by weight. In yet another specific embodiment the concentration of the at least one compound of the invention contained in the composition may be from 0.01% to 50% by weight. In yet another specific embodiment the concentration of the at least one compound of the invention contained in the composition may be from 0.1% to 50% by weight.

The compositions of the invention, or for use in accordance with the invention, may comprise more than one compound of the invention in combination, and/or with other insecticidal compounds as described herein in combination. Therefore, the compositions of the invention may comprise a first compound of the invention and a second compound of the invention, or a first compound of the invention and a second insecticidal compound, for example. Suitably the first and second compound may be any of those described herein, and may be present in the composition in any relative proportion.

The composition may be an aqueous composition, e.g. a saline composition. The aqueous composition may contain one or more buffers, such as a phosphate buffer (e.g. phosphate buffered saline) or a Tris buffer. Alternatively, the composition may be an oil dispersion or an emulsion, e.g. an oil and water emulsion. Alternatively, the composition may be a suspension, powder, foam, paste, granule, aerosol, impregnated natural and synthetic substance, or encapsulated in polymeric substance for example. A suitable form of the composition may be chosen for the intended use having regard to the target insect, and to its habitat.

Adjuvants may enhance product performance, for example, by increasing the efficiency of the delivery of active ingredients, reducing the level of active ingredient required, or extending the spectrum of effectiveness.

Different types of adjuvants offer various benefits and advantages, which are achieved by modulating properties such as spray formation, spray retention, wetting, deposit formation or uptake.

Adjuvants modulating spray formation may influence spray quality by reducing spray drift and wastage, allowing more of the product to reach the target. This can reduce use rates, leading to a better environmental profile and a potentially more cost-effective solution. Such adjuvants include non-ionic surfactants and emulsifier blends.

Adjuvants modulating spray retention may dissipate the kinetic energy of the droplet during impact, meaning the likelihood of bounce or run-off is reduced. Such adjuvants include alkyl polyglucosides, alkoxylated alcohols, and polyoxyethylene monobranched alcohols (e.g. polyoxyethylene (8) monobranched alcohol).

Adjuvants modulating wetting properties (i.e. wetting agents) may reduce surface tension and contact angle, leading to enhanced coverage. Such adjuvants include polyoxyethylene sorbitan monolaurate (e.g. polyoxyethylene (8) sorbitan monolaurate), surfactant blends, and alkyl polyglucosides.

Adjuvants modulating deposit formation may influence evaporation of water from the droplet and thus provide a more homogeneous distribution. Such adjuvants include alkoxylated polyol esters, polyoxyethylene sorbitan monolaurate (e.g. polyoxyethylene (12) sorbitan monolaurate), and alkyl polyglucoside.

Adjuvants modulating uptake can improve penetration and uptake of active ingredients. e.g. through the insect cuticle, resulting in increased bioavailability. Such adjuvants include alkoxylated polyol esters and polyoxyethylene sorbitan monolaurate (e.g. polyoxyethylene (12) sorbitan monolaurate and polyoxyethylene (16) sorbitan monolaurate).

Dispersants may be aqueous or non-aqueous. An oil dispersion (OD) formulation typically comprises a solid active ingredient dispersed in oil. The oil can vary from paraffinic to aromatic solvent types and vegetable oil or methylated seed oils. Typically, the active ingredient is uniformly suspended in the oil phase. Although primarily used for water sensitive active ingredients, OD formulations have extended to other active ingredients due to their better spray retention, spreading, foliar uptake, and penetration enhancement (e.g. across the insect cuticle) as the carrier oil often acts as an adjuvant.

Oils suitable for use in OD dispersions include linseed, rapeseed and soyabean oils.

Aqueous dispersants may be used, for example, to improve stability in the spray tank after dilution in water, and may include modified styrene acrylic polymers, and polymeric amphoteric dispersants and adjuvants.

An emulsifier may be employed to emulsify a continuous oil phase into water when an OD formulation is diluted prior to being sprayed. The emulsifier may be selected based upon its ability to spontaneously form the emulsion. Their performance is primarily dictated by the nature of the surfactant and their collective effect on how they arrange themselves at the oil/water interface. Examples include polyoxyethylene sorbitol hexaoleate (e.g. polyoxyethylene (40) sorbitol hexaoleate), emulsifier blends, and calcium alkylaryl sulphonate.

The composition may further comprise an adhesive or a dye.

The compound may be provided in the form of a concentrate, for dilution prior to application. Alternatively, the compound may be provided in a solid form to be suspended or dissolved prior to formulation.

The compositions as disclosed herein are themselves in fairly diverse, solid or liquid, forms. As solid composition forms, there may be mentioned dustable powders (content of active substance which may be up to 100%) and granules, in particular those obtained by extrusion, by compacting, by impregnation of a granulated carrier, by granulation using a powder as starting material (the content of active substance in these granules being between 0.5 and 80% for these latter cases). Such solid compositions may be optionally used in the form of a liquid which is viscous to a greater or lesser degree, depending on the type of application desired, for example by diluting in water. As liquid composition forms or forms intended to constitute liquid compositions during application, there may be mentioned solutions, in particular water-soluble concentrates, emulsions, suspension concentrates, wettable powders (or spraying powder), oils and waxes. The suspension concentrates, which can be applied by spraying, are prepared so as to obtain a stable fluid product which does not form a deposit and they usually contain from 10 to 75% of active substance, from 0.5 to 15% of surfactants, from 0.1 to 10% of thixotropic agents, from 0 to 10% of appropriate additives, such as antifoams, corrosion inhibitors, stabilizers, penetrating agents and adhesives and, as carrier, water or an organic liquid in which the active substance is not or not very soluble: some organic solids or inorganic salts may be dissolved in the carrier to help prevent sedimentation or as antigels for water.

A “carrier”, as used herein, means any solid, semi-solid or liquid carrier in or on(to) which an active substance, such as the insecticidal compounds or combinations of the invention, can be suitably incorporated, included, immobilized, adsorbed, absorbed, bound, encapsulated, embedded, attached, or comprised. Non-limiting examples of such carriers include nanocapsules, microcapsules, nanospheres, microspheres, nanoparticles, microparticles, liposomes, vesicles, beads, a gel, weak ionic resin particles, liposomes, cochleate delivery vehicles, small granules, granulates, nano-tubes, bucky-balls, water droplets that are part of an water-in-oil emulsion, oil droplets that are part of an oil-in-water emulsion, organic materials such as cork, wood or other plant-derived materials (e.g. in the form of seed shells, wood chips, pulp, spheres, beads, sheets or any other suitable form), paper or cardboard, inorganic materials such as talc, clay, microcrystalline cellulose, silica, alumina, silicates and zeolites, or even microbial cells (such as yeast cells) or suitable fractions or fragments thereof.

In one embodiment, the carrier is a liposome. In one embodiment, the composition of the invention comprises one more liposomes suitably wherein each liposome comprises a compound of the invention, or combination thereof. Suitably therefore the invention provides a composition or formulation comprising a plurality of liposomes, said liposomes comprising a compound of the invention, or a combination thereof. Suitably the liposomes comprise a lipid component, suitably which may be soy lecithin and glycerol. Suitably the liposomes are formed of soy lecithin and glycerol. Suitably the liposomes are formed of 25 mg/ml of glycerol and 3% glycerol. Suitably the liposomes comprise an effective concentration of the compound of the invention. Suitably the liposomes comprise around 10⁻⁴ M of a compound of the invention, or a combination thereof. Suitably therefore the invention provides a composition or formulation comprising a plurality of liposomes, said liposomes comprising a compound of the invention, or a combination thereof, soy lecithin (preferably 25 mg/ml) and glycerol (preferably 3%).

Suitably the liposomes may be manufactured by the Mozafari Heating Method (Mozafari, M. R. “Nanoliposomes: preparation and analysis.” Liposomes: Methods and Protocols, Volume 1: Pharmaceutical Nanocarriers (2010): 29-50. Suitably such a method comprises (a) admixing the compound of the invention with glycerol, suitably with a 50% glycerol solution, (b) admixing the mixture of step (a) with soy lecithin, suitably with 350 mg of soy lecithin in an aqueous solution, (c) heating the mixture of step (b) whilst stirring to form liposomes, suitably at around 60° C. and at around 800 RPM for around 40 minutes, (d) annealing the liposomes, suitably by placing in 40 C water for around 1-2 hours, and (e) sonicating the liposomes, suitably for around 30 minutes in a sonicator bath.

Suitably the liposomes have an average diameter of around 150 to 250 nm, suitably around 175 nm to 225 nm, suitably around 190 nm to 210 nm, suitably around 200 nm. Suitably the size of the liposomes is determined by using Dynamic light scattering (DLS), for example using a Malvern Zetasizer Nano. Suitably the liposomes have a polydispersity index of between 0.25 to 0.35, suitably 0.26 to 0.33, suitably 0.26 to 0.31, suitably 0.26 to 0.30, suitably 0.26 to 0.29, suitably 0.26 to 0.28, suitably around 0.26.

In the compositions of the invention, the carrier with the one or more insecticidal compounds may for example be maintained as a wettable powder, wettable granule, emulsifiable concentrate, suspension concentrate, microemulsion, capsule suspension, dry microcapsule, tablet or gel or be suspended, dispersed, emulsified or otherwise brought in a suitable liquid medium (such as water or another suitable aqueous, organic or oily medium) so as to provide a (concentrated) liquid composition of the invention that has a stability that allows the composition of the invention to be suitably stored or (where necessary after further dilution) applied to the intended site of action. Suitably the composition of the invention can be transported and/or stored prior to final use, optionally (and usually preferably) as a suitable liquid concentrate, dry powder, tablet, capsule suspension, slurry or “wet cake”, which can be suitably diluted, dispersed, suspended, emulsified or otherwise suitably reconstituted by the end user prior to final use. The composition of the invention allows to be applied to the intended site of action using any suitable or desired manual or mechanical technique such as spraying, pouring, dripping, brushing, coating, dripcoating, applying as small droplets, a mist or an aerosol or any other suitable technique. In one embodiment, said intended site of action is an intact living insect, even more preferably an insect surface.

Commercially available base formulations may also be suitable for use in formulating the compounds described in this specification, such as Armid® FMPC (Akzo Nobel).

The composition may comprise one or more synergists, i.e. compounds which increase the efficacy of insecticides against their targets, often by inhibiting an insect's ability to metabolise the active agent. Common synergists include piperonyl butoxide and MGK-264 (n-octyl bicycloheptane dicarboximide), or peptidase inhibitors.

The composition may comprise one or more agents which promote stability of the insecticidal compounds of the invention. Suitably the one or more agents which promote stability may prevent degradation of the insecticidal compounds of the invention. Suitable agents which prevent degradation of the insecticidal compounds of the invention may inhibit or reduce the activity of enzymes, suitably of enzymes which act to degrade proteins, suitably proteases, for example. Suitably therefore the composition may comprise one or more protease inhibitors, suitably which may be selected from: Bowman-Birk Inhibitors, Kunitz Inhibitors, Cystatin, Trypsin Inhibitors, Serpins, Tannins, Proteinase Inhibitor-II (PI-II), and Alpha-All.

The composition may further comprise one or more additional, attractants, sterilizing agents, acaricides, nematicides, fungicides, growth-regulating substances or herbicides.

In some embodiments, the compositions may comprise one or more further agricultural chemicals or agrochemicals such as herbicides (e.g. contact herbicides or systemic herbicides, including herbicides for household use), fungicides (e.g. contact fungicides or systemic fungicides, including fungicides for household use), nematicides (e.g. contact nematicides or systemic nematicides, including nematicides for household use) and other pesticides or biocides (for example agents for killing insects or snails); as well as fertilizers; growth regulators such as plant hormones; micro-nutrients, safeners, pheromones; repellants; insect baits; and/or active principles that are used to modulate (i.e. increase, decrease, inhibit, enhance and/or trigger) gene expression (and/or other biological or biochemical processes) in or by the targeted plant (e.g. the plant to be protected or the plant to be controlled), such as nucleic acids (e.g., single stranded or double stranded RNA, as for example used in the context of RNAi technology) and other factors, proteins, chemicals, etc. known per se for this purpose, etc.

Examples of such agrochemicals will be clear to the skilled person; and for example include, without limitation: glyphosate, paraquat, metolachlor, acetochlor, mesotrione, 2,4-D, atrazine, glufosinate, sulfosate, fenoxaprop, pendimethalin, picloram, trifluralin, bromoxynil, clodinafop, fluroxypyr, nicosulfuron, bensulfuron, imazetapyr, dicamba, imidacloprid, thiamethoxam, fipronil, chlorpyrifos, deltamethrin, lambda-cyhalotrin, endosulfan, methamidophos, carbofuran, clothianidin, cypermethrin, abamectin, diflufenican, spinosad, indoxacarb, bifenthrin, tefluthrin, azoxystrobin, thiamethoxam, tebuconazole, mancozeb, cyazofamid, fluazinam, pyraclostrobin, epoxiconazole, chlorothalonil, copper fungicides, trifloxystrobin, prothioconazole, difenoconazole, carbendazim, propiconazole, thiophanate, sulphur, boscalid and other known agrochemicals or any suitable combination(s) thereof.

The composition may be a bait composition for ingestion by the target insect. A bait composition may comprise one or more phagostimulants, i.e. a substance which will entice the insect to ingest the compound. Phagostimulants may include artificial sweeteners, amino acids, other peptides or proteins and carbohydrates (e.g. glucose, fructose, sucrose, maltose) etc. Examples include honey, syrups and aqueous solutions of sucrose.

Commercially available base formulations may also be suitable for use in formulating the compounds described in this specification, such as Armid® FMPC (Akzo Nobel).

The composition may comprise one or more synergists, i.e. compounds which increase the efficacy of insecticides against their targets, often by inhibiting an insect's ability to metabolise the active agent. Common synergists include piperonyl butoxide and MGK-264 (n-octyl bicycloheptane dicarboximide), or peptidase inhibitors.

There is also provided a combination comprising a compound of the invention, or a salt or solvate thereof, in combination with one or more additional active insecticides. The additional active insecticide may be selected from pyrethrins or pyrethroids, or other peptide analogues. The insecticides may also include, for example, phosphates, carbamates, carboxylates, chlorinated hydrocarbons, phenylureas and substances produced by microorganisms. The additional active insecticide may be a further kinin analogue, an AKH peptide, a DH31 peptide, DH44 peptide, a pyrokinin peptide or a CAPA peptide (e.g. a CAPA-1, CAPA-2 or CAPA-3 analogue).

The composition may further comprise one or more additional active insecticides. The compositions of the invention, or for use in accordance with the invention, may comprise a compound of the invention in combination with a further kinin analogue, an AKH peptide, a DH31 peptide, DH44 peptide, a pyrokinin peptide or a CAPA peptide (e.g. a CAPA-1, CAPA-2 or CAPA-3 analogue).

### Methods of Producing the Insecticidal Compounds

The insecticidal compounds described herein may be produced by any method known in the art for the production of peptides. In one embodiment, the insecticidal compounds may be produced by a chemical method, suitably a chemical synthesis method.

Any suitable chemical method may be used to synthesis the insecticidal compounds of the invention.

Suitably the insecticidal compounds are peptides. Suitably therefore they are synthesised by solid-state peptide synthesis. Suitably such synthesis may be carried out by using commercially available machines such as the Biotage Initiator+Alstra microwave-assisted peptide synthesiser or the CEM Liberty Prime microwave-assisted peptide synthesiser.

In a further aspect of the invention, there is provided an isolated insecticidal compound produced by a process of the invention. Suitably an isolated insecticidal compound of formula (I) or (XX) produced by a process of the invention.

In a further aspect of the invention there is provided a method of producing a composition as disclosed herein, at least comprising the steps of: (a) obtaining at least one insecticidal compound of formula (I) or (XX); and (b) formulating the insecticidal compound into a composition.

In a further aspect of the invention there is provided a method of producing and/or manufacturing a variant of the insecticidal compound of formula (I). Such may comprise the steps of: (i) modifying the peptide Z of formula (I) or (XX) by adding, replacing or deleting at least one amino acid; (ii) assessing the so created variant for its insecticidal activity and—optionally—at least one property selected from the group consisting of biostability, chemical stability, bioavailability, solubility (incl. the ability to form stable formulations), producibility, and production costs. Where the insecticidal activity is reduced in comparison to the insecticidal compound comprising the unmodified peptide Z, repeating the process of steps (i) and (ii) until a variant with improved insecticidal activity is obtained. Where the insecticidal activity is improved in comparison to insecticidal compound comprising the unmodified peptide Z, the method may include further septs of manufacturing, isolating and purifying the variant. Screening for insecticidal activity and the other above-described properties can be conducted as described below or generally known to the person skilled in the art.

Suitably the step of obtaining at least one insecticidal compound comprises (a) chemically synthesising the insecticidal compound.

Suitable compositions are described hereinabove. Suitable manufacturing methods for formulating the composition are known in the art and include, but are not limited to, high or low shear mixing, wet or dry milling, drip-casting, encapsulating, emulsifying, coating, encrusting, pilling, extrusion granulation, fluid bed granulation, co-extrusion, spray drying, spray chilling, atomization, addition or condensation polymerization, interfacial polymerization, in situ polymerization, coacervation, spray encapsulation, cooling melted dispersions, solvent evaporation, phase separation, solvent extraction, sol-gel polymerization, fluid bed coating, pan coating, melting, passive or active absorption or adsorption.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

The invention includes the combination of the aspects and preferred features described except where such a combination is impermissible or expressly avoided.

### BRIEF DESCRIPTION OF THE FIGURES

Aspects and embodiments of the present invention will now be discussed with reference to the accompanying figures. Further aspects and embodiments will be apparent to those skilled in the art. All documents mentioned in this text are incorporated herein by reference.

FIG. 1 shows activity data of unformulated peptides according to the present invention against D. suzukii FIG. 2 shows activity data of unformulated peptides according to the present invention against M. persicae.

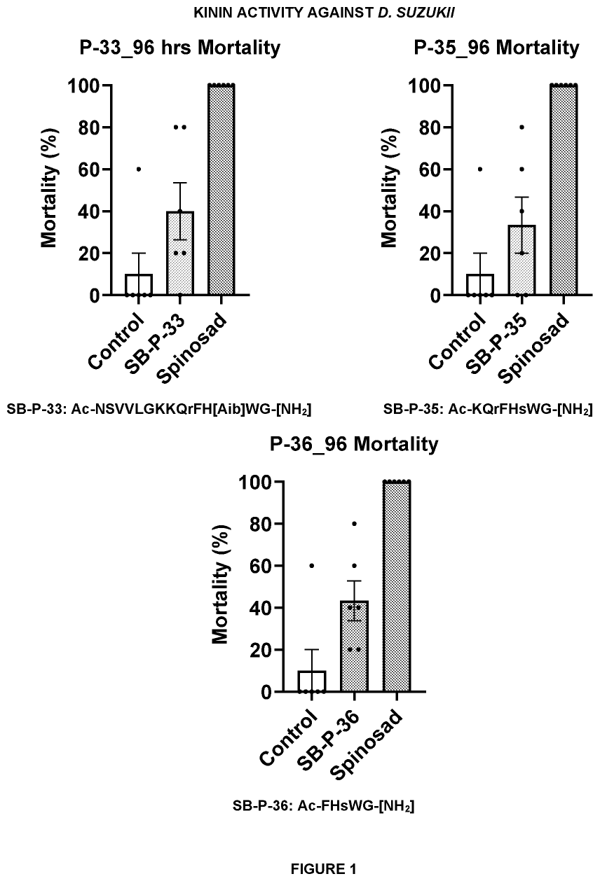

FIGS. 3-5 shows activity data of unformulated peptides according to the present invention, in combination with one or more other peptides, against M. persicae.

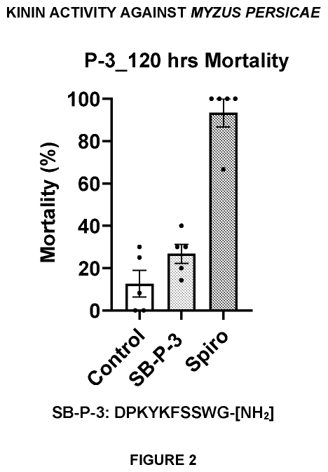

FIG. 6 shows activity data of unformulated peptides according to the present invention against Plutella xylostella

FIG. 7 shows Scheme 1: Synthetic route taken toward SB-P-33. A general overview of the workflow involved is given in the inset. Positions in which double coupling steps were used are indicated by asterisks.

FIG. 8 shows Scheme 2: Synthetic route taken toward SB-P-35. A general overview of the workflow involved is given in the inset.

FIG. 9 shows Scheme 3: Synthetic route taken toward SB-P-36. A general overview of the workflow involved is given in the inset.

FIG. 10 shows Scheme 5: Synthetic route taken toward SB-P-54. An overview of the typical workflow is given in the inset.

FIG. 11 shows the café assay experimental set up.

FIG. 12 shows the leaf dip assay experimental set up.

FIG. 13 shows annotated LC-MS traces of (top to bottom): SB-P-70 in Schneider's medium (a) and SB-P-70 after 24 h tissue incubation (b).

FIG. 14 shows annotated LC-MS traces of SB-P-103 in Schneider's medium (a) and SB-P-103 after 24 h tissue incubation (b).

FIG. 15 shows peptide integrity of SB-P-70 and SB-P-103 after incubating with insect tissues for 24 hours.

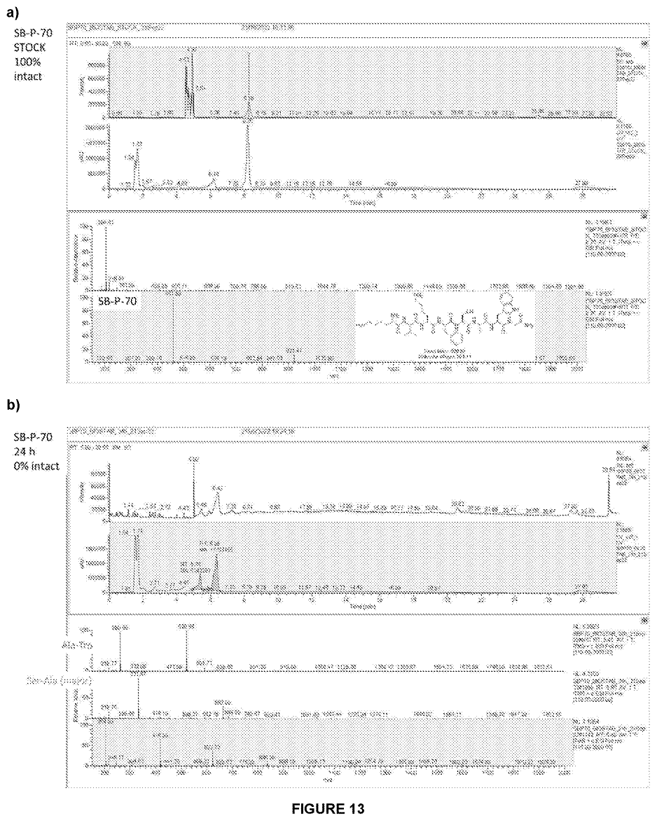

FIG. 16 shows bee oral toxicity results represented as % survival after 96 hours of application of SB-P-32, SB-P-065, SB-P-066, and a combination of SB-P-33 and SB-P-51 (pyrokinin).

FIG. 17 shows the chemical synthesis route for synthesising SB-P-65.

FIG. 18 shows an HPLC trace of SB-P-65.

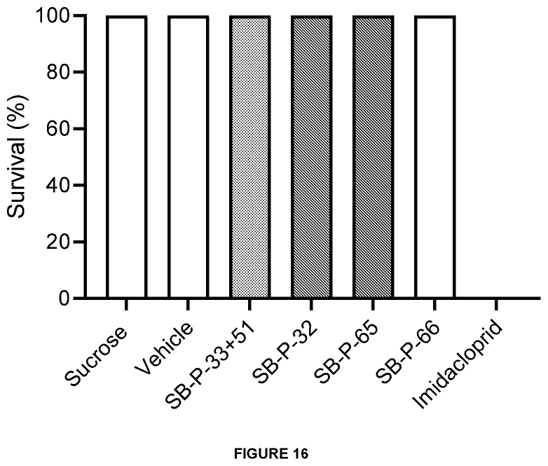

FIG. 19 shows LC-MS traces of SB-P-65.

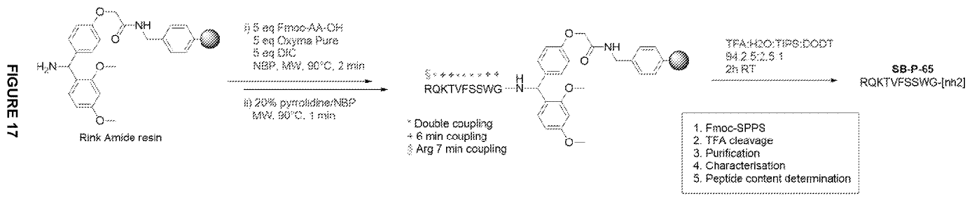

FIG. 20 shows activity data of unformulated SB-P-65 against Myzus persicae after 120 hours.

FIG. 21 shows activity data of unformulated SB-P-65 against Myzus persicae over time.

FIG. 22 shows the chemical synthesis route for synthesising SB-P-66.

FIG. 23 shows an HPLC trace of SB-P-66.

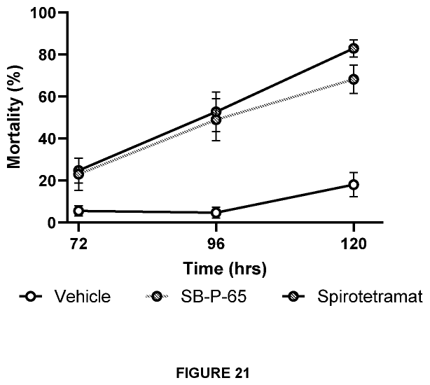

FIG. 24 shows LC-MS traces of SB-P-66.

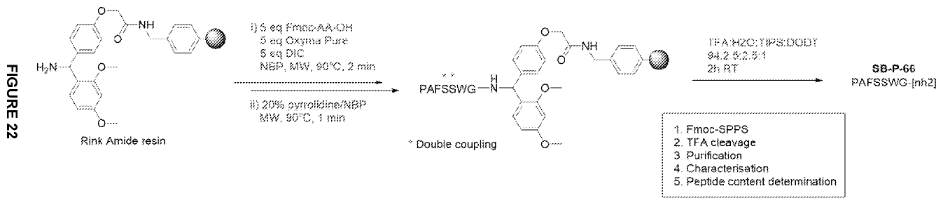

FIG. 25 shows activity data of unformulated SB-P-66 against Myzus persicae after 120 hours.

FIG. 26 shows activity data of unformulated SB-P-66 against Myzus persicae over time.

FIG. 27 shows the chemical synthesis route for synthesising SB-P-70.

FIG. 28 shows an HPLC trace of SB-P-70.

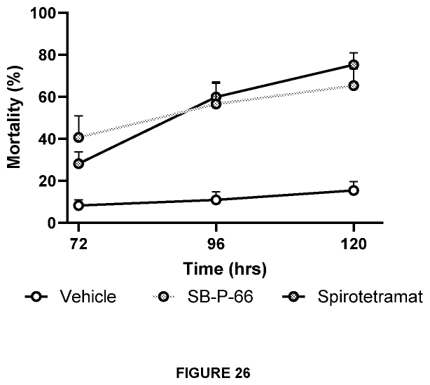

FIG. 29 shows LC-MS traces of SB-P-70.

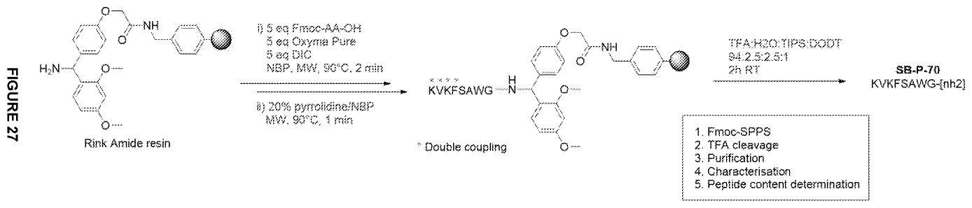

FIG. 30 shows activity data of unformulated SB-P-70 against Plutella xylostella after 96 hours.

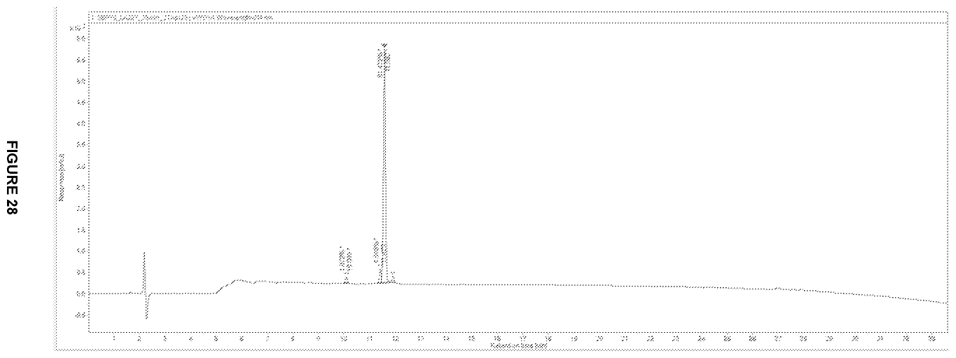

FIG. 31 shows the lepidoptera mortality assay experimental set up.

FIG. 32 shows activity data of unformulated SB-P-70 against Spodoptera frugiperda after 168 hours.

FIG. 33 shows activity data of SB-P-65 against Myzus persicae after 120 hours using commercial Silwet-408 as an adjuvant.

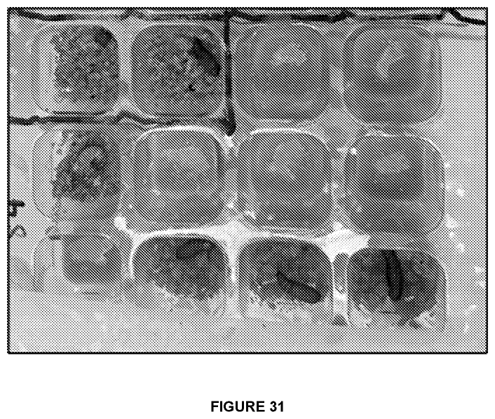

FIG. 34 shows activity data of unformulated SB-P-65 and SB-P-66 against naturally infested Aphis fabae in field trials after 2 days of first application (App. A) and 16 days after second application (App. B) conducted by PGRO on vining peas.

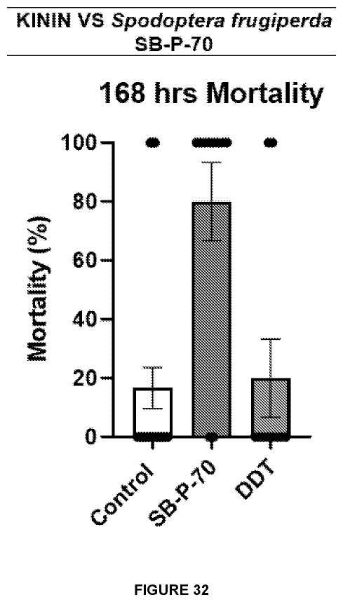

FIG. 35 shows activity data of unformulated SB-P-65 and SB-P-66 against naturally infested Aphis gossypii in field trials after 3 days of application (App. A) and 7 days after application conducted by AgriScience on Cotton.

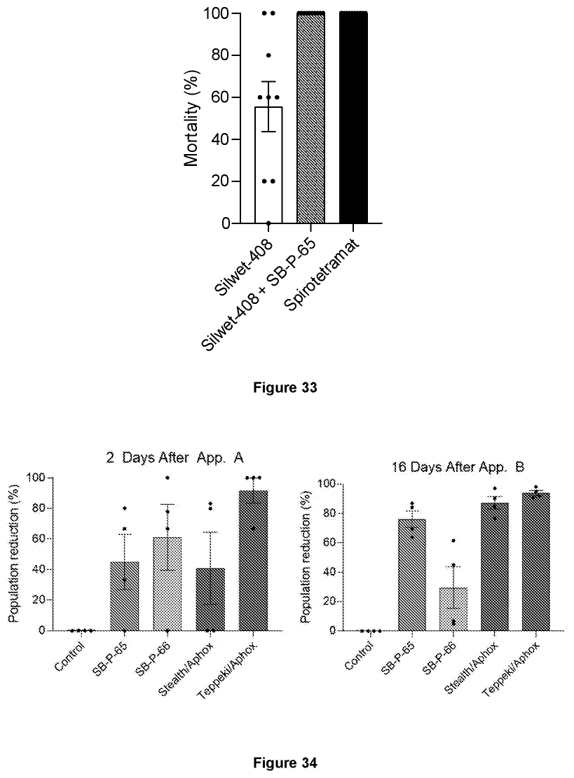

FIG. 36 shows activity data of unformulated SB-P-65 and SB-P-66 against Aphis gossypii in field trials over time conducted by AgriScience on Melon.

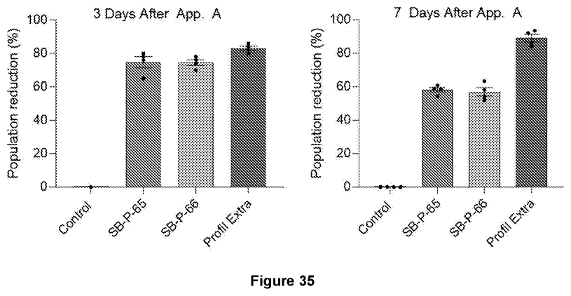

FIG. 37 shows activity data of unformulated SB-P-65 and SB-P-66 against naturally infested Aphis fabae in field trials after 4 days of application and 8 days after application conducted by PGRO on vining peas.

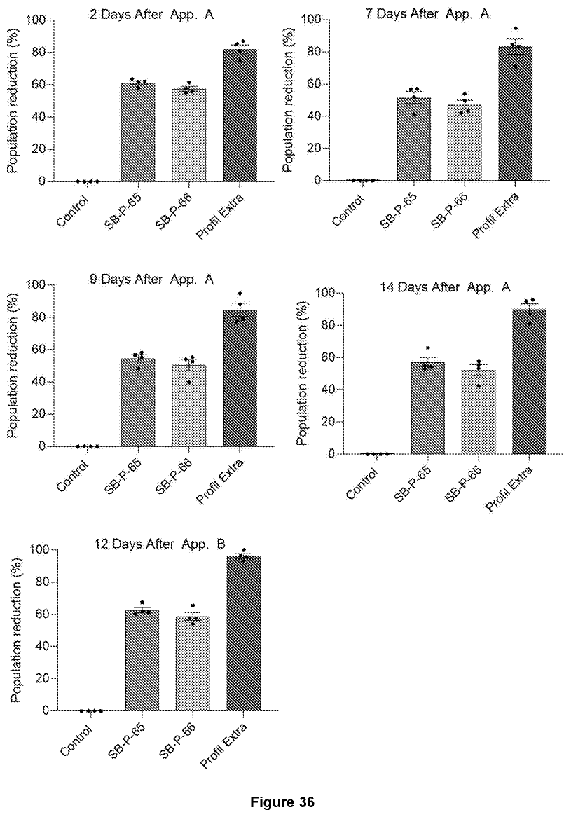

FIG. 38 shows activity data of unformulated SB-P-66 against naturally infested Amrasca biguttula (leaf hopper) in field trials overtime.

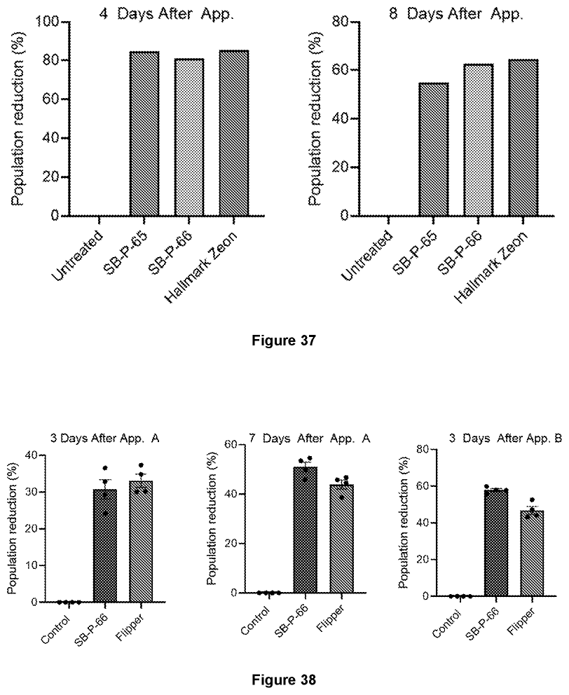

### EXAMPLES

### General Procedures

All amino acids are of L-configuration unless otherwise stated. Standard Fmoc-protected amino acids were purchased from CEM Corporation or Pepceuticals.

Suppliers of specialist amino acids are indicated as and when appropriate, as is peptide synthesis resin supplier. Peptide-grade DMF was purchased from Rathburn.

Peptides were synthesised on a Biotage Initiator+Alstra microwave-assisted peptide synthesiser or a CEM Liberty Prime microwave-assisted peptide synthesiser as specified.

High-resolution mass spectrometry (HRMS) was performed on a Bruker microTOF-Q II (ESI+).

Peptides were purified on a reverse-phase Dionex HPLC system equipped with Dionex P680 pumps and a Dionex UVD170U UV-vis detector (monitoring at 214 nm and 280 nm), using a Phenomenex, Gemini, C18, 5 μm, 250×21.2 mm column. Gradients were performed using solvents consisting of A (H₂O+0.1% TFA) and B (MeCN+0.1% TFA) and fractions were lyophilised on a Christ Alpha 2-4 LO plus freeze dryer.

Pure peptides were analysed on a Shimadzu reverse-phase HPLC (RP-HPLC) system equipped with Shimadzu LC-20AT pumps, a SIL-20A autosampler and a SPD-20A UV-vis detector (monitoring at 214 nm and 280 nm) using a Phenomenex, Aeris, 5 μm, peptide XB—C18, 150×4.6 mm column at a flow rate of 1 mL/min. RP-HPLC gradients were run using a solvent system consisting of solution A (100% H₂O+0.1% TFA) and B (100% MeCN+0.1% TFA). Typically, two gradients were used to characterise each peptide; a gradient from 5% to 95% solution B over 20 min (incorporating a 2 min hold at 5% solution B and a 5 min wash at 95% solution B at the start and end of the gradient respectively) and a gradient from 5-95% solution B over 50 min (incorporating a 5 min hold at 5% solution B and a 5 min wash at 95% solution B at the start and end of the gradient respectively). In some cases, specialised gradients were used and this is indicated where appropriate. Analytical RP-HPLC data is reported as column retention time (tR) in minutes (min). Analytical columns were maintained at ambient temperature.

LC-MS analysis was performed on a Thermo Scientific LCQ Fleet quadropole mass spectrometer of m/z range 50-2000 Da with an ESI source coupled to a Dionex Ultimate 3000 LC. Analyses were performed on a ReprosilGold 120 C18, 3 μm 150×4 mm column using a linear gradient of buffer A (95/5 H₂O/MeCN with 0.1% v/v TFA) to buffer B (95/5 MeCN/H₂O with 0.1% v/v TFA) over 20 min (incorporating a 2 min hold at 0% solution B and a 5 min wash at 100% solution B at the start and end of the gradient respectively). In cases where a specialised analytical RP-HPLC gradient was used to characterise a compound, an analogous LC-MS gradient was used. Analytical RP-HPLC and LC-MS samples were injected as 25 μL of a stock with concentration of 1 mg/mL in H₂O/MeCN with 0.1% v/v TFA. LC-MS column oven temperature was maintained at 30° C.

High-resolution mass spectrometry (HRMS) of pure peptides was performed on a Bruker microTOF-Q II (ESI+).

Proton nuclear magnetic resonance spectra (¹H NMR) for the purpose of peptide content calculations were recorded on an AVANCE III 400 Bruker (400 MHz). Proton chemical shifts are expressed in parts per million (ppm, 5 scale) and are referenced to residual protium in the NMR solvent (CDCl₃, δ 7.26; CD₃OD, δ 3.31 and D₂O, δ 4.79). The following abbreviations were used to describe peak patterns when appropriate: br=broad, s=singlet, d=doublet, t=triplet, q=quadruplet, m=multiplet. Coupling constants, J, are reported in Hertz unit (Hz).

### General Procedure for Automated Peptide Synthesis

### Biotage Initiator+Alstra Synthesiser:

Fmoc-protected amino acids were prepared as a 0.2 M (0.1 mmol syntheses), 0.5 M (0.2 mmol syntheses) or 0.7 M (0.5 mmol syntheses) solution in DMF. 5 equivalents of amino acid (relative to the resin loading) were used during coupling cycles. Oxyma and diisopropyl carbodiimide (DIC) were prepared as 0.2 M (0.1 mmol syntheses), 0.5 M (0.2 mmol syntheses) or 0.7 M (0.5 mmol syntheses) solutions in DMF. 5 equivalents of Oxyma and 5 equivalents of DIC (relative to resin loading) were used during coupling cycles. For Fmoc-deprotections, a solution of 20% morpholine (with 5% formic acid) in DMF was used. Coupling reactions were performed under microwave heating at 90° C. for 2 min with the exception of Fmoc-Cys(Trt)-OH, Fmoc-His(Trt)-OH and Fmoc-Arg(PBf)-OH. Coupling of Fmoc-Cys(Trt) and Fmoc-His(Trt)-OH was performed for 10 min at 50° C. Coupling of Fmoc-Arg(Pbf)-OH was performed for 2 successive cycles (double-coupled) for 2 min at 90° C. Microwave-assisted Fmoc deprotections were carried out at 90° C. for 1 min.

### CEM Liberty Blue Synthesiser

Fmoc-protected amino acids were prepared as a 0.2 M solution in NBP (Tamisolve). 5 equivalents of amino acid (relative to the resin loading) were used during coupling cycles. Oxyma was prepared as a 0.5 M solution in NBP. DIC was prepared as 5 M solution in NBP. 5 equivalents of Oxyma and 5 equivalents of DIC (relative to resin loading) were used during coupling cycles. For Fmoc-deprotections, a solution of 20% pyrrolidine was used. Coupling reactions and Fmoc-deprotections were performed under microwave heating at 90° C. for 2 min and 1 min respectively with the exception of Fmoc-Cys(Trt)-OH, Fmoc-His(Trt)-OH and Fmoc-Arg(Pbf)-OH. Coupling of Fmoc-Cys(Trt) and Fmoc-His(Trt)-OH was performed for 5 min at 50° C. Coupling of Fmoc-Arg(Pbf)-OH was performed for 2 successive cycles of 5 min at 75° C.

### General Procedure for TFA Cleavage of Peptides

Typically, cleavage tests of peptides were performed by taking ~3 mg of dried resin beads and treating them with TFA/TIS/water (95:2.5:2.5:) for 3 h. The filtrate was drained, concentrated and then triturated in cold diethyl ether (Et₂O). The triturate was dissolved in acetonitrile/water, then analysed by RP-HPLC/LC-MS.

Peptides were typically cleaved from the resin in bulk by gently rocking the resin at rt in a cleavage cocktail of TFA/TIS/H₂O (95:2.5:2.5) for 3 h before being drained and the TFA blown off with a steady stream of N₂ gas. Peptides containing cysteine or tryptophan residues were cleaved from the resin using a cleavage cocktail of TFA/TIS/H₂O/DODT (94:2.5:1:2.5) for 3 h. In all cases the crude peptide was triturated with cold Et₂O. Et₂O was removed from the resulting crude peptide pellet under a steady stream of nitrogen. The crude peptide was then redissolved in H₂O/MeCN and purified by RP-HPLC.

Unless otherwise stated herein, peptides were synthesised according to the general procedures above, in line with the exemplary procedures below, or were ordered directly.

### SB-P-33

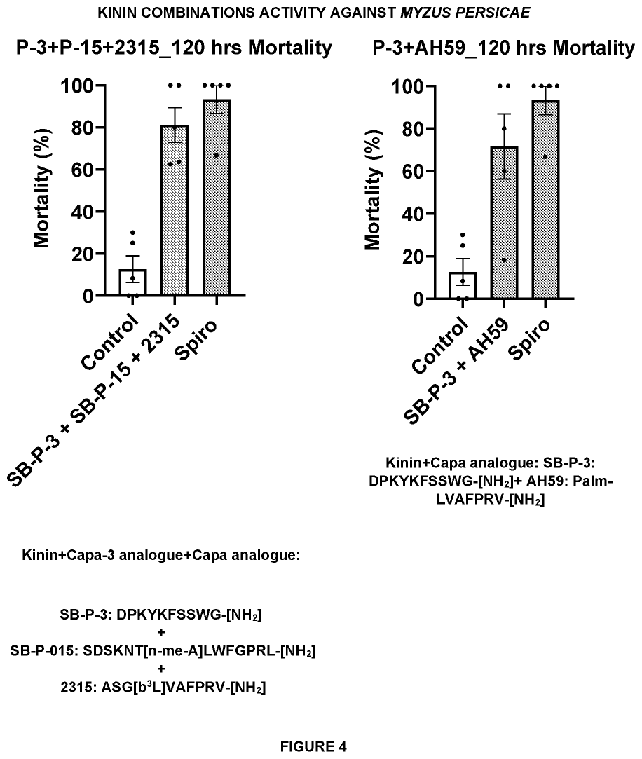

The synthesis of SB-P-33 was performed on the CEM Liberty Prime unit—incorporating double-coupling steps at Val3, Val4, Gln9, D-Arg10, His12 and Aib13 (Scheme 1—FIG. 7).

### SB-P-35:

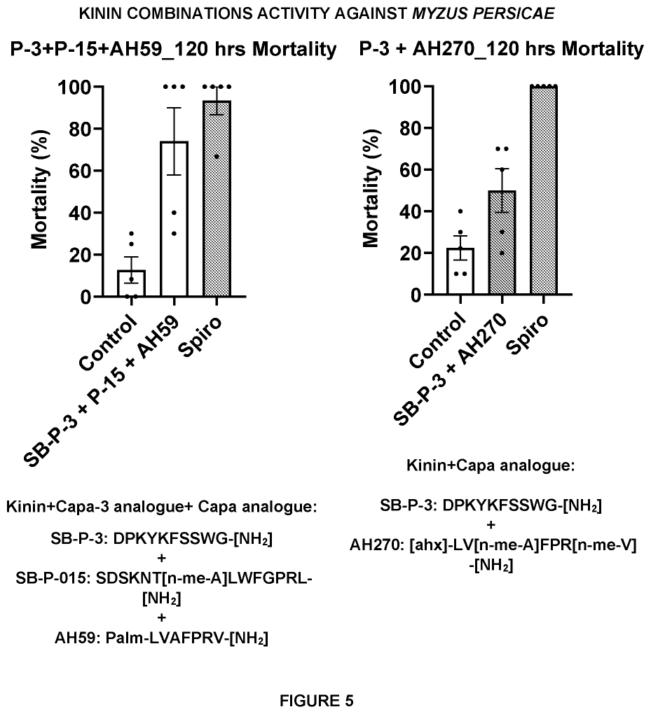

The synthesis of SB-P-35 was performed by Fmoc-SPPS on the CEM Liberty Prime unit. Fmoc-removal was effected using a solution of 25% pyrrolidine in DMF. Coupling reactions were performed using a DIC/Oxyma strategy, in 2 min reactions carried out at 110° C. Acetyl-capping was carried out manually at room temperature using acetic anhydride/DIPEA in DMF (Scheme shown in FIG. 8).

### SB-P-36:

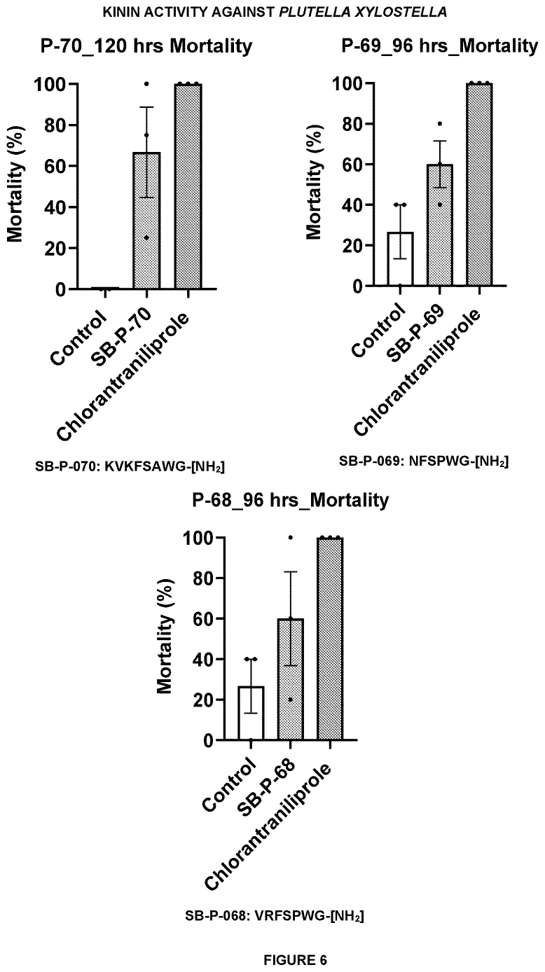

The synthesis of SB-P-36 was performed by Fmoc-SPPS on the CEM Liberty Prime unit. Fmoc-removal was effected using a solution of 25% pyrrolidine in DMF. Coupling reactions were performed using a DIC/Oxyma strategy, in 2 min reactions carried out at 110° C. Acetyl-capping was carried out manually at room temperature using acetic anhydride/DIPEA in DMF (Scheme shown in FIG. 9).

### SB-P-54:

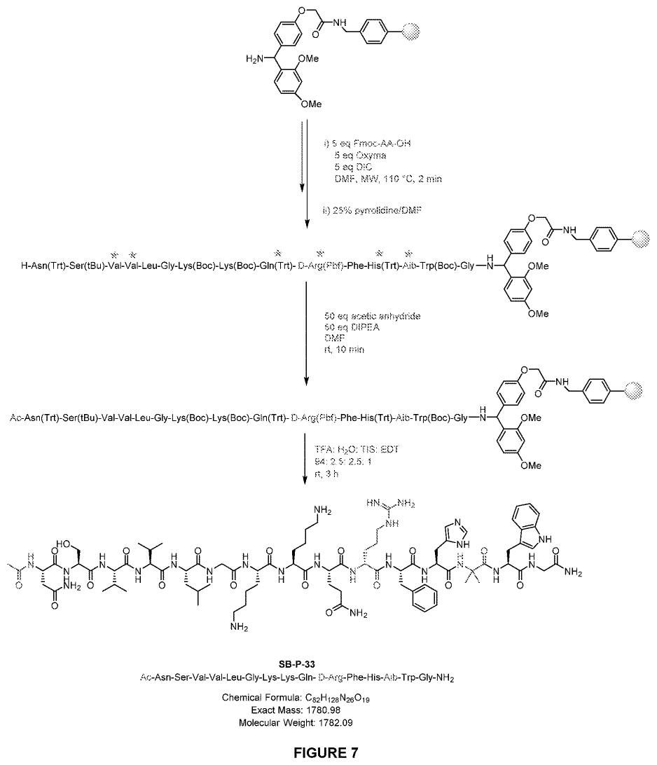

The synthesis of this peptide was performed on Tentagel S RAM resin (f=0.22 mmol/g) by automated Fmoc-SPPS using the Biotage Initiator Alstra peptide synthesiser (Scheme shown in FIG. 10).

Coupling steps were carried out using 4 eq (relative to resin loading) of Fmoc-amino acid (0.2 M) and DIC/Oxyma (0.5 M) for 2 min at 90° C. Fmoc-deprotection was effected in 20% morpholine/DMF (with 5% formic acid additive) for 1 min at 90° C. N-terminal acetyl-capping was achieved by treatment with Ac₂O (50 eq) and DIPEA (50 eq) for 10 min at room temperature.

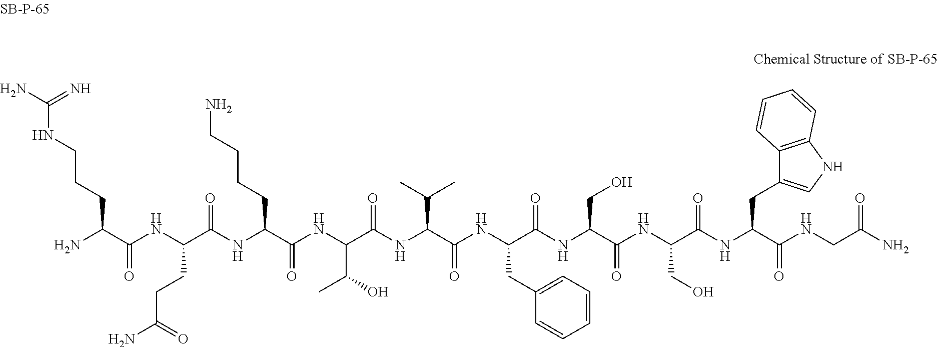 [SMILES: CC(C)[C@H](NC(=O)C(NC(=O)[C@H](CCCCN)NC(=O)[C@H](CCC(N)=O)NC(=O)[C@@H](N)CCCNC(=N)N)[C@@H](C)O)C(=O)N[C@@H](Cc1ccccc1)C(=O)N[C@@H](CO)C(=O)N[C@@H](CO)C(=O)N[C@@H](Cc1c[nH]c2ccccc12)C(=O)NCC(N)=O]

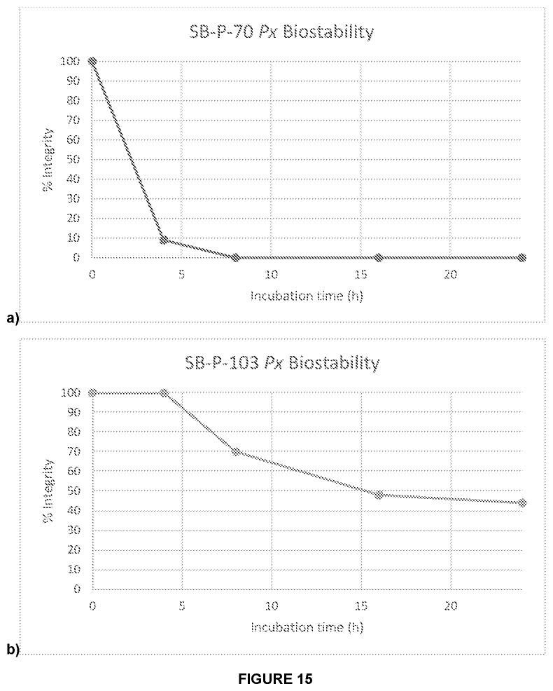

A specific synthesis route for SB-P-65 is shown in FIG. 17. For the synthesis of SB-P-65 at 0.25 mmol, double coupling cycles were used for 2Gln, 5Val, 6Phe and 8Ser. An extended 7 min Arg coupling cycle was used for 1Arg. For the synthesis at 0.5 mmol scale, extended 6 min single couplings were used for 3Lys, 4Thr, 9Trp and 10Gly. Double couplings were used for 2Gln, 5Val, 6Phe, 7Ser and 8Ser. An extended 7 min Arg coupling cycle was used for 1Arg. Purification was performed with a 10 to 20% B gradient monitored at 240 nm, at 60° C. Purity=90%, Yield=41%.

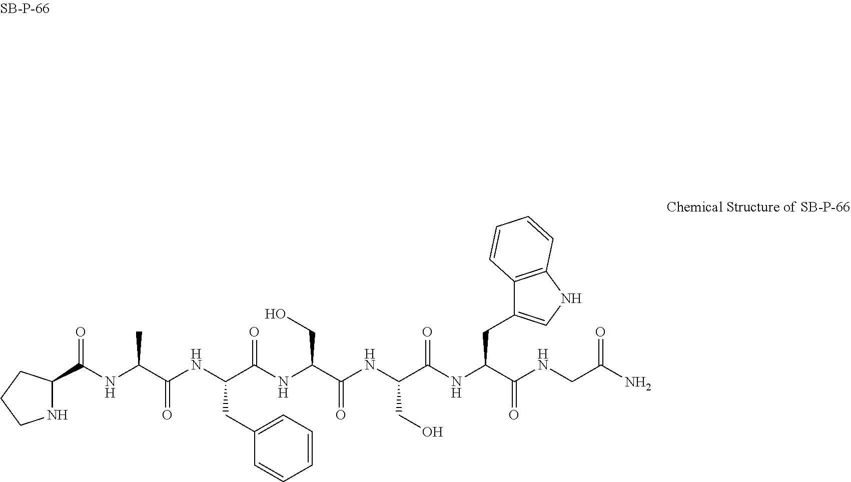 [SMILES: C[C@H](NC(=O)[C@@H]1CCCN1)C(=O)N[C@@H](Cc1ccccc1)C(=O)N[C@@H](CO)C(=O)N[C@@H](CO)C(=O)N[C@@H](Cc1c[nH]c2ccccc12)C(=O)NCC(N)=O]

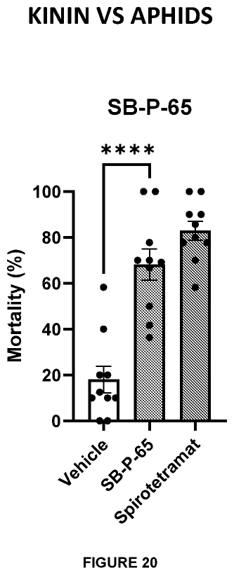

A specific synthesis route for SB-P-66 is shown in FIG. 22. For the synthesis of SB-P-66 at any scale, a double coupling cycle was used for 4Ser and 5Ser. Purification was performed with a 10 to 30% B gradient over 20 min, monitored at 280 nm and at 60° C. Purity=91%, Yield=41%.

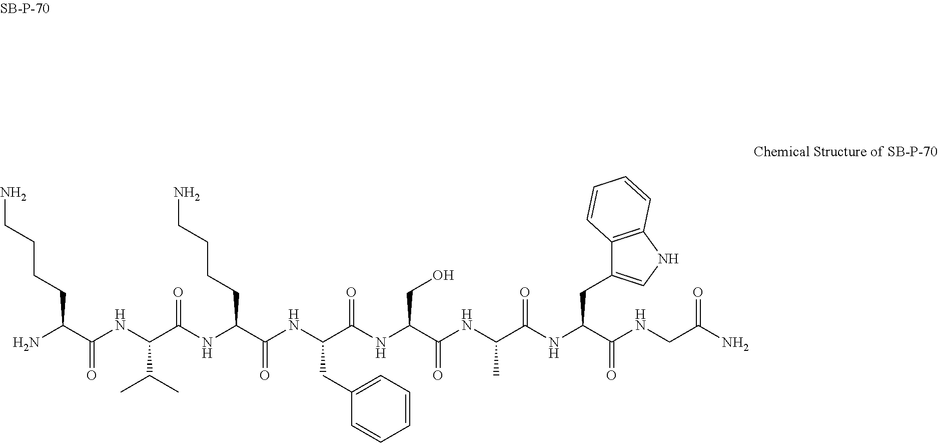 [SMILES: CC(C)[C@H](NC(=O)[C@@H](N)CCCCN)C(=O)N[C@@H](CCCCN)C(=O)N[C@@H](Cc1ccccc1)C(=O)N[C@@H](CO)C(=O)N[C@@H](C)C(=O)N[C@@H](Cc1c[nH]c2ccccc12)C(=O)NCC(N)=O]

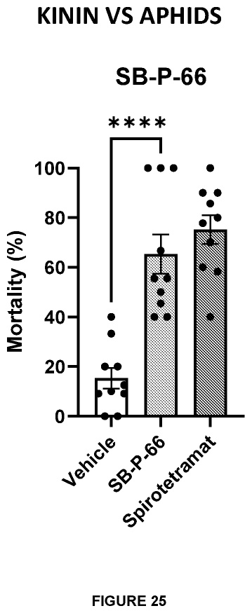

A specific synthesis route for SB-P-70 is shown in FIG. 27. The 0.1 mmol scale synthesis of SB-P-70 was performed using double coupling methods for 1Lys, 2Val, 3Lys and 4Phe. Purification was performed at room temperature, using a gradient of 20 to 80% B over 30 minutes, monitored at 214 nm. Purity=91%, Yield=42%.

### SB-P-101

Synthesis of SB-P-101 was carried out on the CEM Liberty Blue using Rink Amide ProTide LL resin (f=0.18 mmol/g). Fmoc-deprotection reactions were carried out for 1 min at 90° C. in a solution of 20% pyrrolidine in TamiSolve. Coupling reactions were carried out for 2 min at 90° C. using 5 eq Fmoc-AA (relative to resin loading), and 5 eq DIC/Oxyma Pure (relative to resin loading) in TamiSolve. Extended 4 min coupling cycles were used for Fmoc-α-Me-Ser(tBu)-OH and Fmoc-(N-Me)-Phe-OH. A double coupling step was employed for Ser4. The N-terminus of the peptide was acetylated post-synthesis.

Post-synthesis, simultaneous resin-cleavage and side-chain deprotection of the peptide was performed in a cocktail of 94:2.5:2.5:1 (v/v) TFA:TIS:H₂O:DODT for 2 h at room temperature. Following trituration in ice-cold Et₂O, the crude SB-P-101 was purified by semi-preparative RP-HPLC and obtained in >99% purity and 24% overall yield.

### Mortality Assessment Methods

Cafe Assay—D. suzukii

Assay based on—The Capillary Feeder Assay Measures Food Intake in Drosophila melanogaster—PubMed (nih.gov)

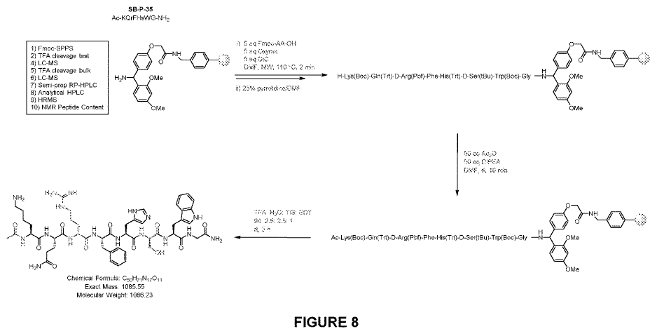

Five same sex Drosophila suzukii are anesthetised via CO₂ and placed into a plastic 2.5 cm×7.5 cm cylindrical vial. The vial is capped by a cotton wool plug which has two holes with one pipette tip placed in each hole to support capillary tubes. Two 5 μl capillary tubes are filled with treatment and weighed on a fine balance before being placed through the pipette tip and into the vial. The vial is placed within an incubator and the flies are given 24 hours to feed. After 24 hrs the capillary tube is removed and weighed and the difference between the start and end is calculated to determine how much food is consumed. A fresh capillary tube (weighed and filled) is placed into the arena, and this is repeated up until the 96 hr point. Mortality (number of dead flies) is counted every 24 hrs. The experimental setup is shown in FIG. 11.

Leaf Dip Assay—Aphids or Plutella xylostella

Based on IRAC susceptibility test method 019 (https://irac-online.org/methods/aphids-adultnymphs/).

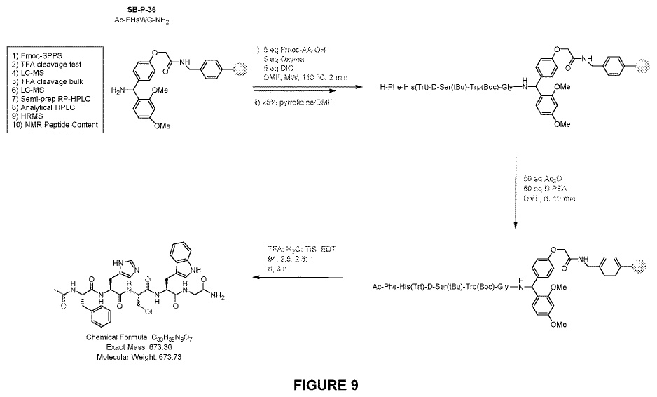

Fill a small 4 cm wide 3 cm high dish with a 1% agar solution (ensuring 1 cm gap between the agar and the lid) and leave to set. Dip a 2.5 cm leaf disc into 3 ml of treatment solution allowing the treatment to coat the leaf. Leave the disc to dry for 1 hour and place onto the set agar. Place insects (aphids or Plutella xylostella) onto each of the leaf discs. Each unit is sealed with a close-fitting, ventilated lid. Place the agar dishes in a small plastic tray lined with moist tissue paper and ensure the water trays in the bottom of the incubator are filled with clean water and place within the incubator. An assessment of mortality is made at 72, 96 and 120 hours. The leaf dip assay set up is shown in FIG. 12.

### Biostability

Plutella xylostella Tissue Biostability Study of SB-P-70 and SB-P-103

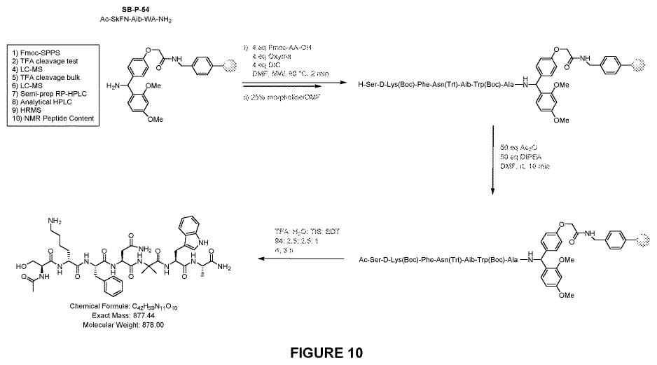

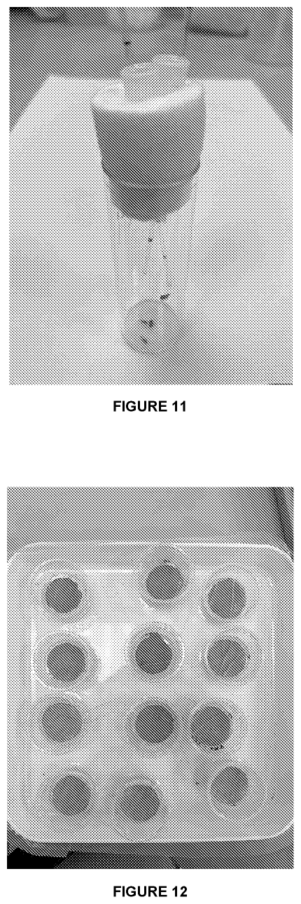

SB-P-70 and SB-P-103 were incubated with the gut and MT tissue and contents of final-stage P. xylostella larvae in Schneider's medium at 25° C. Control samples were prepared with peptides in Schneider's medium with no tissue and tissue in Schneider's medium with no peptides. The condition of the peptides after incubation with the tissue was assessed by LC-MS at: 4 h; 8 h; 16 h; and 24 h time points (FIGS. 13 and 14).

Proteolytic cleavage was identified to have occurred for SB-P-70 primarily between Ser5 and Ala6. At the 24 h time point, the peptide had been completely degraded. In contrast, SB-P-103 remained 44% intact after 24 h tissue incubation.

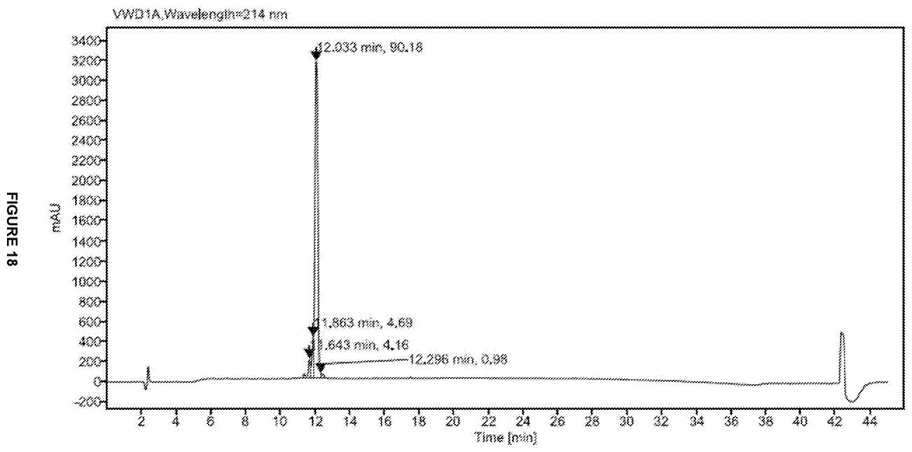

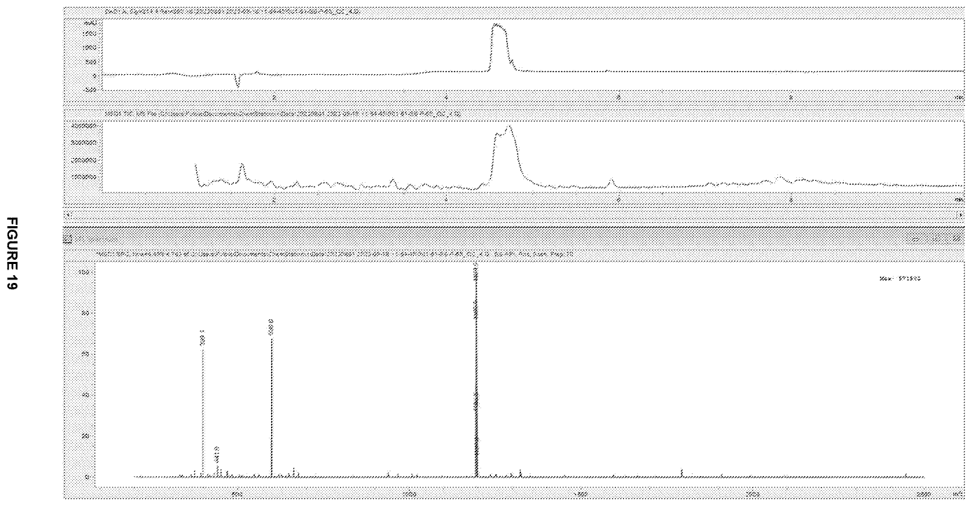

Leaf dip assays for aphids were performed with SB-P-65, and compared to a negative control vehicle and a current commercial insecticide Spirotetramat. The activity of SB-P-65 is shown in FIGS. 20 and 21; SB-P-65 has good activity whilst being eco-friendly and targeted to specific pests unlike broad spectrum chemical insecticides such as Spirotetramat.

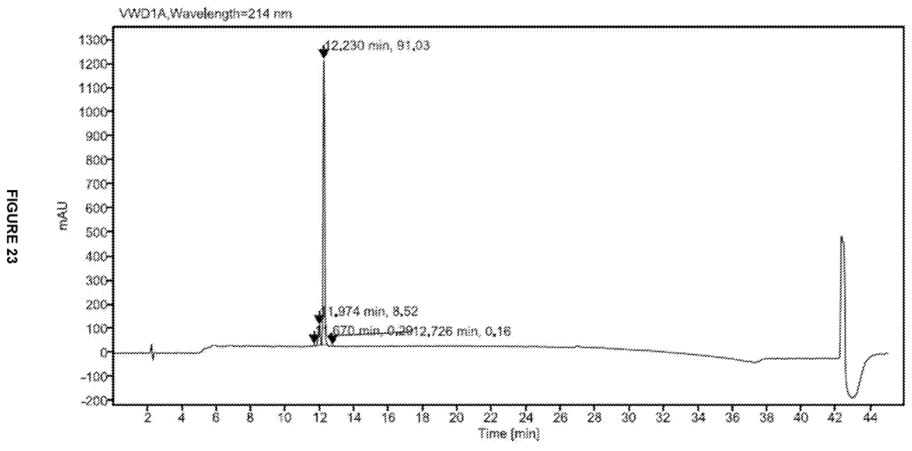

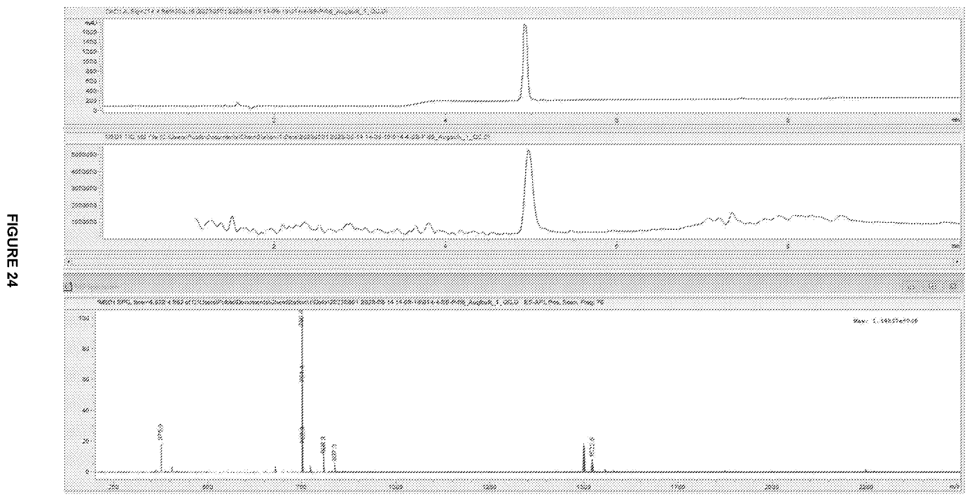

Leaf dip assays for aphids were performed with SB-P-66, and compared to a negative control vehicle and a current commercial insecticide Spirotetramat. The activity of SB-P-66 is shown in FIGS. 25 and 26; SB-P-66 has good activity whilst being eco-friendly and targeted to specific pests unlike broad spectrum chemical insecticides such as Spirotetramat.

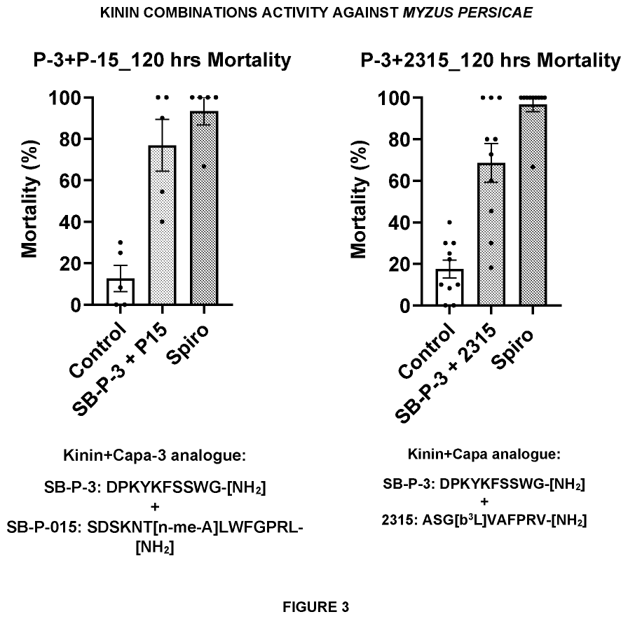

Leaf dip assays for Plutella xylostella were performed with SB-P-70, and compared to a negative control vehicle and a current commercial insecticide Chlorantraniliprole. The activity of SB-P-70 is shown in FIG. 6; SB-P-70 has good activity whilst being eco-friendly and targeted to specific pests unlike broad spectrum chemical insecticides such as Chlorantraniliprole

Lepidoptera Mortality Assay Against Spodoptera frugiperda

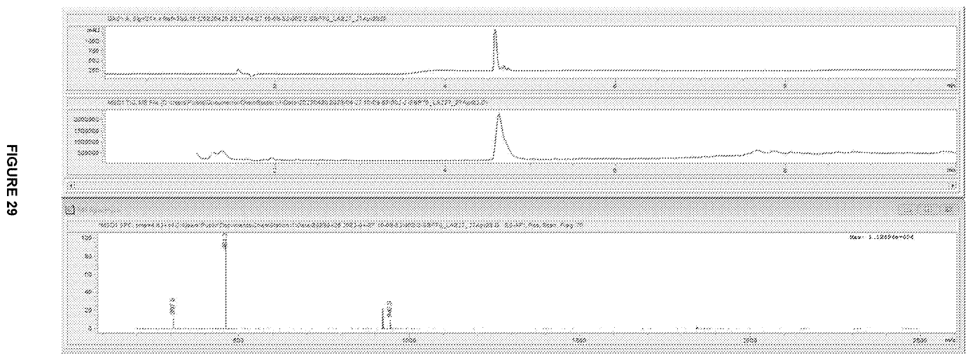

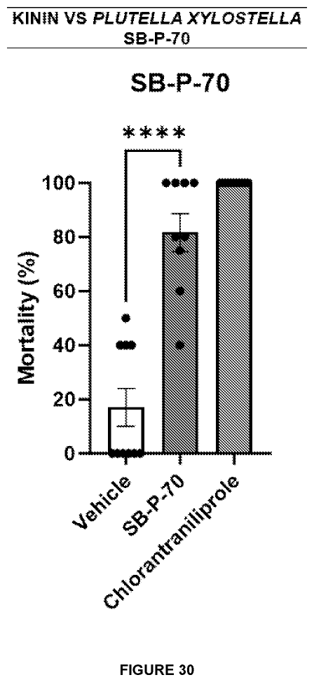

Frontier Lepidoptera Artificial diet was made up by preparing agar at 1% and then mixing it with artificial diet powder. Peptide solution was aliquoted in 10 cells and diet poured straight onto it. The final concentration of the peptide was 1×10⁻⁴ M, and each peptide was aliquoted in 10 cells. Once the diet solidified and cooled down, one L1 larvae was infested in each cell. The trays were sealed with heat and let larva were allowed to develop for 168 hrs. Finally, pupation and mortality were scored for. The lepidoptera mortality assay set up is shown in FIG. 31. Lepidoptera mortality assays were performed with SB-P-70, and compared to a negative control vehicle and the insecticide dichlorodiphenyltrichloroethane (DDT). The activity of SB-P-70 is shown in FIG. 32; SB-P-70 has good activity whilst being eco-friendly and targeted to specific pests unlike broad spectrum chemical insecticides such as DDT.

### Bee Survival Studies

Bee Oral Testing Protocol, Adapted from OECD Test No. 247: Bumblebee, Acute Oral Toxicity Test

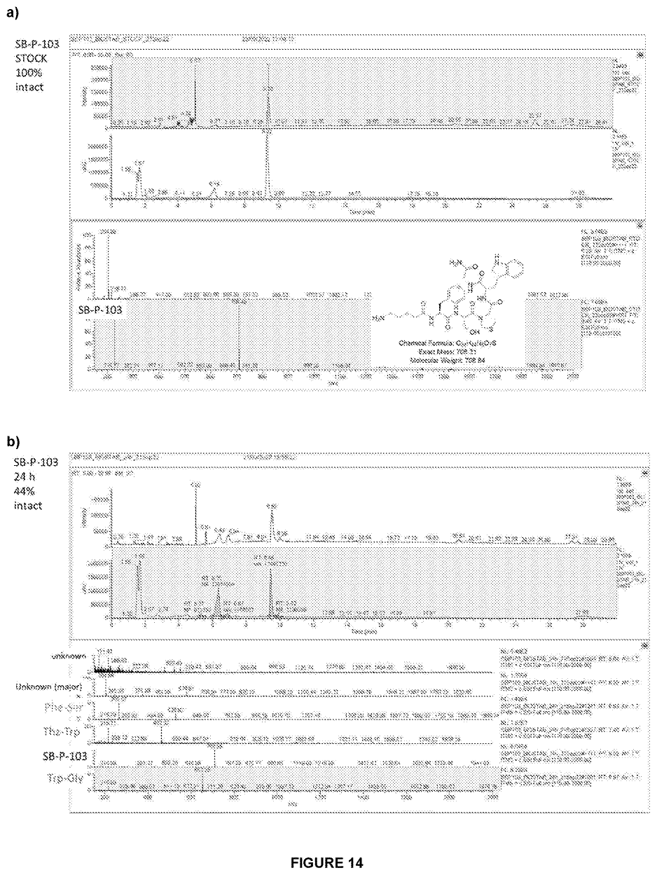

Survival of bees against peptides SB-P-065 and SB-P-066 was measured following the protocol in OECD Test 247. Results are shown in FIG. 16.

Bee oral testing protocol was carried out adapted from OECD Test No. 247: Bumblebee, Acute Oral Toxicity Test. The toxicity of SB-P-65 and SB-P-66 was compared with a negative control of sucrose or vehicle, and with a current commercial insecticide Imidacloprid. The toxicity data is shown in FIG. 16; SB-P-65 and SB-P-66 have far lower toxicity to key pollinator species such as bees compared to broad spectrum chemical insecticides such as Imidacloprid.

### Sequences

| Reference | | |
|---|---|---|
| Number | Sequence | SEQ ID NO |
| SB-P-003 | Hy-DPKYKFSSWG-[NH₂] | SEQ ID NO: 59 |
| SB-P-004 | Hy-AKFSSWG-[NH₂] | SEQ ID NO: 60 |
| SB-P-018 | Hy-ARFSSWA-[NH₂] | SEQ ID NO: 61 |
| SB-P-019 | Hy-SKFNSWA-[NH₂] | SEQ ID NO: 62 |
| SB-P-020 | Hy-AKFSSWA-[NH₂] | SEQ ID NO: 63 |
| SB-P-021 | Hy-ARFNSWA-[NH₂] | SEQ ID NO: 64 |
| SB-P-022 | Hy-VPFNSWA-[NH₂] | SEQ ID NO: 65 |
| SB-P-023 | Hy-PIFSSWG-[NH₂] | SEQ ID NO: 66 |
| SB-P-024 | Hy-GPDFYAWG-[NH₂] | SEQ ID NO: 67 |
| SB-P-032 | Ac-KQrFH[Aib]WG-[NH₂] | SEQ ID NO: 18 |
| SB-P-033 | Ac-NSVVLGKKQrFH[Aib]WG-[NH₂] | SEQ ID NO: 19 |
| SB-P-034 | Ac-FH[Aib]WG-[NH₂] | SEQ ID NO: 20 |
| SB-P-035 | Ac-KQrFHsWG-[NH₂] | SEQ ID NO: 21 |
| SB-P-036 | Ac-FHsWG-[NH₂] | SEQ ID NO: 22 |
| SB-P-052 | Hy-NSVVLGKKQ[n-me-R]FH[n-me-S]WG-[NH₂] | SEQ ID NO: 23 |
| SB-P-054 | Ac-SkFN-Aib-WA-[NH₂] | SEQ ID NO: 68 |
| SB-P-056 | Ac-SK-[n-me-F]-N-(α-Me)Ser-WA-[NH₂] | SEQ ID NO: 69 |
| SB-P-060 | Ac-PA-[n-me-F]-SsWG-[NH₂] | SEQ ID NO: 70 |
| SB-P-065 | Hy-RQKTVFSSWG-[NH₂] | SEQ ID NO: 71 |
| SB-P-066 | Hy-PAFSSWG-[NH₂] | SEQ ID NO: 72 |
| SB-P-067 | Hy-ASDKHGRPKQTFSSWG-[NH₂] | SEQ ID NO: 73 |
| SB-P-068 | Hy-VRFSPWG-[NH₂] | SEQ ID NO: 107 |
| SB-P-069 | Hy-NFSPWG-[NH₂] | SEQ ID NO: 108 |
| SB-P-070 | Hy-KVKFSAWG-[NH₂] | SEQ ID NO: 109 |
| SB-P-071 | Hy-SFSPWG-[NH₂] | SEQ ID NO: 110 |
| SB-P-072 | Hy-FSPWG-[NH₂] | SEQ ID NO: 111 |
| SB-P-073 | Hy-YFSPWG-[NH₂] | SEQ ID NO: 112 |
| SB-P-074 | Hy-KVKFSVWG-[NH₂] | SEQ ID NO: 113 |
| SB-P-075 | Hy-KVKFSTWG-[NH₂] | SEQ ID NO: 114 |
| SB-P-076 | Hy-VRFSPWG-[NH₂] | SEQ ID NO: 115 |
| SB-P-100 | Ac-PA-[n-me-F]-S-βA-WG-[NH₂] | SEQ ID NO: 74 |
| SB-P-101 | Ac-PA-[n-me-F]-S-(α-Me)S-WG-[NH₂] | SEQ ID NO: 75 |
| SB-P-102 | Ava-FS-βA-WG-[NH₂] | SEQ ID NO: 116 |
| SB-P-103 | Ava-FS-Thz-WG-[NH₂] | SEQ ID NO: 117 |
| SB-P-143 | Hy-DPAFNSWG-[NH₂] | SEQ ID NO: 76 |
| SB-P-144 | Hy-SSAFNSWG-[NH₂] | SEQ ID NO: 77 |
| SB-P-145 | Hy-RAFTSTGSSRSPAFSSWG-[NH₂] | SEQ ID NO: 78 |
| SB-P-146 | Hy-PASFSSWG-[NH₂] | SEQ ID NO: 79 |
| SB-P-147 | Hy-DAGFSSWG-[NH₂] | SEQ ID NO: 80 |
| SB-P-148 | Hy-SSGFSSWG-[NH₂] | SEQ ID NO: 81 |
| SB-P-149 | Hy-DPVFKSWG-[NH₂] | SEQ ID NO: 82 |
| SB-P-150 | Hy-DAAFSSWG-[NH₂] | SEQ ID NO: 83 |
| SB-P-151 | Hy-GSGFSSWG-[NH₂] | SEQ ID NO: 84 |
| SB-P-154 | Hy-DDDSDVSESGSKFSSWG-[NH₂] | SEQ ID NO: 85 |
| SB-P-155 | Hy-SFSSWG-[NH₂] | SEQ ID NO: 86 |
| SB-P-156 | Hy-GFSSWG-[NH₂] | SEQ ID NO: 87 |
| SB-P-157 | Hy-LDRSEGRVSKALFSSWG-[NH₂] | SEQ ID NO: 88 |
| SB-P-158 | Hy-WGQSSVGAVFSSWG-[NH₂] | SEQ ID NO: 89 |
| SB-P-159 | Hy-GAEFYSWG-[NH₂] | SEQ ID NO: 90 |
| SB-P-169 | Hy-FSSWG-[NH₂] | SEQ ID NO: 91 |
| N/A | Hy-GPAFSSWG-[NH₂] | SEQ ID NO: 92 |
| N/A | Hy-AAAFNSWG-[NH₂] | SEQ ID NO: 93 |
| N/A | Hy-NAAFSSWG-[nh2] | SEQ ID NO: 94 |
| N/A | Hy-NAAFNSWG-[NH₂] | SEQ ID NO: 95 |
| N/A | Hy-VPAFNSWG-[NH₂] | SEQ ID NO: 96 |
| N/A | Hy-TNTAFSSWG-[NH₂] | SEQ ID NO: 97 |
| N/A | Hy-PRFYSWG-[NH₂] | SEQ ID NO: 98 |
| N/A | Hy-GADFYAWG-[NH₂] | SEQ ID NO: 99 |
| N/A | [Ac]-SK[n-me-F]N[βAla]WA-[NH₂] | SEQ ID NO: 100 |

### REFERENCES

A number of publications are cited in order to more fully describe and disclose the invention and the state of the art to which the invention pertains. Full citations for these references are provided below. The entirety of each of these references is incorporated herein by reference.
Koyama T, Terhzaz S, Naseem M T, Nagy S, Rewitz K, Dow J A T, Davies S A, Halberg K V. A nutrient-responsive hormonal circuit mediates an inter-tissue program regulating metabolic homeostasis in adult Drosophila. Nat Commun. 2021
Kean L, Cazenave W, Costes L, Broderick K E, Graham S, Pollock V P, Davies S A, Veenstra J A, Dow J A. Two nitridergic peptides are encoded by the gene capability in Drosophila melanogaster. Am J Physiol Regul Integr Comp Physiol. 2002
Yeoh J G C, Pandit A A, Zandawala M, Nassel D R, Davies S A, Dow J A T. DINeR: Database for Insect Neuropeptide Research. Insect Biochem Mol Biol. 2017
Ahn S J, Corcoran J A, Vander Meer R K, Choi M Y. Identification and Characterization of GPCRs for Pyrokinin and CAPA Peptides in the Brown Marmorated Stink Bug, Halyomorpha halys (Hemiptera: Pentatomidae). Front Physiol. 2020
Audsley N and Down R E, G protein coupled receptors as targets for next generation pesticides. Insect Biochem Molec 67: 27-37 (2015).
Halberg K A, Terhzaz S, Cabrero P, Davies S A and Dow J A T, Tracing the evolutionary origins of insect renal function. Nat Commun 6 (2015).
Dow J A, Insights into the Malpighian tubule from functional genomics. J Exp Biol 212: 435-445 (2009).
Huesmann G R, Cheung C C, Loi P K, Lee T D, Swiderek K M and Tublitz N J, Amino acid sequence of CAP2b, an insect cardioacceleratory peptide from the tobacco hawkmoth Manduca sexta. FEBS Lett 371: 311-314 (1995).
Davies S A, Cabrero P, Povsic M, Johnston N R, Terhzaz S and Dow J A T, Signaling by Drosophila capa neuropeptides. Gen Comp Endocr 188: 60-66 (2013).
Terhzaz S, Teets N M, Cabrero P, Henderson L, Ritchie M G, Nachman R J, Dow J A T, Denlinger D L and Davies S A, Insect capa neuropeptides impact desiccation and cold tolerance. Proc Natl Acad Sci 201501518 (2015).
Terhzaz S, Alford L, Yeoh J G C, Marley R, Dornan A T, Dow J A T and Davies S A, Renal neuroendocrine control of desiccation and cold tolerance by Drosophila suzukii. Pest Manag Sci 74: 800-810 (2017).
Predel R, Wegener C, Biology of the CAPA peptides in insects. Cell Mol Life Sci 63: 2477-2490 (2006).
Lamango N S, Nachman R J, Hayes T K, Strey A and Isaac R E, Hydrolysis of insect neuropeptides by an angiotensin converting enzyme from the housefly, M. domestica. Peptides 18: 47-52 (1997).
Terhzaz S, Cabrero P, Robben J H, Radford J C, Hudson B D, Milligan G, Dow J A and Davies S A, Mechanism and function of Drosophila capa GPCR: a desiccation stress-responsive receptor with functional homology to human neuromedinU receptor. PloS One 7(1): e29897 (2012).
Blackman R L and Eastop V F, Aphids on the World's Crops: An Identification Guide, John Wiley & Sons Ltd, Chichester, UK. (2000).
Dow J A T, Maddrell S H P, Gortz A, Skaer N J V, Brogan S and Kaiser K, The Malpighian tubules of Drosophila melanogaster—a novel phenotype for studies of fluid secretion and its control. J Exp Biol 197: 421-428 (1994).
Davies S A, Huesmann G R, Maddrell S H, O'Donnell M J, Skaer N J, Dow J A T and Tublitz N J, CAP2b, a cardioacceleratory peptide, is present in Drosophila and stimulates tubule fluid secretion via Cgmp. Am J Physiol 269: R1321-R1326 (1995).
Beyenbach K W, Skaer H and Dow J A, The developmental, molecular, and transport biology of Malpighian tubules. Annu Rev Entomol 55: 351-374 (2010).
Sadeghi A., Van Damme, E. J. M. and Smagghe G. (2009) Evaluation of the susceptibility of the pea aphid, Acyrthosiphon pisum, to a selection of novel biorational insecticides using an artificial diet. Journal of Insect Science 9:65.
Van Emden F (2009). Artificial diet for aphids—thirty years' experience. REDIA, XCII: 163-167

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

### Numbered Paragraphs

The following numbered paragraphs define particular aspects and embodiments of the invention.

1. An insecticidal compound having the formula (I) below, or a salt or solvate thereof:

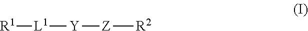

wherein:
R¹ is hydrogen (which may be designated “H-” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}, —C(═N⁺(R^{1b})(R^{1c}))NR^{1d}R^{1e} acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl, biotin, a sugar moiety or (poly)alkyleneglycol;
wherein each of R^{1a}, R^{1b}, R^{1c}, R^{1d} and R^{1e} is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moiety;
L¹ is absent or is:
   *—(C═O)C₁₋₆-alkylene-NH— where * denotes the point of attachment to Y or Z;
   (C₁₋₂₀)alkylene,
   (C₂₋₂₀)alkenylene,
   (poly)alkyleneglycol;
   wherein L¹ is optionally substituted with one or more groups selected from oxo (═O), halogen, ═N and ═S;
Y is absent or is a peptide comprising from 1 to 16 amino acids; wherein Y optionally comprises a (poly)alkyleneglycol linker in the peptide sequence;
Z is a peptide of the formula:

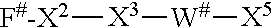

wherein:
   X² is selected from S^{#}, Y^{#}, N^{#} or H^{#};
   X³ is selected from S^{#}, A^{#}, P^{#}, V^{#} or T^{#},
   X⁵ is selected from G^{#}, A^{#};
   wherein “^{#}” indicates that the listed residue is independently the unmodified amino acid, a modified amino acid or a non-natural amino acid analogue; and
R² is NH₂, NR^{2a}H, NR^{2a}R^{2b}, or OR^{2a}; wherein each of R^{2a} and R^{2b} if present are independently C₁₋₆-alkyl (e.g. methyl, ethyl, propyl, butyl, pentyl or hexyl), C₃₋₆-alkenyl, C₆₋₁₆_aryl, C₆₋₁₆_aryl-C₁₋₆-alkyl, C₁₋₆-alkylene-C₆₋₁₆-aryl, or C₁₋₆-haloalkyl, each of which may optionally be substituted with one or more groups selected from halogen, C₁₋₆-alkyl, or C₁₋₆-haloalkyl.

2. An insecticidal compound according to paragraph 1, wherein Z is a peptide selected from Z^{a}, Z^{b} or Z^{c};
wherein:
a) Z^{a} is a peptide of the formula:

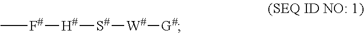

b) Z^{b} is a peptide of the formula:

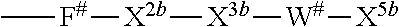

wherein:
X^{2b} is selected from amino acid residues S^{#}, Y^{#}, N^{#} or H^{#};
X^{3b} is selected from amino acid residues S^{#}, A^{#}, P^{#}, V^{#} or T^{#} (optionally X^{3b} is selected from amino acid residues S^{#} or A^{#});
X^{5b} is selected from amino acid residues A^{#} or G^{#}, or
c) Z^{c} is a peptide of the formula:

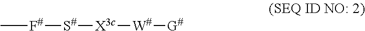

wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue.

3. An insecticidal compound according to paragraph 1 or paragraph 2, wherein Y is absent or is a peptide comprising from 1 to 14 amino acids.

4. The insecticidal compound according to any one of the preceding paragraphs, wherein R¹ is selected from:
hydrogen;
acyl optionally substituted with a sugar moiety;
fatty acyl;
heteroaryl;
—NHC₆₋₁₆Aryl;
a sugar moiety;
biotin; or
(poly)ethyleneglycol of the formula *—(OCH₂CH₂)ₙ—Rₚ, where * denotes the point of attachment to L¹, Y or Z, n is an integer from 1 to 16, and Rₚ is selected from —NH₂, —OH or —Ome.

5. The insecticidal compound according to any one of the preceding paragraphs, wherein R¹ is selected from:
hydrogen;
acyl optionally substituted with a sugar moiety, wherein the acyl group is selected from formyl, acetyl (Ac), propanoyl, butanoyl;
fatty acyl selected from palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl;
indolyl;
—NHC₆₋₁₆Aryl;
a monosaccharide or disaccharide moiety;
biotin;
(poly)ethyleneglycol of the formula *—(OCH₂CH₂)ₙ—Rₚ, where * denotes the point of attachment to L¹, Y or Z, n is an integer from 4 to 12, and Rₚ is selected from —NH₂, —OH or —Ome.

6. The insecticidal compound according to any one of the preceding paragraphs, wherein R¹ is hydrogen or acetyl.

7. The insecticidal compound according to any one of the preceding paragraphs, wherein L¹ is absent or is:
—(C═O)C₄₋₁₂-alkylene-NH—, where * denotes the point of attachment to Y or Z;
—(C═O)—C₁₋₄alkylene-(C═O)—.

8. The insecticidal compound according to any one of the preceding paragraphs, wherein L¹ is absent or is:

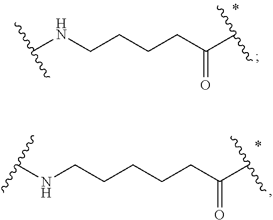 [SMILES: *NCCCCC(*)=O ; *NCCCCCC(*)=O]

where * denotes the point of attachment to Y or Z; or

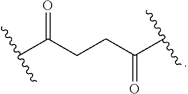 [SMILES: *C(=O)CCC(*)=O]

9. The insecticidal compound according to any one of the preceding paragraphs, wherein L¹ is absent.

10. The insecticidal compound according to any one of the preceding paragraphs, wherein the Y—Z moiety comprises at least one modified amino acid or a non-natural amino acid analogue.

11. The insecticidal compound according to any one of the preceding paragraphs, wherein the modified amino acid or non-natural amino acid analogue in peptides Y and Z is selected from:
i. an N-methylated amino acid;
ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
iii. an α-alkylated (e.g. α-methylated) amino acid
iv. a D-amino acid;
v. a beta-amino acid;
vi. a peptoid amino acid analogue;
vii. a sugar modified amino acid; or
viii. a biotin modified amino acid.

12. The insecticidal compound according to any one of the preceding paragraphs, wherein the modified amino acid or non-natural amino acid analogue in peptides Y and Z is selected from:
i. an N-methylated amino acid;
ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
iii. an α-methylated amino acid;
iv. a D-amino acid; or
v. a beta amino acid.

13. The insecticidal compound according to any one of the preceding paragraphs, wherein the modified amino acid or non-natural amino acid analogue in peptides Y and Z is selected from:
i. an N-methylated amino acid;
ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
iii. an α-methylated amino acid;
iv. a D-amino acid.

14. The insecticidal compound according to any one of the preceding paragraphs, wherein the modified or non-natural amino acid is selected from:
i. an N-methylated amino acid;
ii. α-aminoisobutyric acid (Aib);
iii. thiazolidine-4-carboxylic acid (Thz);
iv. an α-methylated amino acid
v. a D-amino acid.

15. The insecticidal compound according to any one of the preceding paragraphs, wherein R² is NH₂, NR^{2a}H or NR^{2a}R^{2b}, wherein each of R^{2a} and R^{2b} if present is independently C₁₋₆-alkyl (e.g. methyl, ethyl, propyl, butyl, pentyl or hexyl).

16. The insecticidal compound according to any one of the preceding paragraphs, wherein R² is NH₂.

17. An insecticidal compound having the formula (Ia) below:

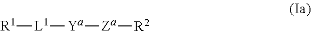

wherein:
   R¹, L¹, and R² are as defined in any one of the preceding paragraphs;
   Y^{a} is a group Y as defined in any one of the preceding paragraphs, optionally wherein Y^{a} is absent or is a peptide comprising from 1 to 12 amino acids;
   Z^{a} is a peptide of the formula:

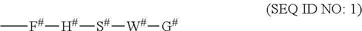

wherein “^{#}” indicates that the residues “F”, “H”, “S”, “W” and “G” are independently the unmodified amino acid, a modified amino acid or a non-natural amino acid analogue.

18. The insecticidal compound according to paragraph 19, wherein at least one of the residues in the motif Y^{a}—Z^{a} is a modified amino acid or non-natural amino acid analogue; optionally with the proviso that if the S^{#} residue is an α-aminoisobutyric acid group ([Aib]), then the motif Y^{a}—Z^{a} further comprises at least one D-amino acid.

19. The insecticidal compound according to paragraph 19 or 20, wherein the “S^{#}” residue in the Z motif is a modified amino acid or non-natural amino acid analogue.

20. The insecticidal compound according to any one of paragraphs 19 to 21, wherein Y is absent or is a peptide of the formula:

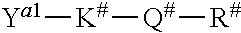

wherein Y^{a1} is absent or a peptide comprising from 1 to 9 amino acids;
wherein “^{#}” indicates that the residue is either a naturally occurring amino acid, a modified amino acid or a non-natural amino acid analogue as defined in any one of paragraphs 13 to 16.

21. The insecticidal compound according to any one of paragraphs 17 to 20, wherein Y—Z is a peptide of the formula:

| (SEQ ID NO: 13) | |
|---|---|
| | Y^{a1}-K^{#}-Q^{#}-R^{#}-F-H^{#}-S^{#}-W-G; |
| | or |
| (SEQ ID NO: 138) | |
| | F-H^{#}-S^{#}-W-G |

wherein Y^{a1} is absent or a peptide comprising from 1 to 9 amino acids;
“^{#}” indicates that the residues “K”, “Q”, “R”, “H” and “S” are independently an unmodified amino acid, a modified amino acid or non-natural amino acid analogue;
wherein at least one of the residues “K”, “Q”, “R”, “H” and “S” is a modified amino acid or non-natural amino acid analogue as defined in any one of paragraphs 13 to 16.

22. The insecticidal compound according to any one of paragraphs 17 to 21, wherein Y—Z is a peptide of the formula:

| (SEQ ID NO: 139) | |
|---|---|
| | Y^{a1}-K-Q-R^{#-}F-H^{#}-S^{#}-W-G; |
| | or |
| (SEQ ID NO: 138) | |
| | F-H^{#}-S^{#}-W-G |

wherein Y^{a1} is absent or a peptide comprising from 1 to 9 amino acids;
wherein “^{#}” indicates that the residues “R”, “H” and “S” are independently an unmodified amino acid, a modified amino acid or non-natural amino acid analogue;
wherein at least one of the residues “R”, “H” and “S” is a modified amino acid or non-natural amino acid analogue as defined in any one of paragraphs 13 to 16;
   optionally wherein Y^{a}—Z is a peptide of the formula:

| (SEQ ID NO: 15) | |
|---|---|
| | Y^{a1}-K-Q-R^{#}-F-H-S^{#}-W-G; |
| | or |
| (SEQ ID NO: 7) | |
| | F-H-S^{#}-W-G. |

Wherein Y^{a1} is absent or a peptide comprising from 1 to 9 amino acids;
wherein “^{#}” indicates that the residues “R”, and “S” are independently an unmodified amino acid, a modified amino acid or non-natural amino acid analogue;
wherein at least one of the residues “R”, if present, and “S” is a modified amino acid or non-natural amino acid analogue as defined in any one of paragraphs 13 to 16.

23. The insecticidal compound according to any one of paragraphs 17 to 22, wherein the “S^{#}” residue in the Y^{a} motif is N-methyl serine ([n-me-S]] or D-serine ([s]).

24. The insecticidal compound according to any one of the preceding paragraphs, wherein the Z moiety has the formula:

| (SEQ ID NO: 3) | |
|---|---|
| | FH[Aib]WG |
| (SEQ ID NO: 4) | |
| | FHSWG; |
| | or |
| (SEQ ID NO: 5) | |
| | FH[n-me-S]WG. |

25. A compound having the formula:

| (SEQ ID NO: 18) | |
|---|---|
| | Ac-KQrFH[Aib]WG-[NH₂]; |
| (SEQ ID NO: 19) | |
| | Ac-NSVVLGKKQrFH[Aib]WG-[NH₂]; |
| (SEQ ID NO: 20) | |
| | Ac-FH[Aib]WG-[NH₂]; |
| (SEQ ID NO: 21) | |
| | Ac-KQrFHsWG-[NH₂]; |
| (SEQ ID NO: 22) | |
| | Ac-FHsWG-[NH₂]; |
| (SEQ ID NO: 23) | |
| | NSVVLGKKQ[n-me-R]FH[n-me-S]WG-[NH₂]; |

or a salt thereof.

26. An insecticidal compound having the formula (Ib) below:

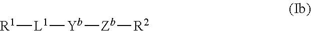

wherein:
R¹, L¹, and R² are as defined in any one of the preceding paragraphs;
Y^{b} is a group Y as defined herein, optionally wherein Y^{b} is absent or is a peptide comprising from 1 to 14 amino acids;
Z^{b} is a peptide of the formula:

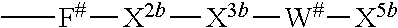

wherein:
X^{2b} is selected from amino acid residues S^{#}, Y^{#} N^{#} or H #
X^{3b} is selected from amino acid residues S^{#}, A^{#}, P^{#}, V^{#} or T^{#};
X^{5b} is selected from amino acid residues A^{#} or G^{#},
wherein “^{#}” indicates that the listed residue is independently the unmodified amino acid, a modified amino acid or a non-natural amino acid analogue as defined in any one of the preceding paragraphs.

27. An insecticidal compound according to paragraph 28, wherein Z^{b} is a peptide with a formula selected from any one of (ZbI) to (ZbVI) (sub formulae of Z^{b}):

| | (ZbI) |
|---|---|
| (SEQ ID NO: 24) | |
| | F^{#}-S^{#}-S^{#}-W^{#}-G^{#;} |
| | (ZbII) |
| (SEQ ID NO: 25) | |
| | F^{#}-S^{#}-S^{#}-W^{#}-A^{#;} |
| | (ZbIII) |
| (SEQ ID NO: 26) | |
| | F^{#}-N^{#}-S^{#}-W^{#}-A^{#;} |
| | (ZbIV) |
| (SEQ ID NO: 27) | |
| | F^{#}-Y^{#}-S^{#}-W^{#}-G^{#;} |
| | (ZbV) |
| (SEQ ID NO: 28) | |
| | F^{#}-N^{#}-A^{#}-W^{#}-A^{#;} |
| | or |
| | (ZbVI) |
| (SEQ ID NO: 29) | |
| | F^{#}-S^{#}-A^{#}-W^{#}-G^{#;} |

wherein any residue marked with a “^{#}” is independently either the listed unmodified amino acid, a modified amino acid or a non-natural amino acid analogue as defined in any one of the preceding paragraphs;
optionally Z^{b} is a peptide with a formula selected from any one of (ZbI-I) to (ZbVI-I) (sub formulae of Z^{b}):

| | (ZbI-I) |
|---|---|
| (SEQ ID NO: 32) | |
| | F^{#}-S-S^{#}-W-G; |
| | (ZbII-I) |
| (SEQ ID NO: 33) | |
| | F^{#}-S-S^{#}-W-A; |
| | (ZbIII-I) |
| (SEQ ID NO: 34) | |
| | F^{#}-N-S^{#}-W-A; |
| | (ZbIV-I) |
| (SEQ ID NO: 35) | |
| | F^{#}-Y-S^{#}-W-G; |
| | (ZbV-I) |
| (SEQ ID NO: 36) | |
| | F^{#}-N-A^{#}-W-A; |
| | or |
| | (ZbVI-I) |
| (SEQ ID NO: 37) | |
| | F^{#}-S-A^{#}-W-G; |

wherein any residue marked with a “^{#}” is independently either the listed unmodified amino acid, a modified amino acid or a non-natural amino acid analogue as defined in any one of the preceding paragraphs.

28. An insecticidal compound according to paragraph 26 or 27, wherein the modified or non-natural amino acid analogues are selected from an N-methylated amino acid, aminoisobutyric acid (Aib), a D-amino acid or a beta-amino acid.

29. An insecticidal compound according to any one of paragraphs 28 to 30, wherein Z^{b} is selected from:

| (SEQ ID NO: 40) | |
|---|---|
| | FSSWG |
| (SEQ ID NO: 41) | |
| | FSSWA |
| (SEQ ID NO: 42) | |
| | FNSWA |
| (SEQ ID NO: 43) | |
| | FYAWG |
| (SEQ ID NO: 44) | |
| | FN[Aib]WA |
| (SEQ ID NO: 45) | |
| | FN-BA-WA |
| (SEQ ID NO: 46) | |
| | (n-me-F)-SsWG |
| (SEQ ID NO: 47) | |
| | (N-me-F)-S-βA-WG |
| (SEQ ID NO: 48) | |
| | (N-me-F)-S-(α-Me)S-WG; |

Y^{b} is selected from:

| (SEQ ID NO: 51) | |
|---|---|
| | DPKYK; |

AK;
AR;
SK;
VP;
PI;
GPD;
Sk;

| (SEQ ID NO: 52) | |
|---|---|
| | RQKTV; |

PA; or

| (SEQ ID NO: 53) | |
|---|---|
| | ASDKHGRPKQT. |

30. An insecticidal compound having the formula (Ic) below:

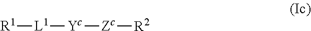

wherein:
   R¹, L¹, and R² are as defined in any one of the preceding paragraphs;
   Y^{c} is a group Y as defined in any one of the preceding paragraphs, optionally wherein
   Y^{c} is absent or is a peptide comprising from 1 to 12 amino acids; and
   Z^{c} is a peptide of the formula:

| (SEQ ID NO: 2) | |
|---|---|
| | -F^{#}-S^{#}-X^{3c}-W^{#}-G^{#} |

wherein X^{3c} is selected from amino acid residues A^{#}, P^{#}, V^{#} or T^{#};
wherein “^{#}” indicates that the listed residue is independently the unmodified amino acid, a modified amino acid or a non-natural amino acid analogue.

31. An insecticidal compound according to paragraph 31, wherein Z^{c} is a peptide of the formula:

| | (ZcI) |
|---|---|
| (SEQ ID NO: 118) | |
| | -F-S-X^{3c}-W-G |

and X^{3c} is selected from amino acid residues A^{#}, P^{#}, V^{#} or T^{#}; optionally wherein X^{3c} is P^{#} or V^{#}.

32. An insecticidal compound according to paragraph 30 or 31, wherein Z^{c} is a peptide of the formula selected from ZcI-I to ZcI-IV:

| | (ZcI-I) |
|---|---|
| (SEQ ID NO: 119) | |
| | -F-S-A^{#}-W-G |
| | (ZcI-II) |
| (SEQ ID NO: 120) | |
| | -F-S-P^{#}-W-G |
| | (ZcI-III) |
| (SEQ ID NO: 121) | |
| | -F-S-V^{#}-W-G |
| | (ZcI-IV) |
| (SEQ ID NO: 122) | |
| | -F-S-T^{#}-W-G |

33. An insecticidal compound according to any one of paragraphs 31 to 33, wherein the one or more modified amino acids or a non-natural amino acid analogues is independently selected from an N-methylated amino acid, aminoisobutyric acid (Aib), thiazolidine-4-carboxylic acid (Thz), a beta amino acid or a D-amino acid.

34. An insecticidal compound according to any one of paragraphs 30 to 33, wherein Z^{c} is a peptide of the formula selected from:

| (SEQ ID NO: 101) | |
|---|---|
| | FSPWG |
| (SEQ ID NO: 102) | |
| | FSAWG |
| (SEQ ID NO: 103) | |
| | FSVWG |
| (SEQ ID NO: 104) | |
| | FSTWG |
| (SEQ ID NO: 105) | |
| | FS-BA-WG |
| (SEQ ID NO: 106) | |
| | FS-Thz-WG; |

and/or
Y^{c} is absent or selected from:
   VR;
   N;
   KVK;
   SFS; or
   Y.

35. An insecticidal compound selected from:

(SEQ ID NO: 59)
DPKYKFSSWG-[NH₂];
(SEQ ID NO: 60)
AKFSSWG-[NH₂];
(SEQ ID NO: 61)
ARFSSWA-[NH₂];
(SEQ ID NO: 62)
SKFNSWA-[NH₂];
(SEQ ID NO: 63)
AKFSSWA-[NH₂];
(SEQ ID NO: 64)
ARFNSWA-[NH₂];
(SEQ ID NO: 65)
VPFNSWA-[NH₂];
(SEQ ID NO: 66)
PIFSSWG-[NH₂];
(SEQ ID NO: 67)
GPDFYAWG-[NH₂];
(SEQ ID NO: 18)
Ac-KQrFH[Aib]WG-[NH₂];
(SEQ ID NO: 19)
Ac-NSVVLGKKQrFH[Aib]WG-[NH₂];
(SEQ ID NO: 20)
Ac-FH[Aib]WG-[NH₂];
(SEQ ID NO: 21)
Ac-KQrFHsWG-[NH₂];
(SEQ ID NO: 22)
Ac-FHsWG-[NH₂];
(SEQ ID NO: 23)
NSVVLGKKQ[n-me-R]FH[n-me-S]WG-[NH₂];
(SEQ ID NO: 68)
Ac-SKFN-Aib-WA-[NH₂];
(SEQ ID NO: 69)
Ac-SK-[n-me-F]-N-(α-Me) Ser-WA-[NH₂];
(SEQ ID NO: 70)
Ac-PA-[n-me-F]-SsWG-[NH₂];
(SEQ ID NO: 71)
RQKTVFSSWG-[NH₂];
(SEQ ID NO: 72)
PAFSSWG-[NH₂];
(SEQ ID NO: 73)
ASDKHGRPKQTFSSWG-[NH₂];
(SEQ ID NO: 107)
VRFSPWG-[NH₂];
(SEQ ID NO: 108)
NFSPWG-[NH₂];
(SEQ ID NO: 109)
KVKFSAWG-[NH₂];
(SEQ ID NO: 110)
SFSPWG-[NH₂];
(SEQ ID NO: 111)
FSPWG-[NH₂];
(SEQ ID NO: 112)
YFSPWG-[NH₂];
(SEQ ID NO: 113)
KVKFSVWG-[NH₂];
(SEQ ID NO: 114)
KVKFSTWG-[NH₂];
(SEQ ID NO: 74)
Ac-PA-[n-me-F]-S-βa-WG-[NH₂];
(SEQ ID NO: 75)
Ac-PA-[n-me-F]-S-(α-Me)S-WG-[NH₂];
(SEQ ID NO: 116)
Ava-FS-BA-WG-[NH₂];
(SEQ ID NO: 117)
Ava-FS-Thz-WG-[NH₂];

or a salt or solvate thereof.

36. A composition, e.g. an insect control composition or plant protection composition, comprising a compound according to any one of the preceding paragraphs in admixture with one or more solvents, carriers, diluents, adjuvants, preservatives, dispersants, emulsifying agents, or synergists.

37. A composition according to paragraph 36 which is an aqueous composition.

38. A composition according to paragraph 36 or 37, further comprising one or more of a kinin peptide, an AKH peptide, a DH31 peptide, a DH44 peptide, a pyrokinin peptide or a CAPA peptide (e.g. a CAPA-1, CAPA-2 or CAPA-3 analogue)

39. Use, as an insect control agent, of a compound according to any one of paragraphs 1 to 35, or a composition according to paragraph 36, 37 or 38; optionally wherein said use is against insects of the order diptera, hemiptera or lepidoptera.

40. The use according to paragraph 39 wherein said use is as an insecticide against:
a) diptera insects, wherein Z is a group Z^{a} as defined in paragraph 2;
b) hemiptera insects (e.g. Halyomorpha halys or Myzus persicae insects), wherein Z is a group Z^{b} as defined in paragraph 2;
c) lepidoptera insects (e.g. Plutella xylostella), wherein Z is a group Z^{c} as defined in paragraph 2;

41. A method of increasing insect mortality, the method comprising contacting an insect or insect population with a compound according to any one of paragraphs 1 to 35, or a composition according to paragraph 36, 37 or 38; wherein:
a) the insect population is a diptera insect population, optionally wherein Z is a group Z^{a} as defined in paragraph 2;
b) the insect population is a hemiptera insect population (e.g. Halyomorpha halys or Myzus persicae), optionally wherein Z is a group Z^{b} as defined in paragraph 2;
c) the insect population is a lepidoptera insect population (e.g. Plutella xylostella), optionally wherein Z is a group Z^{c} as defined in paragraph 2;

42. Use of a compound according to any one of paragraphs 1 to 35, or a composition according to paragraph 36, 37 or 38, as a plant protection agent, for protecting a plant against:
a) diptera insects, optionally wherein Z is a group Z^{a} as defined in paragraph 2;
b) hemiptera insects (e.g. Halyomorpha halys or Myzus persicae insects), optionally wherein Z is a group Z^{b} as defined in paragraph 2;
c) lepidoptera insects (e.g, Plutella xylostella), optionally wherein Z is a group Z° as defined in paragraph 2.

43. A method of inhibiting infestation of a plant by insects, the method comprising contacting the plant with a compound as described in any one of paragraphs to 35, or a composition according to paragraph 36, 37 or 38; wherein
a) the insects are diptera insects, optionally wherein Z is a group Z^{a} as defined in paragraph 2;
b) the insects are hemiptera insects (e.g. Halyomorpha halys or Myzus persicae insects), optionally wherein Z is a group Z^{b} as defined in paragraph 2;
c) the insects are lepidoptera insects (e.g, Plutella xylostella), optionally wherein Z is a group Z^{c} as defined in paragraph 2;
optionally wherein the compound is applied to the plant while the plant is free or substantially free of insects.

44. A method of reducing insect infestation of a plant, or of reducing insect load on a plant, the method comprising contacting the plant with a compound as described in any one of paragraphs 1 to 35, or a composition according to paragraph 36, 37 or 38; wherein
a) the insects are diptera insects, optionally wherein Z is a group Z^{a} as defined in paragraph 2;
b) the insects are hemiptera insects (e.g. Halyomorpha halys or Myzus persicae insects), optionally wherein Z is a group Z^{b} as defined in paragraph 2;
c) the insects are lepidoptera insects (e.g, Plutella xylostella), optionally wherein Z is a group Z^{c} as defined in paragraph 2.

1. An insecticidal compound having the formula (XX) below, or a salt or solvate thereof: 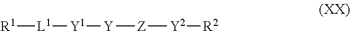
wherein:
R¹ is hydrogen (which may be designated “H-” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}, —C(═N⁺(R^{1b})(R^{1c}))NR^{1d}R^{1e}, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl, biotin, a sugar moiety or (poly)alkyleneglycol, phosphate or sulphate;
wherein each of R^{1a}, R^{1b}, R^{1c}, R^{1d} and R^{1e} is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl, a sugar moiety, phosphate or sulphate;
L¹ is absent or is:
*—(C═O)C₁₋₆-alkylene-NH— where * denotes the point of attachment to Y¹, Y or Z;
(C₁₋₂₀)alkylene,
(C₂₋₂₀)alkenylene,
(poly)alkyleneglycol;
wherein L¹ is optionally substituted with one or more groups selected from oxo (═O), halogen, ═N and ═S;
Y¹ is absent or is a peptide comprising from 1 to 2 amino acids;
Y is absent or is a peptide comprising from 1 to 16 amino acids; wherein Y optionally comprises a (poly)alkyleneglycol linker in the peptide sequence;
Y² is absent or is a peptide comprising from 1 to 2 amino acids;
Z is a peptide of the formula: 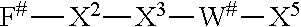
wherein:
X² is selected from S^{#}, Y^{#}, K^{#}, N^{#} or Hm;
X³ is selected from S^{#}, A^{#}, P^{#}, V^{#} or T^{#}
X⁵ is selected from G^{#}, A^{#};
wherein “^{#}” indicates that the listed residue is independently the unmodified amino acid, a modified amino acid or a non-natural amino acid analogue;
R² is NH₂, NR^{2a}H, NR^{2a}R^{2b}, OH or OR^{2a}; wherein each of R^{2a} and R^{2b} if present are independently C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkylene-C₆₋₁₆-aryl, or C₁₋₆ haloalkyl, each of which may optionally be substituted with one or more groups selected from halogen, C₁₋₆-alkyl, or C₁₋₆-haloalkyl.

2. An insecticidal compound having the formula (I) below, or a salt or solvate thereof: 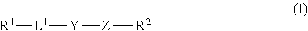
wherein:
R¹ is hydrogen (which may be designated “H-” or “Hy-”), C₁₋₄alkyl (e.g. methyl, ethyl, propyl, butyl), formyl, —N(R^{1a})—C(═N⁺(R^{1b})(R^{1c}))NR^{1d}R^{1e}, —C(═N⁺(R^{1b})(R^{1c}))NR^{1d}R^{1e}, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl, biotin, a sugar moiety or (poly)alkyleneglycol;
wherein each of R^{1a}, R^{1b}, R^{1c}, R^{1d} and R^{1e} is independently selected from hydrogen or C₁₋₄ alkyl (e.g. methyl, ethyl, propyl, butyl);
and any alkyl, formyl, acyl, fatty acyl, benzyl, benzoyl, heteroaryl or trifluoroacetyl, —NHC₁₋₁₈alkyl, —NHC₆₋₁₆Aryl, —NH—C₁₋₆alkyl-C₆₋₁₀aryl or (poly)alkyleneglycol may optionally be substituted with one or more groups selected from halogen, C₁₋₆alkyl, C₁₋₆haloalkyl or a sugar moiety;
L¹ is absent or is:
*—(C═O)C₁₋₁₆-alkylene-NH— where * denotes the point of attachment to Y or Z;
(C₁₋₂₀)alkylene,
(C₂₋₂₀)alkenylene,
(poly)alkyleneglycol;
wherein L¹ is optionally substituted with one or more groups selected from oxo (═O), halogen, ═N and ═S;
Y is absent or is a peptide comprising from 1 to 16 amino acids; wherein Y optionally comprises a (poly)alkyleneglycol linker in the peptide sequence;
Z is a peptide of the formula: 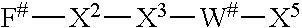
wherein:
X² is selected from S^{#}, Y^{#}, K^{#}, N^{#} or H^{#} (preferably X² is selected from S^{#}, Yn, N^{#} or H^{#})
X³ is selected from S^{#}, A^{#}, P^{#}, V^{#} or T^{#},
X⁵ is selected from G^{#}, A^{#};
wherein “^{#}” indicates that the listed residue is independently the unmodified amino acid, a modified amino acid or a non-natural amino acid analogue; and
R² is NH₂, NR^{2a}H, NR^{2a}R^{2b}, OH or OR^{2a} (preferably R² is NH₂, NR^{2a}H, NR^{2a}R^{2b}, or OR^{2a});
wherein each of R^{2a} and R^{2b} if present are independently C₁₋₆-alkyl (e.g. methyl, ethyl, propyl, butyl, pentyl or hexyl), C₃₋₆-alkenyl, C₆₋₁₆-aryl, C₆₋₁₆-aryl-C₁₋₆-alkyl, C₁₋₆-alkylene-C₆₋₁₆-aryl, or C₁₋₆-haloalkyl, each of which may optionally be substituted with one or more groups selected from halogen, C₁₋₆-alkyl, or C₁₋₆-haloalkyl.

3. An insecticidal compound according to claim 1, wherein Y¹ and Y² are absent.

4. An insecticidal compound according to claim 1, wherein Z is a peptide selected from Z^{a}, Z^{b} or Z^{c};
wherein:
a) Z^{a} is a peptide of the formula:
(SEQ ID NO: 1)
-F^{#}-H^{#}-S^{#}-W^{#}-G^{#};
b) Z^{b} is a peptide of the formula: 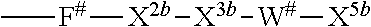
wherein:
X^{2b} is selected from amino acid residues S^{#}, Y^{#}, K^{#}, N^{#} or H^{#};
X^{3b} is selected from amino acid residues S^{#}, A^{#}, P^{#}, V^{#} or T^{#} (optionally S^{#} or A^{#});
X^{5b} is selected from amino acid residues A^{#} or G^{#}, or
c) Z^{c} is a peptide of the formula:
(SEQ ID NO: 2)
-F^{#}-S^{#}-X^{3c}-W^{#}-G^{#}
wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue.

5. The insecticidal compound according to claim 1, wherein Y is absent or is a peptide comprising from 1 to 14 amino acids.

6. The insecticidal compound according to claim 1, wherein R¹ is selected from:
hydrogen;
acyl optionally substituted with a sugar moiety;
fatty acyl;
heteroaryl;
—NHC₆₋₁₆Aryl;
a sugar moiety;
biotin; or
(poly)ethyleneglycol of the formula *—(OCH₂CH₂)ₙ—Rₚ, where * denotes the point of attachment to L¹, Y¹, Y or Z, n is an integer from 1 to 16, and Rₚ is selected from —NH₂, —OH or —Ome;
optionally wherein R¹ is selected from:
hydrogen;
acyl optionally substituted with a sugar moiety, wherein the acyl group is selected from formyl, acetyl (Ac), propanoyl, butanoyl;
fatty acyl selected from palmitoyl, butyryl, cerotoyl, decanoyl, docosenoyl, dodecanoyl, eleostearoyl, heptanoyl, hexanoyl, icosanoyl, icosenoyl, lignoceroyl, linoleoyl, lipoyl, myristoleoyl, nonanoyl, octadecanoyl, ocatanoyl, palmitoleoyl, stearoyl, undecanoyl, and valeryl;
indolyl;
—NHC₆₋₁₆Aryl;
a monosaccharide or disaccharide moiety;
biotin;
(poly)ethyleneglycol of the formula *—(OCH₂CH₂)ₙ—Rₚ, where * denotes the point of attachment to L¹, Y¹, Y or Z, n is an integer from 4 to 12, and Rₚ is selected from —NH₂, —OH or —Ome;
further optionally wherein R¹ is hydrogen or acetyl.

7. The insecticidal compound according to claim 1, wherein L¹ is absent or is:
—(C═O)C₄₋₁₂-alkylene-NH—, where * denotes the point of attachment to Y¹, Y or Z;
—(C═O)—C₁₋₄alkylene-(C═O)—;
optionally wherein L¹ is absent or is: 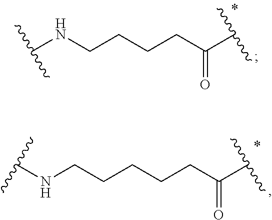 [SMILES: *NCCCCC(*)=O ; *NCCCCCC(*)=O]
where * denotes the point of attachment to Y, Y or Z; or 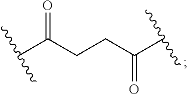 [SMILES: *C(=O)CCC(*)=O]
further optionally wherein L¹ is absent.

8. The insecticidal compound according to claim 1, wherein the Y—Z moiety comprises at least one modified amino acid or a non-natural amino acid analogue.

9. The insecticidal compound according to claim 1, wherein the modified amino acid or non-natural amino acid analogue in peptides Y and Z is selected from:
i. an N-methylated amino acid;
ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
iii. an α-alkylated (e.g. α-methylated) amino acid
iv. a D-amino acid;
v. a beta-amino acid;
vi. a peptoid amino acid analogue;
vii. a sugar modified amino acid; or
viii. a biotin modified amino acid.

10. The insecticidal compound according to claim 1, wherein the modified amino acid or non-natural amino acid analogue in peptides Y and Z is selected from:
i. an N-methylated amino acid;
ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
iii. an α-methylated amino acid;
iv. a D-amino acid; or
v. a beta amino acid.

11. The insecticidal compound according to claim 1, wherein the modified amino acid or non-natural amino acid analogue in peptides Y and Z is selected from:
i. an N-methylated amino acid;
ii. a non-proteinogenic amino acid, e.g. hydroxyproline (Hyp: L-hydroxyproline or (2S,4R)-4-Hydroxyproline), Octahydroindole-2-carboxylic acid (Oic), sarcosine (Sar), norleucine (Nle), α-aminoisobutyric acid (Aib) or thiazolidine-4-carboxylic acid (Thz);
iii. an α-methylated amino acid;
iv. a D-amino acid.

12. The insecticidal compound according to claim 1, wherein the modified or non-natural amino acid is selected from:
i. an N-methylated amino acid;
ii. α-aminoisobutyric acid (Aib);
iii. thiazolidine-4-carboxylic acid (Thz);
iv. an α-methylated amino acid
v. a D-amino acid.

13. The insecticidal compound according to claim 1, wherein R² is R² is NH₂, NR^{2a}H, NR^{2a}R^{2b}, or OR^{2a}; wherein each of R^{2a} and R^{2b} if present is independently C₁₋₆-alkyl (e.g. methyl, ethyl, propyl, butyl, pentyl or hexyl);
optionally wherein R² is NH₂, NR^{2a}H or NR^{2a}R^{2b}, wherein each of R^{2a} and R^{2b} if present is independently C₁₋₆-alkyl (e.g. methyl, ethyl, propyl, butyl, pentyl or hexyl).

14. The insecticidal compound according to claim 1, wherein R² is NH₂.

15. An insecticidal compound having the formula (Ia) below: 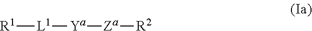
wherein:
R¹, L¹, and R² are as defined in claim 1;
Y^{a} is a group Y as defined in claim 1, optionally wherein
Y^{a} is absent or is a peptide comprising from 1 to 12 amino acids;
Z^{a} is a peptide of the formula:
(SEQ ID NO: 1)
-F^{#}-H^{#}-S^{#}-W^{#}-G^{#};
wherein “^{#}” indicates that the residues “F”, “H”, “S”, “W” and “G” are independently the unmodified amino acid, a modified amino acid or a non-natural amino acid analogue.

16. The insecticidal compound according to claim 15, wherein at least one of the residues in the motif Y^{a}—Z^{a} is a modified amino acid or non-natural amino acid analogue; optionally with the proviso that if the S^{#} residue is an α-aminoisobutyric acid group ([Aib]), then the motif Y^{a}—Z^{a} further comprises at least one D-amino acid.

17. The insecticidal compound according to claim 15, wherein the “S^{#}” residue in the Z motif is a modified amino acid or non-natural amino acid analogue.

18. The insecticidal compound according to claim 15, wherein Y is absent or is a peptide of the formula: 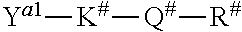
wherein Y^{a1} is absent or a peptide comprising from 1 to 9 amino acids;
i. wherein “^{#}” indicates that the residue is either a naturally occurring amino acid, a modified amino acid or a non-natural amino acid analogue as selected from an N-methylated amino acid, α-aminoisobutyric acid (Aib), thiazolidine-4-carboxylic acid (Thz), an α-methylated amino acid, or a D-amino acid;
optionally wherein Y—Z is a peptide of the formula:
(SEQ ID NO: 13)
Y^{a1}-K^{#}-Q^{#}-R^{#}-F-H^{#}-S^{#}-W-G;
or
(SEQ ID NO: 138)
F-H^{#}-S^{#}-W-G
wherein Y^{a1} is absent or a peptide comprising from 1 to 9 amino acids;
“^{#}” indicates that the residues “K”, “Q”, “R”, “H” and “S” are independently an unmodified amino acid, a modified amino acid or non-natural amino acid analogue;
wherein at least one of the residues “K”, “Q”, “R”, “H” and “S” is a modified amino acid or non-natural amino acid analogue selected from an N-methylated amino acid, α-aminoisobutyric acid (Aib), thiazolidine-4-carboxylic acid (Thz), an α-methylated amino acid, or a D-amino acid.

19. The insecticidal compound according to claim 1, wherein Y—Z is a peptide of the formula:
(SEQ ID NO: 139)
Yₐ₁-K-Q-R_{#}-F-H_{#}-S_{#}-W-G;
or
(SEQ ID NO: 138)
F-H_{#}-S_{#}-W-G
wherein Y^{a1} is absent or a peptide comprising from 1 to 9 amino acids;
wherein “^{#}” indicates that the residues “R”, “H” and “S” are independently an unmodified amino acid, a modified amino acid or non-natural amino acid analogue;
wherein at least one of the residues “R”, “H” and “S” is a modified amino acid or non-natural amino acid analogue selected from an N-methylated amino acid, α-aminoisobutyric acid (Aib), thiazolidine-4-carboxylic acid (Thz), an α-methylated amino acid, or a D-amino acid;
optionally wherein Y^{a}—Z is a peptide of the formula:
(SEQ ID NO: 15)
Y^{a1}-K-Q-R^{#}-F-H-S^{#}-W-G;
or
(SEQ ID NO: 7)
F-H-S^{#}-W-G.
Wherein Y^{a1} is absent or a peptide comprising from 1 to 9 amino acids;
wherein “^{#}” indicates that the residues “R”, and “S” are independently an unmodified amino acid, a modified amino acid or non-natural amino acid analogue;
wherein at least one of the residues “R”, if present, and “S” is a modified amino acid or non-natural amino acid analogue selected from an N-methylated amino acid, α-aminoisobutyric acid (Aib), thiazolidine-4-carboxylic acid (Thz), an α-methylated amino acid, or a D-amino acid.

20. The insecticidal compound according to claim 15, wherein the “S^{#}” residue in the Y^{a} motif is N-methyl serine ([n-me-S]) or D-serine ([s]).

21. The insecticidal compound according to claim 1, wherein the Z^{a} moiety has the formula:
(SEQ ID NO: 40)
FSSWG
(SEQ ID NO: 3)
FH[Aib]WG
(SEQ ID NO: 4)
FHsWG;
or
(SEQ ID NO: 5)
FH[n-me-S]WG;
optionally the Z^{a} moiety has the formula:
(SEQ ID NO: 4)
FHSWG;
or
(SEQ ID NO: 5)
FH[n-me-S]WG.

22. An insecticidal compound selected from:
(SEQ ID NO: 59)
Hy-DPKYKFSSWG-[NH₂];
(SEQ ID NO: 60)
Hy-AKFSSWG-[NH₂];
(SEQ ID NO: 61)
Hy-ARFSSWA-[NH₂];
(SEQ ID NO: 62)
Hy-SKFNSWA-[NH₂];
(SEQ ID NO: 63)
Hy-AKFSSWA-[NH₂];
(SEQ ID NO: 64)
Hy-ARFNSWA-[NH₂];
(SEQ ID NO: 65)
Hy-VPFNSWA-[NH₂];
(SEQ ID NO: 66)
Hy-PIFSSWG-[NH₂];
(SEQ ID NO: 67)
Hy-GPDFYAWG-[NH₂];
(SEQ ID NO: 18)
Ac-KQrFH[Aib]WG-[NH₂];
(SEQ ID NO: 19)
Ac-NSVVLGKKQrFH[Aib]WG-[NH₂];
(SEQ ID NO: 20)
Ac-FH[Aib]WG-[NH₂];
(SEQ ID NO: 21)
Ac-KQrFHsWG-[NH₂];
(SEQ ID NO: 22)
Ac-FHsWG-[NH₂];
(SEQ ID NO: 23)
NSVVLGKKQ[n-me-R]FH[n-me-S]WG-[NH₂];
(SEQ ID NO: 68)
Ac-SKFN-Aib-WA-[NH₂];
(SEQ ID NO: 69)
Ac-SK-[n-me-F]-N-(α-Me) Ser-WA-[NH₂];
(SEQ ID NO: 70)
Ac-PA-[n-me-F]-SsWG-[NH₂];
(SEQ ID NO: 71)
Hy-RQKTVFSSWG-[NH₂];
(SEQ ID NO: 72)
Hy-PAFSSWG-[NH₂];
(SEQ ID NO: 73)
Hy-ASDKHGRPKQTFSSWG-[NH₂];
(SEQ ID NO: 107)
Hy-VRFSPWG-[NH₂];
(SEQ ID NO: 108)
Hy-NFSPWG-[NH₂];
(SEQ ID NO: 109)
Hy-KVKFSAWG-[NH₂];
(SEQ ID NO: 110)
Hy-SFSPWG-[NH₂];
(SEQ ID NO: 111)
Hy-FSPWG-[NH₂];
(SEQ ID NO: 112)
Hy-YFSPWG-[NH₂];
(SEQ ID NO: 113)
Hy-KVKFSVWG-[NH₂];
(SEQ ID NO: 114)
Hy-KVKFSTWG-[NH₂];
(SEQ ID NO: 74)
Ac-PA-[n-me-F]-S-βa-WG-[NH₂];
(SEQ ID NO: 75)
Ac-PA-[n-me-F]-S-(α-Me)S-WG-[NH₂];
(SEQ ID NO: 116)
Ava-FS-BA-WG-[NH₂];
(SEQ ID NO: 117)
Ava-FS-Thz-WG-[NH₂];
(SEQ ID NO: 92)
Hy-GPAFSSWG-[NH₂];
(SEQ ID NO: 93)
Hy-AAAFNSWG-[NH₂];
(SEQ ID NO: 94)
Hy-NAAFSSWG-[nh2];
(SEQ ID NO: 95)
Hy-NAAFNSWG-[NH₂];
(SEQ ID NO: 96)
Hy-VPAFNSWG-[NH₂];
(SEQ ID NO: 97)
Hy-TNTAFSSWG-[NH₂];
(SEQ ID NO: 98)
Hy-PRFYSWG-[NH₂];
(SEQ ID NO: 99)
Hy-GADFYAWG-[NH₂];
(SEQ ID NO: 76)
Hy-DPAFNSWG-[NH₂];
(SEQ ID NO: 77)
Hy-SSAFNSWG-[NH₂];
(SEQ ID NO: 78)
Hy-RAFTSTGSSRSPAFSSWG-[NH₂];
(SEQ ID NO: 79)
Hy-PASFSSWG-[NH₂];
(SEQ ID NO: 80)
Hy-DAGFSSWG-[NH₂];
(SEQ ID NO: 81)
Hy-SSGFSSWG-[NH₂];
(SEQ ID NO: 82)
Hy-DPVFKSWG-[NH₂];
(SEQ ID NO: 83)
Hy-DAAFSSWG-[NH₂];
(SEQ ID NO: 84)
Hy-GSGFSSWG-[NH₂];
(SEQ ID NO: 85)
Hy-DDDSDVSESGSKFSSWG-[NH₂];
(SEQ ID NO: 86)
Hy-SFSSWG-[NH₂];
(SEQ ID NO: 87)
Hy-GFSSWG-[NH₂];
(SEQ ID NO: 88)
Hy-LDRSEGRVSKALFSSWG-[NH₂];
(SEQ ID NO: 89)
Hy-WGQSSVGAVFSSWG-[NH₂];
(SEQ ID NO: 90)
Hy-GAEFYSWG-[NH₂];
(SEQ ID NO: 91)
Hy-FSSWG-[NH₂];
or
(SEQ ID NO: 100)
[Ac]-SK[n-me-F]N[βAla]WA-[NH₂];
or a salt or solvate thereof.

23. A composition, e.g. an insect control composition or plant protection composition, comprising a compound according to claim 1 in admixture with one or more solvents, carriers, diluents, adjuvants, preservatives, dispersants, emulsifying agents, or synergists;
optionally wherein the composition is an aqueous composition; and/or the composition further comprises one or more of a kinin peptide, an AKH peptide, a DH31 peptide, a DH44 peptide, a pyrokinin peptide or a CAPA peptide (e.g. a CAPA-1, CAPA-2 or CAPA-3 analogue)

24. Use, as an insect control agent, of a compound according to claim 1; optionally wherein said use is against insects of the order diptera, hemiptera or lepidoptera;
optionally wherein said use is as an insecticide against:
a) diptera insects, wherein Z is a group Z^{a} and Z^{a} is a peptide of the formula: —F^{#}—H^{#}—S^{#}—W^{#}-G^{#} (SEQ ID NO: 1);
b) hemiptera insects (e.g. Halyomorpha halys, Aphis fabae, Acyrthosiphon pisum, Amrasca biguttula, Myzus persicae or Rhopalosiphum padi insects), wherein Z is a group Z^{b} and Z^{b} is a peptide of the formula: —F^{#}—X^{2b}—X^{3b}—W^{#}—X^{5b}
wherein:
X^{2b} is selected from amino acid residues S^{#}, Y^{#}, K^{#}, N^{#} or H^{#};
X^{3b} is selected from amino acid residues S^{#}, A^{#}, P^{#}, V^{#} or T^{#} (optionally S^{#} or A^{#});
X^{5b} is selected from amino acid residues A^{#} or G^{#};
c) lepidoptera insects (e.g. Plutella xylostella or Spodoptera frugiperda), wherein Z is a group Z^{c} and Z^{c} is a peptide of the formula:
(SEQ ID NO: 2)
-F^{#}-S^{#}-X^{3c}-W^{#}-G^{#}
wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue.

25. A method of increasing insect mortality, the method comprising contacting an insect or insect population with a compound according to claim 1; wherein:
a) the insect population is a diptera insect population, optionally wherein Z is a group Z^{a} and Z^{a} is a peptide of the formula: —F^{#}—H^{#}—S^{#}—W^{#}-G^{#} (SEQ ID NO: 1);
c) b) the insect population is a hemiptera insect population (e.g. Halyomorpha halys, Aphis fabae, Acyrthosiphon pisum, Amrasca biguttula, Myzus persicae or Rhopalosiphum padi), optionally wherein Z is a group Z^{b} and Z^{b} is a peptide of the formula: —F^{#}—X^{2b}—X^{3b}—W^{#}—X^{5b}
wherein:
X^{2b} is selected from amino acid residues S^{#}, Y^{#}, K^{#}, N^{#} or H^{#};
X^{3b} is selected from amino acid residues S^{#}, A^{#}, P^{#}, V^{#} or T^{#} (optionally S^{#} or A^{#});
X^{5b} is selected from amino acid residues A^{#} or G^{#};
c) the insect population is a lepidoptera insect population (e.g. Plutella xylostella or Spodoptera frugiperda), optionally wherein Z is a group Z^{c} and Z^{c} is a peptide of the formula:
(SEQ ID NO: 2)
-F^{#}-S^{#}-X^{3c}-W^{#}-G^{#}
wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue.

26. Use of a compound according to claim 1; as a plant protection agent, for protecting a plant against:
a) diptera insects, optionally wherein Z is a group Z^{a} and Z^{a} is a peptide of the formula: —F^{#}—H^{#}—S^{#}—W^{#}-G^{#} (SEQ ID NO: 1);
b) hemiptera insects (e.g. Halyomorpha halys, Aphis fabae, Acyrthosiphon pisum, Amrasca biguttula, Myzus persicae or Rhopalosiphum padi), optionally wherein Z is a group Z^{b} and Z^{b} is a peptide of the formula: —F^{#}—X^{2b}—X^{3b}—W^{#}—X^{5b}
wherein:
X^{2b} is selected from amino acid residues S^{#}, Y^{#}, K^{#}, N^{#} or H^{#};
X^{3b} is selected from amino acid residues S^{#}, A^{#}, P^{#}, V^{#} or T^{#} (optionally S^{#} or A^{#});
X^{5b} is selected from amino acid residues A^{#} or G^{#};
c) lepidoptera insects (e.g, Plutella xylostella or Spodoptera frugiperda), optionally wherein Z is a group Z^{c} and Z^{c} is a peptide of the formula:
(SEQ ID NO: 2)
-F^{#}-S^{#}-X^{3c}-W^{#}-G^{#}
wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue.

27. A method of inhibiting infestation of a plant by insects, the method comprising contacting the plant with a compound according to claim 1; wherein
a) the insects are diptera insects, optionally wherein Z is a group Z^{a} and Z^{a} is a peptide of the formula: —F^{#}—H^{#}—S^{#}—W^{#}-G^{#} (SEQ ID NO: 1);
b) the insects are hemiptera insects (e.g. Halyomorpha halys, Aphis fabae, Acyrthosiphon pisum, Amrasca biguttula, Myzus persicae or Rhopalosiphum padi), optionally wherein Z is a group Z^{b} and Z^{b} is a peptide of the formula: —F^{#}—X^{2b}—X^{3b}—W^{#}—X^{5b}
wherein:
X^{2b} is selected from amino acid residues S^{#}, Y^{#}, K^{#}, N^{#} or H^{#};
X^{3b} is selected from amino acid residues S^{#}, A^{#}, P^{#}, V^{#} or T^{#} (optionally S^{#} or A^{#});
X^{5b} is selected from amino acid residues A^{#} or G^{#};
c) the insects are lepidoptera insects (e.g, Plutella xylostella or Spodoptera frugiperda), optionally wherein Z is a group Z^{c} and Z^{c} is a peptide of the formula:
(SEQ ID NO: 2)
-F^{#}-S^{#}-X^{3c}-W^{#}-G^{#}
wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue;
optionally wherein the compound is applied to the plant while the plant is free or substantially free of insects.

28. A method of reducing insect infestation of a plant, or of reducing insect load on a plant, the method comprising contacting the plant with a compound according to claim 1; wherein:
a) the insects are diptera insects, optionally wherein Z is a group Z^{a} and Z^{a} is a peptide of the formula: —F^{#}—H^{#}—S^{#}—W^{#}-G^{#} (SEQ ID NO: 1);
b) the insects are hemiptera insects (e.g. Halyomorpha halys, Aphis fabae, Acyrthosiphon pisum, Amrasca biguttula, Myzus persicae or Rhopalosiphum padi), optionally wherein Z is a group Z^{b} and Z^{b} is a peptide of the formula: —F^{#}—X^{2b}—X^{3b}—W^{#}—X^{5b}
wherein:
X^{2b} is selected from amino acid residues S^{#}, Y^{#}, K^{#}, N^{#} or H^{#};
X^{3b} is selected from amino acid residues S^{#}, A^{#}, P^{#}, V^{#} or T^{#} (optionally S^{#} or A^{#}):
X^{5b} is selected from amino acid residues A^{#} or G^{#};
c) the insects are lepidoptera insects (e.g, Plutella xylostella or Spodoptera frugiperda), optionally wherein Z is a group Z^{c} and Z^{c} is a peptide of the formula:
(SEQ ID NO: 2)
-F^{#}-S^{#}-X^{3c}-W^{#}-G^{#}
wherein X^{3c} is selected from amino acid residues A^{#}, P^{#}, V^{#} or T^{#};
and wherein “^{#}” indicates that the listed residue is independently the unmodified amino acid, a modified amino acid or a non-natural amino acid analogue.


SEQUENCE LISTING (139 sequences)

SEQ ID NO: 1 - protein, 5 aa, synthetic construct
  VARIANT 1..5: note = unmodified amino acid, modified amino acid, or a non-natural amino acid analogue
FHSWG

SEQ ID NO: 2 - protein, 5 aa, synthetic construct
  VARIANT 1..2: note = unmodified amino acid, modified amino acid, or non-natural amino acid analogue
  VARIANT 4..5: note = unmodified amino acid, modified amino acid, or non-natural amino acid analogue
  VARIANT 3: note = X may be A, P, V, T wherein each of these is independently the unmodified amino acid, a modified amino acid, or a non-natural amino acid analogue
FSXWG

SEQ ID NO: 3 - protein, 5 aa, synthetic construct
  SITE 3: note = Aib
FHXWG

SEQ ID NO: 4 - protein, 5 aa, synthetic construct
  SITE 3: note = D-serine
FHSWG

SEQ ID NO: 5 - protein, 5 aa, synthetic construct
  SITE 3: note = N-methyl serine
FHSWG

SEQ ID NO: 6 - protein, 5 aa, synthetic construct
  source 1..5: note = ZaI
  SITE 2..3: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue, a sugar modified amin acid, or a biotin modified amino acid
  VARIANT 2..3: note = at least one of H or S is X, an N-methylated amino acid, a D-amino acid, an a-alkylated (e.g. a-methylated) amino acid, or a beta-amino acid
FHSWG

SEQ ID NO: 7 - protein, 5 aa, synthetic construct
  SITE 3: note = X, N-methyl-L-serine, N-methyl-D-serine, D-serine, alpha-alkyl-L-serine (e.g. alpha-methyl-L-serine), alpha-alkyl-D-serine (e.g. alpha-methyl-D-serine), beta-L-serine, beta-D-serine
  SITE 3: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue of serine, a sugar modified serine, or a biotin modified serine
FHSWG

SEQ ID NO: 8 - protein, 5 aa, synthetic construct
  VARIANT 3: note = Aib, N-methyl serine, or D-serine
FHSWG

SEQ ID NO: 9 - protein, 5 aa, synthetic construct
  VARIANT 3: note = N-methyl serine or D-serine
FHSWG

SEQ ID NO: 10 - protein, 5 aa, synthetic construct
FHSWG

SEQ ID NO: 11 - protein, 10 aa, synthetic construct
  SITE 10: note = D-arginine
NSVVLGKKQR

SEQ ID NO: 12 - protein, 10 aa, synthetic construct
  SITE 10: note = n-methyl arginine
NSVVLGKKQR

SEQ ID NO: 13 - protein, 17 aa, synthetic construct
  VARIANT 10..12: note = unmodified amino acid, modified amino acid, or non-natural amino acid analogue
  VARIANT 14..15: note = unmodified amino acid, modified amino acid, or non-natural amino acid analogue
  VARIANT 1..9: note = any number (0 to 9) of these residues may be deleted
  VARIANT 10..15: note = at least one of residues K, Q, R, H and S is X, an N-methylated amino acid, a D-amino acid, an a-alkylated (e.g. a-methylated) amino acid, or a beta-amino acid
  SITE 10..15: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue, a sugar modified amino acid, or a biotin modified amino acid
XXXXXXXXXKQRFHSWG

SEQ ID NO: 14 - protein, 17 aa, synthetic construct
  VARIANT 1..9: note = any number (0 to 9) of these residues may be deleted
  VARIANT 12: note = X, N-methyl-L-arginine, N-methyl-D-arginine, D-arginine, alpha-alkyl-L-arginine (e.g. alpha-methyl-L-arginine), alpha-alkyl-D-arginine (e.g. alpha-methyl-D-arginine), beta-L-arginine, beta-D-arginine
  VARIANT 12..15: note = at least one of residues R, H and S is X, an N-methylated amino acid, a D-amino acid, an a-alkylated (e.g. a-methylated) amino acid, or a beta-amino acid
  SITE 12: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue of arginine, a sugar modified arginine, or a biotin modified arginine
  VARIANT 14: note = X, N-methyl-L-histidine, N-methyl-D-histidine, D-histidine, alpha-alkyl-L-histidine (e.g. alpha-methyl-L-histidine), alpha-alkyl-D-histidine (e.g. alpha-methyl-D-histidine), beta-L-histidine, beta-D-histidine
  SITE 14..15: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue of histidine, a sugar modified histidine, or a biotin modified histidine
  VARIANT 15: note = X, N-methyl-L-serine, N-methyl-D-serine, D-serine, alpha-alkyl-L-serine (e.g. alpha-methyl-L-serine), alpha-alkyl-D-serine (e.g. alpha-methyl-D-serine), beta-L-serine, beta-D-serine
XXXXXXXXXKQRFHSWG

SEQ ID NO: 15 - protein, 17 aa, synthetic construct
  VARIANT 1..9: note = any number (0 to 9) of these residues may be deleted
  VARIANT 12: note = X, N-methyl-L-arginine, N-methyl-D-arginine, D-arginine, alpha-alkyl-L-arginine (e.g. alpha-methyl-L-arginine), alpha-alkyl-D-arginine (e.g. alpha-methyl-D-arginine), beta-L-arginine, beta-D-arginine
  VARIANT 15: note = X, N-methyl-L-serine, N-methyl-D-serine, D-serine, alpha-alkyl-L-serine (e.g. alpha-methyl-L-serine), alpha-alkyl-D-serine (e.g. alpha-methyl-D-serine), beta-L-serine, beta-D-serine
  VARIANT 12..15: note = at least one of residues R and S is X, an N-methylated amino acid, a D-amino acid, an a-alkylated (e.g. a-methylated) amino acid, or a beta-amino acid
  SITE 12: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue of arginine, a sugar modified arginine, or a biotin modified arginine
  SITE 15: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue of serine, a sugar modified serine, or a biotin modified serine
XXXXXXXXXKQRFHSWG

SEQ ID NO: 16 - protein, 17 aa, synthetic construct
  VARIANT 1..9: note = any number (0 to 9) of these residues may be deleted
  SITE 15: note = X, N-methyl-L-serine, N-methyl-D-serine, D-serine, alpha-alkyl-L-serine (e.g. alpha-methyl-L-serine), alpha-alkyl-D-serine (e.g. alpha-methyl-D-serine), beta-L-serine, beta-D-serine
  VARIANT 15: note = unmodified amino acid, modified amino acid, or non-natural amino acid analogue
  SITE 12: note = X, N-methyl-L-arginine, N-methyl-D-arginine, D-arginine, alpha-alkyl-L-arginine (e.g. alpha-methyl-L-arginine), alpha-alkyl-D-arginine (e.g. alpha-methyl-D-arginine), beta-L-arginine, beta-D-arginine
  SITE 12..15: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue, a sugar modified amino acid, or a biotin modified amino acid
XXXXXXXXXKQRFHSWG

SEQ ID NO: 17 - protein, 17 aa, synthetic construct
  VARIANT 1..9: note = any number (0 to 9) of these residues may be deleted
  VARIANT 12: note = N-methyl arginine or D-arginine
  VARIANT 15: note = N-methyl serine or D-serine
XXXXXXXXXKQRFHSWG

SEQ ID NO: 18 - protein, 8 aa, unidentified
  source 1..8: note = SB-P-032
  SITE 3: note = D-arginine
  SITE 6: note = Aib
  SITE 1: note = acetyl-L-lysine
  SITE 8: note = glycinamide
KQRFHXWG

SEQ ID NO: 19 - protein, 15 aa, synthetic construct
  source 1..15: note = SB-P-033
  SITE 1: note = acetyl-L-asparagine
  SITE 10: note = D-arginine
  SITE 13: note = Aib
  SITE 15: note = glycinamide
NSVVLGKKQRFHXWG

SEQ ID NO: 20 - protein, 5 aa, synthetic construct
  source 1..5: note = SB-P-034
  SITE 1: note = acetyl-L-phenylalanine
  SITE 3: note = Aib
  SITE 5: note = glycinamide
FHXWG

SEQ ID NO: 21 - protein, 8 aa, synthetic construct
  source 1..8: note = SB-P-035
  SITE 1: note = acetyl-L-lysine
  SITE 3: note = D-arginine
  SITE 6: note = D-serine
  SITE 8: note = glycinamide
KQRFHSWG

SEQ ID NO: 22 - protein, 5 aa, synthetic construct
  source 1..5: note = SB-P-036
  SITE 1: note = acetyl-L-phenylalanine
  SITE 3: note = D-serine
  SITE 5: note = glycinamide
FHSWG

SEQ ID NO: 23 - protein, 15 aa, synthetic construct
  source 1..15: note = SB-P-052
  SITE 10: note = N-methyl arginine
  SITE 13: note = N-methyl serine
  SITE 15: note = glycinamide
NSVVLGKKQRFHSWG

SEQ ID NO: 24 - protein, 5 aa, synthetic construct
  source 1..5: note = ZbI
  VARIANT 1..5: note = unmodified amino acid, modified amino acid, or non-natural amino acid analogue
FSSWG

SEQ ID NO: 25 - protein, 5 aa, synthetic construct
  source 1..5: note = ZbII
  VARIANT 1..5: note = unmodified amino acid, modified amino acid, or non-natural amino acid analogue
FSSWA

SEQ ID NO: 26 - protein, 5 aa, synthetic construct
  source 1..5: note = ZbIII
  VARIANT 1..5: note = unmodified amino acid, modified amino acid, or non-natural amino acid analogue
FNSWA

SEQ ID NO: 27 - protein, 5 aa, synthetic construct
  source 1..5: note = ZbIV
  VARIANT 1..5: note = unmodified amino acid, modified amino acid, or non-natural amino acid analogue
FYSWG

SEQ ID NO: 28 - protein, 5 aa, synthetic construct
  source 1..5: note = ZbV
  VARIANT 1..5: note = unmodified amino acid, modified amino acid, or non-natural amino acid analogue
FNAWA

SEQ ID NO: 29 - protein, 5 aa, unidentified
  source 1..5: note = ZbVI
  VARIANT 1..5: note = unmodified amino acid, modified amino acid, or non-natural amino acid analogue
FSAWG

SEQ ID NO: 30 - protein, 5 aa, synthetic construct
  source 1..5: note = ZbVII
  VARIANT 1..5: note = unmodified amino acid, modified amino acid, or non-natural amino acid analogue
FNSWG

SEQ ID NO: 31 - protein, 5 aa, synthetic construct
  source 1..5: note = ZbVIII
  VARIANT 1..5: note = unmodified amino acid, modified amino acid, or non-natural amino acid analogue
FKSWG

SEQ ID NO: 32 - protein, 5 aa, synthetic construct
  source 1..5: note = ZbI-I
  VARIANT 1: note = unmodified amino acid, modified amino acid, or non-natural amino acid analogue
  VARIANT 3: note = unmodified amino acid, modified amino acid, or non-natural amino acid analogue
FSSWG

SEQ ID NO: 33 - protein, 5 aa, synthetic construct
  source 1..5: note = ZbII-I
  VARIANT 1: note = unmodified amino acid, modified amino acid, or non-natural amino acid analogue
  VARIANT 3: note = unmodified amino acid, modified amino acid, or non-natural amino acid analogue
FSSWA

SEQ ID NO: 34 - protein, 5 aa, synthetic construct
  source 1..5: note = ZbIII-I
  VARIANT 1: note = unmodified amino acid, modified amino acid, or non-natural amino acid analogue
  VARIANT 3: note = unmodified amino acid, modified amino acid, or non-natural amino acid analogue
FNSWA

SEQ ID NO: 35 - protein, 5 aa, synthetic construct
  source 1..5: note = ZbIV-I
  VARIANT 1: note = unmodified amino acid, modified amino acid, or non-natural amino acid analogue
  VARIANT 3: note = unmodified amino acid, modified amino acid, or non-natural amino acid analogue
FYSWG

SEQ ID NO: 36 - protein, 5 aa, synthetic construct
  source 1..5: note = ZbV-I
  VARIANT 1: note = unmodified amino acid, modified amino acid, or non-natural amino acid analogue
  VARIANT 3: note = unmodified amino acid, modified amino acid, or non-natural amino acid analogue
FNAWA

SEQ ID NO: 37 - protein, 5 aa, synthetic construct
  source 1..5: note = ZbVI-I
  VARIANT 1: note = unmodified amino acid, modified amino acid, or non-natural amino acid analogue
  VARIANT 3: note = unmodified amino acid, modified amino acid, or non-natural amino acid analogue
FSAWG

SEQ ID NO: 38 - protein, 5 aa, synthetic construct
  source 1..5: note = ZbVII-I
  VARIANT 1: note = unmodified amino acid, modified amino acid, or non-natural amino acid analogue
  VARIANT 3: note = unmodified amino acid, modified amino acid, or non-natural amino acid analogue
FNSWG

SEQ ID NO: 39 - protein, 5 aa, synthetic construct
  source 1..5: note = ZbVIII-I
  VARIANT 1: note = unmodified amino acid, modified amino acid, or non-natural amino acid analogue
  VARIANT 3: note = unmodified amino acid, modified amino acid, or non-natural amino acid analogue
FKSWG

SEQ ID NO: 40 - protein, 5 aa, synthetic construct
FSSWG

SEQ ID NO: 41 - protein, 5 aa, synthetic construct
FSSWA

SEQ ID NO: 42 - protein, 5 aa, synthetic construct
FNSWA

SEQ ID NO: 43 - protein, 5 aa, synthetic construct
FYAWG

SEQ ID NO: 44 - protein, 5 aa, synthetic construct
  SITE 3: note = Aib
FNXWA

SEQ ID NO: 45 - protein, 5 aa, synthetic construct
  SITE 3: note = bAla
FNAWA

SEQ ID NO: 46 - protein, 5 aa, synthetic construct
  SITE 1: note = N-methyl phenylalanine
  SITE 3: note = D-serine
FSSWG

SEQ ID NO: 47 - protein, 5 aa, synthetic construct
  SITE 1: note = N-methyl phenylalanine
  SITE 3: note = bAla
FSSWG

SEQ ID NO: 48 - protein, 5 aa, synthetic construct
  SITE 1: note = N-methyl phenylalanine
  SITE 3: note = alpha-methyl-L-serine
FSSWG

SEQ ID NO: 49 - protein, 5 aa, synthetic construct
FNSWG

SEQ ID NO: 50 - protein, 5 aa, synthetic construct
FKSWG

SEQ ID NO: 51 - protein, 5 aa, synthetic construct
DPKYK

SEQ ID NO: 52 - protein, 5 aa, synthetic construct
RQKTV

SEQ ID NO: 53 - protein, 11 aa, synthetic construct
ASDKHGRPKQT

SEQ ID NO: 54 - protein, 4 aa, synthetic construct
TNTA

SEQ ID NO: 55 - protein, 13 aa, synthetic construct
RAFTSTGSSRSPA

SEQ ID NO: 56 - protein, 12 aa, synthetic construct
DDDSDVSESGSK

SEQ ID NO: 57 - protein, 12 aa, synthetic construct
LDRSEGRVSKAL

SEQ ID NO: 58 - protein, 9 aa, synthetic construct
WGQSSVGAV

SEQ ID NO: 59 - protein, 10 aa, synthetic construct
  source 1..10: note = SB-P-003
  SITE 10: note = glycinamide
DPKYKFSSWG

SEQ ID NO: 60 - protein, 7 aa, synthetic construct
  source 1..7: note = SB-P-004
  SITE 7: note = glycinamide
AKFSSWG

SEQ ID NO: 61 - protein, 7 aa, synthetic construct
  source 1..7: note = SB-P-018
  SITE 7: note = alaninamide
ARFSSWA

SEQ ID NO: 62 - protein, 7 aa, synthetic construct
  source 1..7: note = SB-P-019
  SITE 7: note = alaninamide
SKFNSWA

SEQ ID NO: 63 - protein, 7 aa, synthetic construct
  source 1..7: note = SB-P-020
  SITE 7: note = alaninamide
AKFSSWA

SEQ ID NO: 64 - protein, 7 aa, synthetic construct
  source 1..7: note = SB-P-021
  SITE 7: note = alaninamide
ARFNSWA

SEQ ID NO: 65 - protein, 7 aa, synthetic construct
  source 1..7: note = SB-P-022
  SITE 7: note = alaninamide
VPFNSWA

SEQ ID NO: 66 - protein, 7 aa, synthetic construct
  source 1..7: note = SB-P-023
  SITE 7: note = glycinamide
PIFSSWG

SEQ ID NO: 67 - protein, 8 aa, synthetic construct
  source 1..8: note = SB-P-024
  SITE 8: note = glycinamide
GPDFYAWG

SEQ ID NO: 68 - protein, 7 aa, synthetic construct
  source 1..7: note = SB-P-054
  SITE 1: note = acetyl-L-serine
  SITE 2: note = D-lysine
  SITE 5: note = Aib
  SITE 7: note = alaninamide
SKFNXWA

SEQ ID NO: 69 - protein, 9 aa, unidentified
  source 1..9: note = SB-P-056
  SITE 3: note = N-methyl phenylalanine
  SITE 5: note = alpha-methyl-L-serine
  SITE 6: note = D-glutamic acid
  SITE 7: note = D-arginine
  SITE 9: note = alaninamide
SKFNSERWA

SEQ ID NO: 70 - protein, 7 aa, synthetic construct
  source 1..7: note = SB-P-060
  SITE 1: note = acetyl-proline
  SITE 3: note = N-methyl phenylalanine
  SITE 5: note = D-serine
  SITE 7: note = glycinamide
PAFSSWG

SEQ ID NO: 71 - protein, 10 aa, synthetic construct
  source 1..10: note = SB-P-065
  SITE 10: note = glycinamide
RQKTVFSSWG

SEQ ID NO: 72 - protein, 7 aa, synthetic construct
  source 1..7: note = SB-P-066
  SITE 7: note = glycinamide
PAFSSWG

SEQ ID NO: 73 - protein, 16 aa, synthetic construct
  source 1..16: note = SB-P-067
  SITE 16: note = glycinamide
ASDKHGRPKQTFSSWG

SEQ ID NO: 74 - protein, 7 aa, synthetic construct
  source 1..7: note = SB-P-100
  SITE 1: note = acetyl-proline
  SITE 3: note = N-methyl phenylalanine
  SITE 5: note = bAla
  SITE 7: note = glycinamide
PAFSAWG

SEQ ID NO: 75 - protein, 7 aa, synthetic construct
  source 1..7: note = SB-P-101
  SITE 1: note = acetyl-proline
  SITE 3: note = N-methyl phenylalanine
  SITE 5: note = alpha-methyl-L-serine
  SITE 7: note = glycinamide
PAFSSWG

SEQ ID NO: 76 - protein, 8 aa, synthetic construct
  source 1..8: note = SB-P-143
  SITE 8: note = glycinamide
DPAFNSWG

SEQ ID NO: 77 - protein, 8 aa, synthetic construct
  source 1..8: note = SB-P-144
  SITE 8: note = glycinamide
SSAFNSWG

SEQ ID NO: 78 - protein, 18 aa, synthetic construct
  source 1..18: note = SB-P-145
  SITE 18: note = glycinamide
RAFTSTGSSRSPAFSSWG

SEQ ID NO: 79 - protein, 8 aa, synthetic construct
  source 1..8: note = SB-P-146
  SITE 8: note = glycinamide
PASFSSWG

SEQ ID NO: 80 - protein, 8 aa, synthetic construct
  source 1..8: note = SB-P-147
  SITE 8: note = glycinamide
DAGFSSWG

SEQ ID NO: 81 - protein, 8 aa, synthetic construct
  source 1..8: note = SB-P-148
  SITE 8: note = glycinamide
SSGFSSWG

SEQ ID NO: 82 - protein, 8 aa, synthetic construct
  source 1..8: note = SB-P-149
  SITE 8: note = glycinamide
DPVFKSWG

SEQ ID NO: 83 - protein, 8 aa, synthetic construct
  source 1..8: note = SB-P-150
  SITE 8: note = glycinamide
DAAFSSWG

SEQ ID NO: 84 - protein, 8 aa, synthetic construct
  source 1..8: note = SB-P-151
  SITE 8: note = glycinamide
GSGFSSWG

SEQ ID NO: 85 - protein, 17 aa, synthetic construct
  source 1..17: note = SB-P-154
  SITE 17: note = glycinamide
DDDSDVSESGSKFSSWG

SEQ ID NO: 86 - protein, 6 aa, synthetic construct
  source 1..6: note = SB-P-155
  SITE 6: note = glycinamide
SFSSWG

SEQ ID NO: 87 - protein, 6 aa, synthetic construct
  source 1..6: note = SB-P-156
  SITE 6: note = glycinamide
GFSSWG

SEQ ID NO: 88 - protein, 17 aa, synthetic construct
  source 1..17: note = SB-P-157
  SITE 17: note = glycinamide
LDRSEGRVSKALFSSWG

SEQ ID NO: 89 - protein, 14 aa, synthetic construct
  source 1..14: note = SB-P-158
  SITE 14: note = glycinamide
WGQSSVGAVFSSWG

SEQ ID NO: 90 - protein, 8 aa, synthetic construct
  source 1..8: note = SB-P-159
  SITE 8: note = glycinamide
GAEFYSWG

SEQ ID NO: 91 - protein, 5 aa, synthetic construct
  source 1..5: note = SB-P-169
  SITE 5: note = glycinamide
FSSWG

SEQ ID NO: 92 - protein, 8 aa, synthetic construct
  SITE 8: note = glycinamide
GPAFSSWG

SEQ ID NO: 93 - protein, 8 aa, synthetic construct
  SITE 8: note = glycinamide
AAAFNSWG

SEQ ID NO: 94 - protein, 8 aa, synthetic construct
  SITE 8: note = glycinamide
NAAFSSWG

SEQ ID NO: 95 - protein, 8 aa, synthetic construct
  SITE 8: note = glycinamide
NAAFNSWG

SEQ ID NO: 96 - protein, 8 aa, synthetic construct
  SITE 8: note = glycinamide
VPAFNSWG

SEQ ID NO: 97 - protein, 9 aa, synthetic construct
  SITE 9: note = glycinamide
TNTAFSSWG

SEQ ID NO: 98 - protein, 7 aa, synthetic construct
  SITE 7: note = glycinamide
PRFYSWG

SEQ ID NO: 99 - protein, 8 aa, synthetic construct
  SITE 8: note = glycinamide
GADFYAWG

SEQ ID NO: 100 - protein, 7 aa, synthetic construct
  SITE 1: note = acetyl-serine
  SITE 3: note = N-methyl phenylalanine
  SITE 5: note = bAla
  SITE 7: note = alaninamide
SKFNAWA

SEQ ID NO: 101 - protein, 5 aa, synthetic construct
FSPWG

SEQ ID NO: 102 - protein, 5 aa, synthetic construct
FSAWG

SEQ ID NO: 103 - protein, 5 aa, synthetic construct
FSVWG

SEQ ID NO: 104 - protein, 5 aa, synthetic construct
FSTWG

SEQ ID NO: 105 - protein, 5 aa, synthetic construct
  SITE 3: note = bAla
FSAWG

SEQ ID NO: 106 - protein, 5 aa, synthetic construct
  SITE 3: note = thiazolidine-4-carboxylic acid
FSXWG

SEQ ID NO: 107 - protein, 7 aa, synthetic construct
  source 1..7: note = SB-P-068
  SITE 7: note = glycinamide
VRFSPWG

SEQ ID NO: 108 - protein, 6 aa, synthetic construct
  source 1..6: note = SB-P-069
  SITE 6: note = glycinamide
NFSPWG

SEQ ID NO: 109 - protein, 8 aa, synthetic construct
  source 1..8: note = SB-P-070
  SITE 8: note = glycinamide
KVKFSAWG

SEQ ID NO: 110 - protein, 6 aa, synthetic construct
  source 1..6: note = SB-P-071
  SITE 6: note = glycinamide
SFSPWG

SEQ ID NO: 111 - protein, 5 aa, synthetic construct
  source 1..5: note = SB-P-072
  SITE 5: note = glycinamide
FSPWG

SEQ ID NO: 112 - protein, 6 aa, synthetic construct
  source 1..6: note = SB-P-073
  SITE 6: note = glycinamide
YFSPWG

SEQ ID NO: 113 - protein, 8 aa, synthetic construct
  source 1..8: note = SB-P-074
  SITE 8: note = glycinamide
KVKFSVWG

SEQ ID NO: 114 - protein, 8 aa, synthetic construct
  source 1..8: note = SB-P-075
  SITE 8: note = glycinamide
KVKFSTWG

SEQ ID NO: 115 - protein, 7 aa, synthetic construct
  source 1..7: note = SB-P-076
  SITE 7: note = glycinamide
VRFSPWG

SEQ ID NO: 116 - protein, 6 aa, synthetic construct
  source 1..6: note = SB-P-102
  SITE 1: note = 5-aminovaleric acid
  SITE 4: note = bAla
  SITE 6: note = glycinamide
XFSAWG

SEQ ID NO: 117 - protein, 6 aa, synthetic construct
  source 1..6: note = SB-P-103
  SITE 1: note = 5-aminovaleric acid
  SITE 4: note = thiazolidine-4-carboxylic acid
  SITE 6: note = glycinamide
XFSXWG

SEQ ID NO: 118 - protein, 5 aa, synthetic construct
  source 1..5: note = ZcI
  VARIANT 3: note = X may be A, P, V, T wherein each of these is independently the unmodified amino acid, a modified amino acid, or a non-natural amino acid analogue
FSXWG

SEQ ID NO: 119 - protein, 5 aa, synthetic construct
  source 1..5: note = ZcI-I
  VARIANT 3: note = unmodified amino acid, modified amino acid, or non-natural amino acid analogue
FSAWG

SEQ ID NO: 120 - protein, 5 aa, synthetic construct
  source 1..5: note = ZcI-II
  VARIANT 3: note = unmodified amino acid, modified amino acid, or non-natural amino acid analogue
FSPWG

SEQ ID NO: 121 - protein, 5 aa, synthetic construct
  source 1..5: note = ZcI-III
  VARIANT 3: note = unmodified amino acid, modified amino acid, or non-natural amino acid analogue
FSVWG

SEQ ID NO: 122 - protein, 5 aa, synthetic construct
  source 1..5: note = ZcI-IV
  VARIANT 3: note = unmodified amino acid, modified amino acid, or non-natural amino acid analogue
FSTWG

SEQ ID NO: 123 - protein, 12 aa, synthetic construct
  source 1..12: note = SB-P-46
  SITE 12: note = leucinamide
SPPYSPPFSPRL

SEQ ID NO: 124 - protein, 11 aa, synthetic construct
  source 1..11: note = SB-P-47
  SITE 1: note = pyroglutamyl-alanine
  SITE 11: note = leucinamide
AIMARPQVPRL

SEQ ID NO: 125 - protein, 19 aa, synthetic construct
  source 1..19: note = SB-P-48
  SITE 19: note = leucinamide
EQNVQSNGEPAYRVRTPRL

SEQ ID NO: 126 - protein, 8 aa, synthetic construct
  source 1..8: note = SB-P-49
  SITE 8: note = leucinamide
SVPFKPRL

SEQ ID NO: 127 - protein, 19 aa, synthetic construct
  source 1..19: note = SB-P-51
  SITE 19: note = leucinamide
LRQLQSNGEPAYRVRTPRL

SEQ ID NO: 128 - protein, 16 aa, synthetic construct
  source 1..16: note = SB-P-80
  SITE 16: note = leucinamide
NADEDQQQSVDFTPRL

SEQ ID NO: 129 - protein, 10 aa, synthetic construct
  source 1..10: note = SB-P-81
  SITE 10: note = leucinamide
GGSMTFSPRL

SEQ ID NO: 130 - protein, 9 aa, synthetic construct
  source 1..9: note = SB-P-86
  SITE 1: note = pyroglutamyl-leucine
  SITE 9: note = glycinamide
LTFTSSWGG

SEQ ID NO: 131 - protein, 8 aa, synthetic construct
  source 1..8: note = SB-P-39
  SITE 1: note = palmitoyl-glutamine
  SITE 8: note = tryptophanamide
QLTFSPDW

SEQ ID NO: 132 - protein, 8 aa, synthetic construct
  source 1..8: note = SB-P-41
  SITE 8: note = tryptophanamide
QLTFSPDW

SEQ ID NO: 133 - protein, 7 aa, synthetic construct
  source 1..7: note = SB-P-42
  SITE 1: note = pyroglutamyl-leucine
  SITE 7: note = tryptophanamide
LTFSPDW

SEQ ID NO: 134 - protein, 14 aa, synthetic construct
  source 1..14: note = SB-P-015
  SITE 7: note = N-methyl alanine
  SITE 14: note = leucinamide
SDSKNTALWFGPRL

SEQ ID NO: 135 - protein, 10 aa, synthetic construct
  source 1..10: note = 2315
  SITE 4: note = beta-homoleucine (3-amino-5-methylcaproic acid)
  SITE 10: note = leucinamide
ASGLVAFPRV

SEQ ID NO: 136 - protein, 7 aa, synthetic construct
  source 1..7: note = AH59
  SITE 1: note = palmitoyl-leucine
  SITE 7: note = valinamide
LVAFPRV

SEQ ID NO: 137 - protein, 8 aa, synthetic construct
  source 1..8: note = AH270
  SITE 1: note = 6-aminohexanoic acid (also known as 6-aminocaproic acid or epsilon-aminocaproic acid)
  SITE 4: note = N-methyl alanine
  SITE 8: note = N-methyl valinamide
XLVAFPRV

SEQ ID NO: 138 - protein, 5 aa, synthetic construct
  VARIANT 2..3: note = unmodified amino acid, modified amino acid, or non-natural amino acid analogue
  VARIANT 2..3: note = at least one of H or S is X, an N-methylated amino acid, a D-amino acid, an a-alkylated (e.g. a-methylated) amino acid, or a beta-amino acid
  SITE 2..3: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue, a sugar modified amin acid, or a biotin modified amino acid
FHSWG

SEQ ID NO: 139 - protein, 17 aa, synthetic construct
  VARIANT 1..9: note = any number (0 to 9) of these residues may be deleted
  VARIANT 12: note = unmodified amino acid, modified amino acid, or non-natural amino acid analogue
  VARIANT 14..15: note = unmodified amino acid, modified amino acid, or non-natural amino acid analogue
  VARIANT 12..15: note = at least one of residues R, H and S is X, an N-methylated amino acid, a D-amino acid, an a-alkylated (e.g. a-methylated) amino acid, or a beta-amino acid
  VARIANT 12..15: note = X may be a non-proteinogenic amino acid e.g. hydroxyproline (L-hydroxyproline or (2S,4R)-4-Hydroxyproline), octahydroindole-2-carboxylic acid, sarcosine, Nle, Aib, or thiazolidine-4-carboxylic acid; a peptoid amino acid analogue, a sugar modified amino acid, or a biotin modified amino acid
XXXXXXXXXKQRFHSWG